(19) United States
(12) Patent Application Publication
(10) Pub. No.: US 2013/0156737 A1
(43) Pub. Date: Jun. 20, 2013

(54) Secretion System and Methods for its Use

(71) Applicant: University of Washington through its Center for, Seattle, WA (US)

(72) Inventors: Joseph Mougous, Seattle, WA (US); Alistair Brian Russell, Seattle, WA (US); Michele LeRoux, Seattle, WA (US)

(73) Assignee: University of Washington through its Center for Commercialization, Seattle, WA (US)

(21) Appl. No.: 13/632,417

(22) Filed: Oct. 1, 2012

Related U.S. Application Data
(63) Continuation of application No. 13/180,975, filed on Jul. 12, 2011, now abandoned.

(51) Int. Cl. A61K 38/16; C07K 14/21

(52) U.S. Cl. CPC A61K 38/164; C07K 14/21
USPC 424/93.2; 530/350; 514/1.1; 435/252.34; 435/252.3; 435/252.33; 536/23.4; 435/198; 514/2.4; 435/320.1

(57) ABSTRACT

The present invention provides reagents and methods for inhibiting bacterial infection and abnormal cell growth, as well as for selection cloning of nucleic acid inserts.

### CROSS REFERENCE

This application is a continuation of U.S. application Ser. No. 13/180,975 filed Jul. 12, 2011 which claims priority to U.S. Provisional Patent Application Ser. Nos. 61/497,808 filed Jun. 16, 2011 and 61/489,039 filed May 23, 2011, and U.S. patent application Ser. No. 12/970,390 filed Dec. 16, 2010, which claims priority to U.S. Provisional Patent Application Ser. No. 61/286,899 filed Dec. 16, 2009, all of which are incorporated by reference herein in its entirety.

### STATEMENT OF U.S. GOVERNMENT INTEREST

This work was funded in part by NIH Grant Nos. AI080609 and AI057141. The U.S. government has certain rights in the invention.

### BACKGROUND

Bacterial infection and abnormal cell growth are causative factors in a variety of disease states and environmental contamination. Thus, developing new reagents and methods to inhibit bacterial infection and abnormal cell growth are of substantial importance.

### SUMMARY OF THE INVENTION

In a first aspect, the present invention provides substantially purified type VI secretion exported (Tse) proteins, selected from the group consisting of Tse1, Tse2, and Tse3, and type VI secretion immunity proteins selected from the group consisting of Tsi1, Tsi2, and Tsi3.

In a second aspect, the present invention provides substantially purified nucleic acids encoding Tse and/or Tsi protein-conjugates of any embodiment of the invention.

In a third aspect, the present invention provides a vector comprising the substantially purified nucleic acid of any embodiment of the second aspect of the invention, wherein the substantially purified nucleic acid is operatively linked to a regulatory sequence.

In a fourth aspect, the present invention provides host cells comprising the recombinant expression vector of any embodiment of the third aspect of the invention.

In a fifth aspect, the present invention provides pharmaceutical compositions comprising the substantially purified protein of any embodiment of the invention; and

(b) a pharmaceutically acceptable carrier.

In a sixth aspect, the invention provides host cells comprising,

(a) a plurality of genes encoding proteins capable of forming a type 6 secretion system (T6SS); and

(b) a recombinant gene encoding a therapeutic polypeptide that can be secreted by the recombinant T6SS in the recombinant cell, wherein the recombinant gene is operatively linked to a regulatory sequence. In one embodiment, the recombinant gene encodes a fusion polypeptide of (a) a therapeutic polypeptide selected from the group consisting of bactericidal proteins group IIA phospholipase A2, bactericidal/permeability-increasing protein, human peptidoglycan recognition proteins 3 and 4 (PGLYRP3 and PGLYRP4), Tse1, Tse2, Tse3, or other native T6SS substrates, or functional equivalents thereof; and (b) one or both of a VgrG polypeptide and a Hcp polypeptide. Thus, the present invention also provides novel fusion polypeptides comprising (a) a therapeutic polypeptide selected from the group consisting of bactericidal proteins group IIA phospholipase A2, bactericidal/permeability-increasing protein, human peptidoglycan recognition proteins 3 and 4 (PGLYRP3 and PGLYRP4), Tse1, Tse2, Tse3, or other native T6SS substrates, or functional equivalents thereof; and (b) one or both of a VgrG polypeptide and a Hcp polypeptide, and novel genes encoding such polypeptides.

In a seventh aspect, the present invention provides pharmaceutical compositions comprising (a) the recombinant host cells of the sixth aspect of the invention; and (b) a pharmaceutically acceptable carrier.

In an eighth aspect, the present invention provides an anti-bacterial composition comprising the recombinant host cell or polypeptide of any embodiment disclosed herein adhered to a substrate.

In a ninth aspect, the present invention provides methods for inhibiting bacterial growth, comprising contacting bacteria to be inhibited with an amount of the host cells of any embodiment of the invention or the substantially purified polypeptide of any embodiment of the invention effective to inhibit bacterial growth.

In a tenth aspect, the present invention provides methods for inhibiting eukaryotic growth, comprising contacting eukaryotic cells to be inhibited with an amount of the substantially purified Tse conjugates of the first aspect of the invention effective to inhibit eukaryotic cell growth.

In an eleventh aspect, the present invention provides recombinant vectors, comprising a first gene coding for Tse1 and/or Tse3, of functional equivalents thereof, wherein the first gene is operatively linked to a heterologous regulatory sequence.

In a twelfth aspect, the present invention provides recombinant host cells comprising the recombinant vector of any embodiment or combination of embodiments of the eleventh aspect of the invention.

In a thirteenth aspect, the present invention provides methods for selectable cloning, comprising culturing the recombinant host cell of any embodiment of the twelfth aspect of the invention under conditions suitable for expression of Tse1 and/or Tse3 from the recombinant vector if no insert is present, and selecting those cells that grow as comprising recombinant vectors with the insert cloned into the expression vector.

In a fourteenth aspect, the present invention provides methods for producing a cloning vector that lacks an insert, comprising culturing the recombinant host cell of any embodiment of the twelfth aspect of the invention under conditions suitable for vector replication and expression of Tse1 and/or Tse3, wherein the recombinant host cells further express a Tse1 and/or Tse3 antidote, and isolating vector from the host cells.

In a fifteenth aspect, the invention provides methods for improved biomolecule extraction from bacterial cells, comprising contacting the bacterial cells with an amount effective of Tse1 to lyse the bacterial cells during the extraction process.

### BRIEF DESCRIPTION OF THE FIGURES

FIG. 1. Overview and results of an MS-based screen to identify H1-T6SS substrates. (A) Gene organization of P. aeruginosa HSI-I. Genes manipulated in this work are shown in color. (B) Activity of the H1-T6SS can be modulated by deletions of pppA and clpV1. Western blot analysis of Hcp1-V in the cell-associated (Cell) and concentrated supernatant (Sup) protein fractions from P. aeruginosa strains of specified genetic backgrounds. The genetic background for the parental strain is indicated below the blot. An antibody directed against RNA polymerase α (α-RNAP) is used as a loading control in this and subsequent blots. (C) Deletion of pppA causes increased p-Fha1-V levels. p-Fha1-V is observed by Western blot as one or more species with retarded electrophoretic mobility. (D) Spectral count ratio of C1 proteins detected in R1 and R2 of the comparative semi-quantitative secretome analysis of ΔpppA and ΔclpV1. The position of Hcp1 in both replicates is indicated. Proteins within the dashed line have SC ratios of <2-fold and constitute 89% of C1 proteins.

FIG. 2. Two VgrG-family proteins are regulated by retS and secreted in an H1-T6SS-dependent manner. (A) Overview of genetic loci encoding C2 proteins identified in R1 and R2 (green). RetS regulation of each ORF as determined by Goodman et al. is provided (Goodman et al., 2004). Genes not significantly regulated by RetS are filled grey. (B and C) Western blot analysis demonstrating that secretion of VgrG1-V (B) and VgrG4-V (C) is triggered in the ΔpppA background and is H1-T6SS (clpV1)-dependent. All blots are against the VSV-G epitope (α-VSV-G).

FIG. 3. The Tse proteins are tightly regulated H1-T6SS substrates. (A) Tse secretion is under tight negative regulation by pppA and is H1-T6SS-dependent. Western analysis of Tse proteins expressed with C-terminal VSV-G epitope tag fusions from pPSV35 (Rietsch et al., 2005). Unless otherwise noted, all blots in this figure are α-VSV-G. (B) H1-T655-dependent secretion of chromosomally-encoded Tse1-V measured by Western blot analysis. (C) Hcp1 secretion is independent of the tse genes. Western blot analysis of Hcp1 localization in control strains or strains lacking both vgrG1 and vgrG4, or the three tse genes. (D) The tse genes are not required for formation of a critical H1-T6S apparatus complex. Chromosomally-encoded ClpV1-GFP localization in the specified genetic backgrounds measured by fluorescence microscopy. TMA-DPH is a lipophilic dye used to visualize the position of cells. (E) The production and secretion of Tse proteins is dramatically increased in ΔretS. Western blot analysis of Tse levels from strains containing chromosomally-encoded Tse-VSV-G epitope tag fusions prepared in the wild-type or ΔretS backgrounds. Note—under conditions used to observe the high levels of Tse secretion in ΔretS, secretion cannot be visualized in ΔpppA as was demonstrated in (B).

FIG. 4. The Tse2 and Tsi2 proteins are a toxin-immunity module. (A) Tse2 is toxic to P. aeruginosa in the absence of Tsi2. Growth of the indicated P. aeruginosa strains containing either the vector control (−) or vector containing tse2 (+) under non-inducing (−IPTG) or inducing (+IPTG) conditions. (B) Tse2 and Tsi2 physically associate. Western blot analysis of samples before (Pre) and after (Post) α-VSV-G immunoprecipitation from the indicated strain containing a plasmid expressing tsi2 (control) or tsi2-V. The glycogen synthase kinase (GSK) tag was used for detection of Tse2 (Garcia et al., 2006).

FIG. 5. Heterologously expressed Tse2 is toxic to prokaryotic and eukaryotic cells. (A) Tse2 is toxic to S. cerevisiae. Growth of S. cerevisiae cells containing a vector control or a vector expressing the indicated tse under non-inducing (Glucose) or inducing (Galactose) conditions. (B) Tsi2 blocks the toxicity of Tse2 in S. cerevisiae. Growth of S. cerevisiae harboring plasmids with the indicated gene(s), or empty plasmid(s), under non-inducing or inducing conditions. (C, D and E) Transfected Tse2 has a pronounced effect on mammalian cells. Flow cytometry (C) and fluorescence microscopy (D) analysis of GFP reporter co-transfection experiments with plasmids expressing the tse genes or tsi2. The percentage of rounded cells following the indicated transfections was determined (E) (n>500). Control (ctrl) experiments contained only the reporter plasmid. Bar graphs represent the average number from at least three independent experiments (±SEM). (F and G) Expression of tse2 inhibits the growth of E. coli (F) and B. thailandensis (G). E. coli (F) and B. thailandensis (G) were transformed with expression plasmids regulated by inducible expression with IPTG (F) or rhamnose (G), respectively, containing no insert, tse2, or both the tse2 and tsi2 loci. Growth on solid medium was imaged after one (F) or two (G) days of incubation.

FIG. 6. Immunity to Tse2 provides a growth advantage against P. aeruginosa strains secreting the toxin by the H1-T6SS. (A) Tse2 secreted by the H1-T6SS of P. aeruginosa does not promote cytotoxicity in HeLa cells. LDH release by HeLa cells following infection with the indicated P. aeruginosa strains or E. coli. P. aeruginosa strain PA14 and E. coli were included as highly cytotoxic and non-cytotoxic controls, respectively. Bars represent the mean of five independent experiments ±SEM. (B and C) Results of in vitro growth competition experiments in liquid medium (B) or on a solid support (B and C) between P. aeruginosa strains of the indicated genotypes. The parental strain is ΔretS. The ΔclpV1 and Δtsi2-dependent effects were complemented as indicated by +clpV1 and +tsi2, respectively (see methods). Bars represent the mean donor:recipient CFU ratio from three independent experiments (±SEM).

FIG. 7. The Burkholderia T6SSs cluster with eukaryotic and prokaryotic-targeting systems in a T6S phylogeny. (A) Overview of the B. thai T6SS gene clusters. Burkholderia T6SS-3 is absent from B.thai. Genes were identified according to the nomenclature proposed by Shalom and colleagues [28]: tss, type six secretion conserved genes; tag, type six secretion-associated genes that are variably present in T6SSs. Genes are colored according to function and conservation (dark grey, tss genes; light grey, tag genes; color, experimentally characterized tss or tag genes; white, genes so far not linked to T6S. Brackets demarcate genes that were deleted in order to generate B. thai strains ΔT6SS-1, -2, -4-5 and -6 and their assorted combinations. (B) Neighbor-Joining tree based on 334 T6S-associated VipA orthologs. The locations of VipA proteins from T6SSs discussed in the text are indicated. Each line represents one or more orthologous T6SSs from a single species. Lines are colored based on bacterial taxonomy of the corresponding organism. Indicated bootstrap values correspond to 100 replicates.

FIG. 8. Of the five B. thai T6SSs, only T6SS-5 is required for virulence in a murine acute melioidosis model. C57BL/6 wild-type mice were infected by the aerosol-route with 10⁵ cfu/lung of B. thai strains and monitored for survival for 10-14 days post infection (p.i.). Survival of mice after exposure to B. thai wild-type, (A) strains harboring gene deletions in individual T6SS gene clusters (n=5), (B) a strain bearing an in-frame tssK-5 deletion (ΔtssK-5) or its complemented derivative (ΔtssK-5-comp; n=7 and 8, respectively), (C) or a strain with inactivating mutations in four T6SSs (ΔT6SS-1,2,4,6; n=8).

FIG. 9. B. thai ΔtssK-5 shows a replication defect in the lung of wild-type mice but is highly virulent in MyD88^{-/-} mice. Mice were exposed to 10⁵ cfu/lung aerosolized B. thai wild-type or ΔtssK-5 bacteria and c.f.u. were monitored in the (A) lung after 4, 24, and 48 h (n=6 per time point), and in the (B) liver and spleen after 24 and 48 h (n=6 per time point). (C) C57BL/6 wild-type (n=6) and MyD88^{-/-} mice (n=7) were infected with ΔtssK-5 strain and survival was monitored for 14 d. Error bars in (A) and (B) are ±SD.

FIG. 10. T6S plays a role in the fitness of B. thai in growth competition assays with other bacteria. (A) In vitro growth of B. thai wild-type and a strain bearing gene deletions in all five T6SSs (ΔT6S). (B) B. thai wild-type and ΔT6S swimming motility in semi-solid LB agar (scale bar=1.0 cm). (C) Fluorescence images of growth competition assays between GFP-labeled B. thai wild-type and ΔT6S strains against the indicated unlabeled competitor species. Competition assay outcomes could be divided into T6S-independent (AR, Agrobacterium rhizogenes; ATu, A. tumefaciens; AV, A. vitis; PD, Paracoccus denitrificans; RS, Rhodobacter sphaeroides; ATe, Acidovorax temperans; BT, B. thailandensis; BV, B. vietnamiensis; AC, Acinetobacter calcoaceticus; AH, Aeromonas hydrophile; ECa, Erwinia carotovora; FN, Francisella novicida; PA, Pseudomonas aeruginosa; SM, Serratia marcescens; VC, Vibrio cholerae; VV, V. vulnificus; XC, Xanthomonas campestris; XN, Xenorhabdus nematophilus; YP, Yersinia pestis LCR⁻; BC, Bacillus cereus; BS, B. subtilis; ML, Micrococcus luteus; SA, Staphylococcus aureus; SP, Streptococcus pyogenes), those with modest T6S-effects (BA, B. ambifaria; ECo, E. coli; KP, Klebsiella pneumoniae; ST, Salmonella typhimurium) and those in which B. thai proliferation was strongly T6S-dependent (dashed boxes—PP, P. putida E0044; PF, P. fluorescens ATCC27663; SP, S. proteamaculans 568). The latter are referred to as TDCs (type VI secretion-dependent competitors). This latter group of organisms are referred to as the T6S-dependent competitors (TDCs).

FIG. 11. T6SS-1 is involved in cell contact-dependent interbacterial interactions. (A) Growth competition assays between the indicated GFP-labeled B. thai strains and the TDCs. Standard light photographs and fluorescent images of the competition assays are shown. (B) Fluorescence images of GFP-labeled B. thai wild-type and ΔT6SS-1 grown in the presence of the TDCs with (no contact, NC) or without (contact, C) an intervening filter. (C) Fluorescence images of growth competition assays between GFP-labeled B. that ΔclpV-1 or complemented ΔclpV-1 with the TDCs. (D) Quantification of c.f.u before (initial) and after (final) growth competition assays between the indicated organisms. The c.f.u. ratio of the B. thai strain versus competitor bacteria is plotted. Error bars represent ±SD.

FIG. 12. T6SS-1 is required for resistance against P. putida-induced growth inhibition. (A-C) B. thai and P. putida growth following inoculation of competitive cultures (A,B) or mono-cultures (C) onto LB 3% w/v agar. (D,E) B. thai and P. putida growth following inoculation of competitive cultures into LB broth. (F) Quantification of dead cells 7.5 h after initiating competition between P. putida and the indicated B. thai strain on LB 3% w/v agar (n>7,000). Error bars are ±SD.

FIG. 13. T6SS-1 is required for B. thai to persist in mixed biofilm with P. putida. Fluorescence confocal microscopy images of B. thai (green) and P. putida (cyan) biofilm formation in flow chambers. (A) Representative images of monotypic B. thai biofilms of the indicated strains immediately following seeding (Day 0) and after four days of maturation. (B) Representative images of mixed biofilms seeded with a 1:1 mixture of P. putida with the indicated B. thai strains.

FIG. 14. Heterologous expression of periplasmic-targeted Tse1 and Tse3 in E. coli. Experiments were carried out in BL21 pLysS E. coli. Proteins Tse1 and Tse3 were expressed downstream of a T7 promoter with a C-terminal His tag either cytoplasmically in pET29b+, or periplasmically using the pelB signal sequence in pET22b+. Cells were initially grown at 37° C. shaking overnight in LB supplemented with 25 μg/ml chloramphenicol and 100 μg/ml carbenicillin (pET22b+) or 50 μg/mlkanamycin (pET29b+). Overnight cultures were then diluted to an OD of approximately 0.05 in no salt-LB supplemented with 100 μg/ml carbenicillin (pET22b+) or 50 μg/mlkanamycin (pET29b+) and grown in 200 μl volumes in a 96 well plate. Tse expression was induced with 0.1 mM IPTG in logarithmic phase (point of induction indicated by arrows in the figure.

FIG. 15. Tse1 and Tse3 are lytic proteins belonging to amidase and muramidase enzyme families.

a. Genomic organization of tse1 and tse3 and their homology with characterized amidase and muramidase enzymes, respectively. Highly conserved (boxed) and catalytic (starred) residues of the respective enzyme families are indicated. SWISS-PROT entry names for the proteins shown are: Tse1 (Q912Q1_PSEAE), Spr (SPR_ECOLI), P60 (P60_LISIN), Tse3 (Q9HYC5_PSEAE), GEWL (LYG_ANSAN), Slt70 (SLT_ECOLI).
b,d. Partial HPLC chromatograms of sodium borohydride-reduced soluble E. coli peptidoglycan products resulting from (b) digestion with Tse1 and subsequent cleavage with cellosyl or (d) digestion with Tse3 alone. Peak assignments were made based on MS; predicted structures are shown schematically with hexagons and circles corresponding to sugars and amino acid residues, respectively. Reduced sugar moieties are shown with grey fill.
c. Simplified representation of Gram-negative peptidoglycan showing cleavage sites of Tse1 and Tse3 based on data summarized in b and d.
e. Growth in liquid media of E. coli producing the indicated peri-Tse proteins. Periplasmic localization was achieved by fusion to the PelB leader sequence³⁵. Cultures were induced at the indicated time (arrow). Error bars ±s.d. (n=3).
f. Representative micrographs of strains shown in e acquired prior to complete lysis. The lipophilic dye TMA-DPH is used to highlight the cellular membranes. All images were acquired at the same magnification. Scale bar=2 μm.

FIG. 16. Tse1 and Tse3 are not required for Tse2 export or transfer to recipient cells via the T6S apparatus.

a. Western blot analysis of supernatant (Sup) and cell-associated (Cell) fractions of the indicated P. aeruginosa strains. The parental background for all experiments represented in this figure is PAO1 ΔretS, a strain in which the H1-T6SS is activated constitutivelyl^{13,36}.
b. Growth competition assays between the indicated donor and recipient strains under T6S-conducive conditions. Experiments were initiated with equal colony forming units (c.f.u.) of donor and recipient bacteria as denoted by the dashed line. The ΔclpV1 strain is a T6S-deficient control. Asterisks indicate significant differences in competition outcome between recipient strains against the same donor strain. **P<0.01. Error bars ±s.d. (n=3).

FIG. 17. Tsi1 and Tsi3 provide immunity to cognate toxins.

a. Western blot analysis of hexahistidine-tagged Tse proteins (—His₆) in total and bead-associated fractions of an α-VSV-G (vesicular stomatitis virus glycoprotein) immunoprecipitation of VSV-G epitope fused Tsi proteins (—V) from E. coli.
b. Growth of E. coli harboring a vector expressing the indicated tse gene (top panels) or vectors expressing the indicated tse and tsi genes (bottom panels). Numbers at top indicate 10-fold serial dilutions.
c. Fluorescence micrographs showing colony growth of the indicated strains. The parental background for this experiment was PAO1 ΔretS attTn7::gfp. Growth of the Δtsi strains was rescued by the addition of 1.0% w/v NaCl to the underlying medium.
d. Replication rates of the indicated P. aeruginosa strains in liquid medium of low osmolarity formulated as in c. The parental strain used in this experiment was PAO1 ΔretS. Error bars ±s.d. (n=3).

FIG. 18. Tse1 and Tse3 delivered to the periplasm provide a fitness advantage to donor cells.

a. Western blot analyses of cytoplasmic (Cyto) and periplasmic (Peri) fractions of P. aeruginosa strains producing Tsi1-V, Tsi3-V or Tsi3-SS-V. Equivalent ratios of the Cyto and Peri samples were loaded in each panel. RNA polymerase (RNAP) and β-lactamase (β-lac) enzymes were used as cytoplasmic and periplasmic fractionation controls, respectively. The presence of Tsi3—a predicted outer membrane lipoprotein—in the periplasmic fraction is consistent with previous studies utilizing this method of fractionation³⁷.

b. Growth competition assays between the indicated donor and recipient strains under T6S-conducive conditions. Experiments were initiated with equal c.f.u. of donor and recipient bacteria as denoted by the dashed line. The parental strain used in this experiment was PAO1 ΔretS. All donor strains were modified at the attB site with lacZ. Asterisks indicate outcomes significantly different than parental versus Δtse3 Δtsi3 (top bar). Error bars ±s.d. (n=4). **P<0.01.
c. Lysis of EDTA-permeabilized or intact P. aeruginosa cells with equal quantities of Tse1, Tse1*, or Lysozyme (Ly). Lysis was normalized to a buffer control. Error bars ±s.d. (n=3).
d. Competitive growth of P. aeruginosa against P. putida on solid (open circles) or in liquid (filled circles) medium. Competition outcome was defined as the c.f.u. ratio (P. aeruginosa/P. putida) divided by the initial ratio. The dotted line represents the boundary between competitions that increase in P. aeruginosa relative to P. putida (above the line) and those that increase in P. putida relative to P. aeruginosa (below the line). The parental strain used in this experiment was P. aeruginosa PAO1. Asterisks above competitions denote those where the outcome (P. aeruginosa/P. putida) was significantly less than the parental (P<0.05). Horizontal bars denote the average value for each dataset (n=5).

FIG. 19. Proposed mechanism of T6S-dependent delivery of effector proteins. The schematic depicts the junction between competing bacteria, with a donor cell delivering the Tse effector proteins through the T6S apparatus (grey tube) to recipient cell periplasm. Effector and immunity proteins are shown as circles and rounded rectangles, respectively. Bonds in the peptidoglycan that are predicted targets of the effector proteins are highlighted. Cytoplasm (C), inner membrane (IM), periplasm (P), and outer membrane (OM) of both bacteria are shown.

### DETAILED DESCRIPTION OF THE INVENTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2^{nd} Ed. (R.I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

As used herein, the singular forms “a”, “an” and “the” include plural referents unless the context clearly dictates otherwise. “And” as used herein is interchangeably used with “or” unless expressly stated otherwise.

All embodiments of any aspect of the invention can be used in combination, unless the context clearly dictates otherwise.

In a first aspect, the present invention provides substantially purified type VI secretion exported (Tse) proteins, selected from the group consisting of Tse1, Tse2, and Tse3. As shown in the examples that follow, Tse2 is a Pseudomonas aeruginosa protein that is toxic to a broad spectrum of prokaryotic and eukaryotic cells, and thus can be used, for example, as therapeutics to destroy deleterious cells of interest, while Tse1 and Tse3 have potent antibacterial activity, and thus can be used in any suitable antibacterial application. The inventors have also shown that Tse1 possesses bacterial lytic activity, and thus it can also be used, for example, in methods for improved biomolecule extraction from bacterial cells, as discussed below.

This aspect also provides substantially purified type VI secretion immunity (Tsi) proteins, selected from the group consisting of Tsi1, Tsi2, and Tsi3. As shown in the examples that follow, Tsi1, Tsi2, and Tsi3 are Pseudomonas aeruginosa proteins that confer immunity to the Tse proteins disclosed herein that are toxic to a broad spectrum of prokaryotic and eukaryotic cells (Tse2) or have potent antibacterial activity (Tse1 and Tse3), and thus can be used, for example, in embodiments of the methods disclosed herein. As used herein, “substantially purified” means that the polypeptide has been separated from its in vivo cellular environments. It is further preferred that the isolated polypeptides are also substantially free of gel agents, such as polyacrylamide, agarose, and chromatography reagents.

In one preferred embodiment, the Tse is Tse2 and comprises or consists of the P. aeruginosa amino acid sequence according to SEQ ID NO:2. Closely related Tse2 proteins are present in other P. aeruginosa strains, with variable positions noted in SEQ ID NOS:4. Thus, in another preferred embodiment, Tse2 comprises or consists of an amino acid sequence according to SEQ ID NO:4.

As used herein, “Tse2” includes functional equivalents (truncations, mutants, etc.) thereof, wherein such equivalents maintain cytotoxic activity as described herein. Methods for identifying such functional equivalents are disclosed herein and a variety of such functional equivalents are disclosed. The inventors have discovered that residues 1-6 and 156-158 of Tse2 are not required for toxicity (See Table 1 below). Thus, in another embodiment, the first gene comprises a nucleotide sequence that can encode an amino acid sequence according to SEQ ID NO:5 or SEQ ID NO:6.

The inventors have further identified a series of Tse2 mutant polypeptides that retain toxicity. Specifically, the inventors have shown (see below) that mutations at positions 9, 10, 60, 119, 129, 130, 139, 140, 149, and 150 of SEQ ID NO:2 can be tolerated while retaining toxicity (See Table 2 below). Thus, in another embodiment, the first gene encodes a mutant Tse2 polypeptide that differs from the amino acid sequence of SEQ ID NO:2 by an amino acid substitution at one or more of amino acid residues 9, 10, 60, 119, 129, 130, 139, 140, 149, and 150, and is optionally deleted for one or more of resides 1-6 and one or more of residues 156-158. In another embodiment, the first gene encodes a mutant Tse2 polypeptide that includes one or more amino acid substitutions selected from the group consisting of S9A. L10A, R60A, Q119A, K129A, P129A, Q139A, L139A, R149A, and R150A. In a further preferred embodiment, the first gene comprises a nucleotide sequence that can encode an amino acid sequence according to SEQ ID NO:7 or SEQ ID NO:8.

In another preferred embodiment, the Tse is Tse1 and comprises or consists of the amino acid sequence according to SEQ ID NO:10. In a further preferred embodiment, the Tse is Tse3 and comprises or consists of the amino acid sequence according to SEQ ID NO:12.

As used herein, “Tse1” and “Tse3” includes functional equivalents (truncations, mutants, etc.) thereof, wherein such equivalents maintain antibacterial activity as described herein. Methods for identifying such functional equivalents are disclosed herein and a variety of such functional equivalents are disclosed.

In another embodiment, the substantially purified Tsi1 protein comprises or consists of the amino acid sequence according to SEQ ID NO:54. In a further embodiment, the substantially purified Tsi3 protein comprises or consists of the amino acid sequence according to SEQ ID NO:56. In a further embodiment, the substantially purified Tsi2 protein comprises or consists of the amino acid sequence according to SEQ ID NO:55.

As used herein, “Tsi1,” “Tsi2,” and “Tsi3” includes functional equivalents (truncations, mutants, etc.) thereof, wherein such equivalents maintain immunity activity as described herein. Methods for identifying such functional equivalents are disclosed herein.

In a further preferred embodiment of any embodiment disclosed above, the substantially purified Tse protein or Tsi protein comprises a Tse or Tsi-conjugate. As disclosed below, Tse2 is toxic to cells (prokaryotic and eukaryotic) when expressed intracellularly, while Tse1 and Tse3 have anti-bacterial activity, and thus conjugates that can serve to move the Tse2 proteins into cells, or that serve to allow Tse1 and/or Tse3 to be transported to the periplasmic space are useful, for example, in various methods disclosed herein. In one embodiment, the conjugates comprise Tse2-transduction domain conjugates. As used herein, the term “transduction domain” means one or more amino acid sequence or any other molecule that promote the intracellular delivery of cargo that would otherwise fail to, or only minimally, traverse the cell membrane. These domains can be linked, for example, to other polypeptides to direct movement of the linked polypeptide across cell membranes. Thus, the Tse-transduction fusion proteins can be used to directly administer the Tse toxins to deleterious cells. A wide variety of such transduction domains are known in the art, including but not limited to

(SEQ ID NO: 13)
GRKKRRQRRRPPQ
(SEQ ID NO: 14)
RQIKIWFQNRRMKWKK
(SEQ ID NO: 15)
RRMKWKK
(SEQ ID NO: 16)
RGGRLSYSRRRFSTSTGR
(SEQ ID NO: 17)
RRLSYSRRRF
(SEQ ID NO: 18)
RGGRLAYLRRRWAVLGR
(SEQ ID NO: 19)
RRRRRRRR.
(SEQ ID NO: 20)
YGRKKRRQRRR,
(SEQ ID NO: 21)
ILLPLLLLP,
(SEQ ID NO: 22)
RQLKIWFQNRRMKWKK,
(SEQ ID NO: 23)
RKKRRQRRR,
(SEQ ID NO: 24)
YARAAARQARA,
(SEQ ID NO: 25)
RRQRRTSKLMKR,
(SEQ ID NO: 26)
AAVLLPVLLAAR,
(SEQ ID NO: 27)
RRRRRRRRR,
(SEQ ID NO: 28)
SGWFRRWKK,
(SEQ ID NO: 29)
RQIKIWFQNRRMKWKK,
(SEQ ID NO: 30)
(R)₄₋₉,
(SEQ ID NO: 31)
GRKKRRQRRRPPQ,
(SEQ ID NO: 32)
DAATATRGRSAASRPTERPRAPARSASRPRRPVE,
(SEQ ID NO: 33)
GWTLNSAGYLLGLINLKALAALAKKIL,
(SEQ ID NO: 34)
PLSSIFSRIGDP,
(SEQ ID NO: 35)
AAVALLPAVLLALLAP,
(SEQ ID NO: 36)
AAVLLPVLLAAP,
(SEQ ID NO: 37)
VTVLALGALAGVGVG,
(SEQ ID NO: 38)
GALFLGWLGAAGSTMGAWSQP,
(SEQ ID NO: 39)
GWTLNSAGYLLGLINLKALAALAKKIL,
((SEQ ID NO: 40)
KLALKLALKALKAALKLA,
(SEQ ID NO: 41)
KETWWETWWTEWSQPKKKRKV,
(SEQ ID NO: 42)
KAFAKLAARLYRKAGC,
(SEQ ID NO: 43)
KAFAKLAARLYRAAGC,
(SEQ ID NO: 44)
AAFAKLAARLYRKAGC,
(SEQ ID NO: 45)
KAFAALAARLYRKAGC,
(SEQ ID NO: 46)
KAFAKLAAQLYRKAGC,
(SEQ ID NO: 47)
GGGGYGRKKRRQRRR,
and
(SEQ ID NO: 48)
YGRKKRRQRRR.

In any of these embodiments, the substantially purified Tse protein-transduction domain conjugate preferably comprises the general formula XI-Tse-X2, wherein X1 and X2 independently comprise a transduction domain or are absent, with the proviso that at least one of X1 and X2 are present.

In other embodiments, the Tse protein comprises a conjugate comprising the Tse protein and one or more of the following:

(a) a targeting domain to carry the conjugate across a bacterial outer membrane to a periplasmic space, including but not limited to colicin (or domains thereof) and liposomes;

(b) a phage capsid;

(c) a leader sequence to target intracellular Tse protein to the periplasmic space, including but not limited to PelB, or domains thereof.

Each of these conjugates can be used, for example, to assist movement of the Tse proteins into cells under certain conditions. For example, the targeting domain can be used to move the conjugate across a bacterial outer membrane to a periplasmic space (i.e.: permits extracellular conjugate to into the periplasmic space for antibacterial activity of Tse proteins). The leader sequence can be used to target intracellular Tse to the periplasmic space (i.e.: recombinant gene expressing the Tse operatively linked to a leader sequence). It is well within the level of skill in the art to use liposomes, phage capsids, and other constructs as delivery devices for a Tse protein to target cells, based on the teachings herein.

In another preferred embodiment that can be combined with any of the above embodiments, the Tse or Tsi protein or Tse or Tsi-conjugate further comprises a cell targeting molecule. As used herein, a “cell targeting molecule” is a molecule, such as a polypeptide, that binds to a cell surface receptor, to facilitate cell specific targeting of the Tse protein. Any suitable cell targeting molecule can be used that is appropriate for a given purpose. In various non-limiting embodiments, the cell targeting molecule is selected from the group consisting of a tumor targeting molecule such as transferrin or folate; an amino acid sequence which consists of the amino acids arginine, followed by glycine and aspartate (also known as an RGD motif) for targeting epithelial and endothelial cells; glycoside or lectin-containing molecules to facilitate targeting of lectin expressing tumor cells, macrophages, hepatocytes and parenchymal cells; and a monoclonal antibody, or fragment thereof, which can bind to the chosen target cells, such as cancer cells of a desired target type.

In another embodiment, the Tse1 or Tse3 is a fusion protein that comprises a secretory signal sequence. The term “secretory signal sequence” or “signal sequence” are described, for example in U.S. Pat. Nos. 6,291,212 and 5,547,871, both of which are herein incorporated by reference in their entirety.

In a second aspect, the present invention provides substantially purified nucleic acids encoding the Tse or Tsi protein conjugates of any embodiment of the invention. As used herein, a “nucleic acid” includes DNA, RNA, mRNA, cDNA, and analogs thereof, whether single stranded or double stranded.

As used herein, “substantially purified nucleic acids” are those that have been removed from their normal surrounding nucleic acid sequences. Such substantially purified nucleic acid sequences may comprise additional sequences useful for promoting expression and/or purification of the encoded protein, including but not limited to polyA sequences, modified Kozak sequences, and sequences encoding epitope tags, export signals, and secretory signals, nuclear localization signals, and plasma membrane localization signals.

In a third aspect, the present invention provides a vector comprising the substantially purified nucleic acid of any embodiment of the second aspect of the invention, wherein the substantially purified nucleic acid is operatively linked to a regulatory sequence. Any suitable vectors can be used, including but not limited to plasmid and viral vectors.

Vectors, such as expression vectors and methods for their engineering and isolation are well known in the art (see, e.g., Maniatis et al., supra), or they can be obtained through a commercial vendor, e.g., Invitrogen (Carlsbad, Calif.), Promega (Madison, Wis.), and Statagene (La Jolla, Calif.) and modified as needed. Examples of commercially available expression vectors include pcDNA3 (Invitrogen), Gateway cloning technology (Life Technologies), and pCMV-Script (Stratagene). Vector components, regulatory nucleic acids, etc. are typically available from a commercial source or can be isolated from a natural source (e.g., animal tissue or microorganism) or prepared using a synthetic means such as PCR. The arrangement of the components can be any arrangement practically desired by one of ordinary skill in the art.

In a fourth aspect, the present invention provides host cells comprising the recombinant expression vector of any embodiment of the third aspect of the invention, wherein the host cells can be either prokaryotic or eukaryotic. The cells can be transiently or stably transfected. Such transfection of expression vectors into prokaryotic and eukaryotic cells can be accomplished via any technique known in the art, including but not limited to standard bacterial transformations, calcium phosphate co-precipitation, electroporation, or liposome mediated-, DEAE dextran mediated-, polycationic mediated-, or viral mediated transfection. (See, for example, Molecular Cloning: A Laboratory Manual (Sambrook, et al., 1989, Cold Spring Harbor Laboratory Press; Culture of Animal Cells: A Manual of Basic Technique, 2^{nd} Ed. (R.I. Freshney. 1987. Liss, Inc. New York, N.Y.).

In a fifth aspect, the present invention provides pharmaceutical compositions comprising the substantially purified protein of any embodiment of the invention; and

(b) a pharmaceutically acceptable carrier.

In a preferred embodiment, the substantially purified proteins for use in the pharmaceutical compositions are selected from the group consisting of Tse 1, Tse2, and Tse3. For administration, the proteins are ordinarily combined with one or more adjuvants appropriate for the indicated route of administration. The proteins may be admixed with lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, dextran sulfate, heparin-containing gels, and/or polyvinyl alcohol, and tableted or encapsulated for conventional administration. Alternatively, the proteins may be dissolved in saline, water, polyethylene glycol, propylene glycol, carboxymethyl cellulose colloidal solutions, ethanol, corn oil, peanut oil, cottonseed oil, sesame oil, tragacanth gum, and/or various buffers. Other adjuvants and modes of administration are well known in the pharmaceutical art. The carrier or diluent may include time delay material, such as glyceryl monostearate or glyceryl distearate alone or with a wax, or other materials well known in the art.

The proteins may be made up in a solid form (including granules, powders or suppositories) or in a liquid form (e.g., solutions, suspensions, or emulsions). The proteins may be applied in a variety of solutions. Suitable solutions for use in accordance with the invention are sterile, dissolve sufficient amounts of the polypeptides, and are not harmful for the proposed application.

In a sixth aspect, the invention provides host cells comprising,

(a) a plurality of genes encoding proteins capable of forming a type 6 secretion system (T6SS); and

(b) a recombinant gene encoding a therapeutic polypeptide that can be secreted by the recombinant T6SS in the recombinant cell, wherein the recombinant gene is operatively linked to a regulatory sequence.

As disclosed below, the inventor has discovered that T6SSs can be used to deliver polypeptide therapeutics to other bacteria, and thus can be used for targeting toxins (or other proteins/macromolecules) to bacteria.

The “host cells” can be any host cell capable of expressing T6SS endogenously or by recombinant means, and secreting the therapeutic polypeptide via the T6SS. Type VI secretion systems have been found in most genomes of proteobacteria, including animal, plant, human pathogens, as well as soil, environmental and marine bacteria. In one embodiment, the host cell is a bacterial cell and the T6SS is the endogenous T6SS expressed by that bacteria. In a preferred embodiment, the bacterial cell is a gram negative bacteria, including but not limited to P. fluorescens, Burkholderia thai, P. putida, proteobacteria including but not limited to Escherichia coli, Salmonella, Shigella, and other Enterobacteriaceae, Pseudomonas, Moraxella, Helicobacter, Stenotrophomonas, Bdellovibrio, acetic acid bacteria, Legionella, Wolbachia, cyanobacteria, spirochaetes, green sulfur and green non-sulfur bacteria, Hemophilus influenzae, Klebsiella pneumoniae, Legionella pneumophila, Pseudomonas aeruginosa, Proteus mirabilis, Enterobacter cloacae, Serratia sp (including but not limited to Serratia marcescens), Helicobacter pylori, Salmonella enteritidis (including but not limited to Salmonella typhi and Salmonella typhimurium) Acinetobacter baumannii, Pseudomonas aeruginosa, Burkholderia cepacia complex species (including but not limited to Burkholderia cepacia), Burkholderia seudomallei, Ralstonia picketti, Acinetobacter baumanii, Klebsiella pneuominiae, Proteus mirabilis, Chromobacterium violaceum, Bordetella sp. (parapertussis, bronchiseptics, petrii), Shigella sonnei, Campylobacter concisus, Vibrio sp. (cholerae, parahaemolyticus, vulnificus), Aeromonas sp., Yersinia enterocolitica, and Acinetobacter sp. (including but not limited to Acinebacter baumanii). In other embodiments, the host cell is a plant bacteria (ie: rhizobacteria, endophytic bacteria, Agrobacterium sp. (rhisogens, tumefaciens, vitis), Paracoccus denitrificans, or other bacteria such as Bacillus cereus, Xenorhabdus nematophilus, Micrococcus luteus, Staphylococcus, Xanthomas campestris, Francisella novicida, Rhodobacter sphaeroides, Acidovorax temperans, etc.

A number of T6SS-containing bacteria are known to be associated with human disease, and thus in a preferred embodiment where any of these bacterial types is the host cell, the host cells are attenuated to reduce/eliminate risks associated with use of the bacteria. In a further embodiment, the host cell is a recombinant host cell engineered to express a heterologous (not naturally occurring in the cell type) T6SS. As used herein, a “recombinant T6SS” includes at least 5 of the 13 conserved T6SS “core component” genes, exemplified by those disclosed in Schwarz et al PLoS Pathogens 2010: COG0542 (exemplified by ClpV as disclosed herein), COG3157 (exemplified by Hcp as disclosed herein), COG3455, COG3501 (exemplified by VgrG as disclosed herein), COG3515, COG3516 (exemplified by VipA as disclosed herein), COG3517 (exemplified by VipB as disclosed herein), COG3518, COG3519, COG3520, COG3521, COG3522, COG3523 (exemplified by IcmF as disclosed herein). Sequences and other information can be found, for example at the Genome Reviews web site (ftp://ftp.ebi.ac.uk/pub/databases/genome_reviews/). The recombinant T6SS can comprise or consist of 5, 6, 7, 8, 9, 10, 11, 12, or all 13 of the recited T6SS “core component” genes. In a preferred embodiment, COG3516 (exemplified by VipA protein) is one of the TG66 core component genes used to construct a recombinant T6SS; in further embodiments the TG66 core component genes used to construct a recombinant T6SS further comprise 1, 2, 3, 4, or all 5 of COG0542 (exemplified by ClpV as disclosed herein), COG3157 (exemplified by Hcp as disclosed herein), COG3501 (exemplified by VgrG as disclosed herein), COG3516 (exemplified by VipA as disclosed herein), and COG3517 (exemplified by VipB as disclosed herein).

Exemplary references that describe the entire T6SS for a number of different organisms include Boyer F and Attree I, BMC Genomics 2009 Mar. 12; 10:104; and Schwarz, et al. (2010) PLoS Pathog 6(8): e1001068. doi:10.1371/journal.ppat.1001068.

Existing bacteria or recombinant host cells engineered to express a heterologous T6SS can be tested for T6SS activity by, for example, hemolysin co-regulated protein (Hcp) and/or VgrG secretion. In another embodiment, a functional T6SS that targets a bacterial cell can be detected by comparing the fitness of the bacterium the T6SS is expressed in against a target bacterium relative to the fitness against that target bacterium if the T6SS is disabled by the removal of one of the essential genes (including icmF (also called tssM) or clpV).

The host cells of this aspect of the invention comprise a recombinant gene encoding a therapeutic polypeptide. The therapeutic polypeptide can be any polypeptide that can be secreted by the T6SS in the recombinant cells and provide a therapeutic benefit. As disclosed below, the inventors have found that the T6SS pathway is of general significance to interbacterial interactions in polymicrobial human diseases and the environment, and thus the host cells of this aspect of the invention can be used for targeting toxins (or other proteins/macromolecules) to bacteria involved in disease (human, animal, plant) and environmental contamination. In one embodiment, the therapeutic polypeptide is toxic to bacteria, including but not limited to bactericidal proteins group IIA phospholipase A2, bactericidal/permeability-increasing protein, human peptidoglycan recognition proteins 3 and 4 (PGLYRP3 and PGLYRP4), Tse1, Tse2, Tse3, or other native T6SS substrates, or functional equivalents thereof. In a preferred embodiment, the therapeutic polypeptide comprises Tse1, Tse2, Tse3, or functional equivalents thereof; all embodiments of Tse1, Tse2, and Tse3 disclosed herein can be used in this aspect of the invention.

Regulatory sequences to direct expression of the recombinant gene can be any suitable for use in the host cell and that are appropriate for a given use. Regulatory sequences are discussed above, and this sixth aspect includes all embodiments of the regulatory sequences and control elements disclosed herein.

In a further preferred embodiment, the recombinant gene encoding a therapeutic polypeptide encodes a fusion polypeptide of the therapeutic polypeptide and one or both of a VgrG polypeptide and a Hcp polypeptide. Bacteria expressing T6SSs have been demonstrated to secrete Hcp and VgrG, and have been shown to secrete variants of these conserved proteins that include additional peptide domains (Blondel et al., BMC Genomics 2009, 10:354). Thus, in this embodiment the host cells are engineered such that a VgrG polypeptide or a Hcp polypeptide is utilized to secrete the therapeutic polypeptide through the T6SS to, for example, inhibit bacteria involved in disease (human, animal, plant) or environmental contamination. In one preferred embodiment, the VgrG polypeptide and/or the Hcp polypeptide used are natural substrates of the T6SS being used, in that they are derived from the same organism as the T6SS is derived from. In one exemplary embodiment, the Hcp polypeptide comprises or consists of a P. aeruginosa Hcp polypeptide comprising or consisting of the amino acid sequence of SEQ ID NO:50, or functional fragment thereof. In another exemplary embodiment, the VgrG polypeptide comprises or consists of a P. aeruginosa VgrG polypeptide comprising or consisting of the amino acid sequence of SEQ ID NO:52, or functional fragment thereof.

Thus, the present invention also provides novel fusion polypeptides comprising (a) a therapeutic polypeptide selected from the group consisting of bactericidal proteins group HA phospholipase A2, bactericidal/permeability-increasing protein, human peptidoglycan recognition proteins 3 and 4 (PGLYRP3 and PGLYRP4), Tse1, Tse2, Tse3, or other native T6SS substrates, or functional equivalents thereof; and (b) one or both of a VgrG polypeptide and a Hcp polypeptide, and novel genes encoding such polypeptides. In a preferred embodiment, the therapeutic polypeptide comprises Tse1, Tse2, Tse3, or functional equivalents thereof; all embodiments of Tse1, Tse2, and Tse3 disclosed herein can be used in this aspect of the invention. The recombinant genes as described in this aspect can be used, for example, in vectors for transfection to create the host cells of the sixth aspect of the invention. The invention further comprises novel fusion proteins comprising (a) a therapeutic polypeptide selected from the group consisting of bactericidal proteins group IIA phospholipase A2, bactericidal/permeability-increasing protein, human peptidoglycan recognition proteins 3 and 4 (PGLYRP3 and PGLYRP4), Tse1, Tse2, Tse3, or other native T6SS substrates, or functional equivalents thereof; and (b) one or both of a VgrG polypeptide and a Hcp polypeptide. In a preferred embodiment, the therapeutic polypeptide comprises Tse1, Tse2, Tse3, or functional equivalents thereof; all embodiments of Tse1, Tse2, and Tse3 disclosed herein can be used in this aspect of the invention.

In a seventh aspect, the present invention provides pharmaceutical compositions comprising (a) the recombinant host cells of the sixth aspect of the invention; and (b) a pharmaceutically acceptable carrier. Any suitable carrier can be used for a given application. The compositions of the invention may be used for in vivo applications, such as the therapeutic aspects of the invention disclosed below. For in vivo use, the composition may be formulated for delivery via standard administrative routes, or may be administered, for example, as part of a fermented food product (“probiotic”), such as yogurt, beverage, or a dietary supplement.

In an eighth aspect, the present invention provides an anti-bacterial composition comprising a Tse-expressing recombinant host cell or a Tse polypeptide of any embodiment disclosed above adhered to a substrate. This embodiment can be any type of composition that permits cell-cell contact between the cells of the composition and bacterial cells to be eliminated. Such compositions include, but are not limited to, liquids, soaps, wipes, powders, etc. The anti-bacterial composition can contain any other anti-bacterial or other components as suitable for a given purpose.

In a ninth aspect, the present invention provides methods for inhibiting bacterial growth, comprising contacting bacteria to be inhibited with an amount of the Tse-expressing host cells of any embodiment of the invention or the substantially purified Tse polypeptides of any embodiment of the invention effective to inhibit bacterial growth. As disclosed below, the inventors have found that the T6SS pathway is of general significance to interbacterial interactions in polymicrobial human diseases and the environment, and thus the host cells of the invention can be used for targeting toxins (or other proteins/macromolecules) to bacteria involved in disease (human, animal, plant) and environmental contamination. The inventors have further identified three different toxins (Tse1, Tse2, and Tse3) that are toxic to bacteria, and thus these toxins can be administered to inhibit bacterial growth. Thus, the methods may comprise (a) contacting of the Tse-polypeptide or Tse polypeptide-containing pharmaceutical composition to a subject with a bacterial infection; (b) contacting of the Tse-polypeptide or Tse polypeptide-containing pharmaceutical composition with a plant to be treated; or (c) contacting of the Tse polypeptide or Tse polypeptide-containing pharmaceutical composition to a surface to be treated.

As used herein, “inhibiting bacterial growth” includes one or more of slowing the growth rate of bacteria, minimizing further bacterial replication, and killing of existing bacteria. The bacteria may be gram negative bacteria or gram positive bacteria. In one preferred embodiment, the method comprises in vivo administration of the composition or polypeptide to a subject with a bacterial infection. The “subject” can be a human, animal (cattle, dogs, cats, sheep, chickens, etc.), or plant. The bacterial infection can be any one caused by bacteria susceptible to growth inhibition by the therapeutic. The methods can be used for treatment of diseases linked to bacterial infection, including but not limited to gingivitis, middle ear infections, myocarditis, pneumonia, urinary tract/GI infections, and infections associated with burn victims, cystic fibrosis, and plant bacterial infections. In a preferred embodiment, the bacteria to be inhibited are present in a biofilm. A biofilm is an aggregate of microorganisms in which cells adhere to each other and/or to a surface. Nearly every species of microorganism, not only bacteria and archaea, have mechanisms by which they can adhere to surfaces and to each other. Biofilms will form on virtually every non-shedding surface in a non-sterile aqueous environment. In another preferred embodiment the method comprises administration of the composition or the polypeptide to a surface to be treated. Any surface that is subject to bacterial contamination (such as biofilm formation) can be contacted, including but not limited to medical devices (ex: catheters, contact lens, heart valves, joint prostheses, intrauterine devices, etc.) countertops, door handles, sinks, faucets, showers, water and sewage pipes, floors, pipelines (ex: oil and gas pipelines), boat hulls, teeth, infected skin wounds, plants, etc.

In a tenth aspect, the present invention provides methods for inhibiting cell (eukaryotic or prokaryotic) growth, comprising contacting cells to be inhibited with an amount of the substantially purified Tse protein conjugates of the first aspect of the invention effective to inhibit eukaryotic cell growth. As disclosed below, the Tse2 protein is toxic to cells (prokaryotic and eukaryotic) when expressed intracellularly, while Tse1 and Tse3 have potent antibacterial activity when present periplasmically. In one embodiment, the conjugate comprises a Tse2-transduction domain conjugate. Transduction domains can be linked, for example, to other polypeptides to direct movement of the linked polypeptide across cell membranes. Thus, the Tse-transduction fusion proteins can be used to directly administer the Tse toxins to deleterious cells. In other embodiments for antibacterial use, the Tse protein comprises a conjugate as discussed above, comprising the Tse protein and one or more of the following:

(a) a targeting domain to carry the conjugate across a bacterial outer membrane to a periplasmic space, including but not limited to colicin (or domains thereof) and liposomes;

(b) a phage capsid;

(c) a leader sequence to target intracellular Tse protein to the periplasmic space, including but not limited to PelB, or domains thereof.

In a preferred embodiment, the eukaryotic cell is a mammalian cell; more preferably a human cell. The Tse2-based methods can be used to treat any diseases associated with undesired cell growth, including but not limited to, cancer, diabetic retinopathy, psoriasis, and rheumatoid arthritis. The substantially purified polypeptides may be used alone or in combination with any other suitable therapeutics for inhibiting eukaryotic cell growth.

In practicing these various methods of the invention, the amount or dosage range of the proteins, conjugates, or pharmaceutical compositions employed is one that effectively inhibits bacterial or eukaryotic cell growth. Such an inhibiting amount of the polypeptides or host cells will vary depending on the disorder being treated and other factors, and can be determined by one of skill in the art. For human therapeutic use, in one non-limiting embodiment the Tse proteins or conjugates thereof is administered at a dosage of between about 1 ng/kg and about 10 mg/kg.

In an eleventh aspect, the present invention provides recombinant vectors, comprising a first gene coding for Tse1 or Tse3, of functional equivalents thereof, wherein the first gene is operatively linked to a heterologous regulatory sequence. As disclosed herein, Tse1 and Tse3 have antibacterial activity. Thus, Tse1 and Tse3 can be used, for example, in negative selection cloning in bacterial cells, such as gram (+) or gram (−) bacteria. Tse1 and Tse3 can also be used when selection using an antibiotic is not suitable to the experiment design.

As used herein, a “gene” is any nucleic acid capable of expressing the recited protein, and thus includes genomic DNA, mRNA, cDNA, etc.

In one preferred embodiment, the first gene comprises or consists of a nucleotide sequence that encode a P. aeruginosa Tse1 or Tse3 amino acid sequence according to SEQ ID NO:10 or SEQ ID NO:12. In another preferred embodiment, the first gene comprises or consists of a nucleotide sequence according to SEQ ID NO:9 or SEQ ID NO:11.

As used herein, “Tse1” and “Tse3” includes functional equivalents (truncations, mutants, etc.) thereof, wherein such equivalents maintain antibacterial activity as described herein. Methods for identifying such functional equivalents are disclosed herein.

In a further preferred embodiment of any embodiment disclosed above, the first gene encodes a Tse 1 or Tse3 conjugate. As disclosed below, Tse1 and Tse3 proteins are toxic to bacteria when present periplasmically.

In another embodiment, the first gene encodes a Tse1 or Tse3 fusion protein comprising a secretory signal sequence, permitting secretion of the fusion protein to the periplasmic space. Any suitable secretory signal sequence can be used, as are known in the art. The coding region for the secretory peptide may be in any suitable relationship relative to the Tse1 or Tse3 coding sequence, such as positioned to encode the amino terminus of the fusion protein. The coding region can also be designed to permit cleavage of the secretory signal sequence.

The regulatory sequence is “heterologous”, meaning that it is not a naturally occurring Tse1 of Tse3 regulatory region. As used herein, “regulatory sequence” and “promoter” are as defined above.

In one embodiment, the Tse1 or Tse3 gene is operatively linked to a promoter element sufficient to render promoter-dependent controllable gene expression, for example, inducible or repressible by external signals or agents (adding/removing compounds from the growth media for the recombinant cells), or by altering culture conditions (temperature, pH, etc.). Exemplary controllable promoters are those that are alcohol-regulated, tetracycline-regulated, steroid-regulated, metal-regulated, pathogen-regulated, light-regulated, or temperature-regulated. For use in bacterial systems, many controllable promoters are known (Old and Primrose, 1994). Common examples include P_{lac} (IPTG), P_{tac} (IPTG), lambdaP_{R} (loss of CI repressor), lambdaP_{L} (loss of CI repressor), P_{trc} (IPTG), Pₜᵣₚ (IAA). The controlling agent is shown in brackets after each promoter. Examples of controllable plant promoters include the root-specific ANRI promoter (Zhang and Forde (1998) Science 279:407) and the photosynthetic organ-specific RBCS promoter (Khoudi et al. (1997) Gene 197:343). Further exemplary controllable promoters include the Tet-system (Gossen and Bujard, PNAS USA 89: 5547-5551, 1992), the ecdysone system (No et al., PNAS USA 93: 3346-3351, 1996), the progesterone-system (Wang et al., Nat. Biotech 15: 239-243, 1997), and the rapamycin-system (Ye et al., Science 283:88-91, 1999), arabinose-inducible promoters, and rhamnose-inducible promoters.

In accordance with the invention, any vector may be used to construct the vectors of invention. In particular, vectors known in the art and those commercially available (and variants or derivatives thereof) may in accordance with the invention be engineered to include one or more nucleic acid molecules encoding one or more recombination sites (or portions thereof), or mutants, fragments, or derivatives thereof, for use in the methods of the invention. Such vectors may be obtained from, for example, Vector Laboratories Inc.; Promega; Novagen; New England Biolabs; Clontech; Roche; Pharmacia; EpiCenter; OriGenes Technologies Inc.; Stratagene; Perkin Elmer; Pharmingen; and Invitrogen Corp., Carlsbad, Calif. Such vectors may then for example be used for cloning or subcloning nucleic acid molecules of interest. General classes of vectors of particular interest include prokaryotic and/or eukaryotic cloning vectors, Expression Vectors, fusion vectors, two-hybrid or reverse two-hybrid vectors, shuttle vectors for use in different hosts, mutagenesis vectors, transcription vectors, and the like.

Other vectors of interest include viral origin vectors (M13 vectors, bacterial phage λ. vectors, bacteriophage P1 vectors, adenovirus vectors, herpesvirus vectors, retrovirus vectors, phage display vectors, combinatorial library vectors), high, low, and adjustable copy number vectors, and vectors which have compatible replicons for use in combination in a single host (pACYC184 and pBR322.

Other vectors of particular interest include pUC18, pUC19, pBlueScript, pSPORT, cosmids, phagemids, BACs (bacterial artificial chromosomes), pQE70, pQE60, pQE9 (Quiagen), pBS vectors, PhageScript vectors, BlueScript vectors, pNH8A, pNH16A, pNH18A, pNH46A (Stratagene), pcDNA3 (Invitrogen, Carlsbad, Calif.), pGEX, pTrsfus, pTrc99A, pET-5, pET-9, pKK223-3, pKK233-3, pDR540, pRIT5 (Pharmacia), pSPORT1, pSPORT2, pCMVSPORT2.0 and pSV-SPORT1 (Invitrogen Corp., Carlsbad, Calif.) and variants or derivatives thereof.

Additional vectors of interest include pTrxFus, pThioHis, pLEX, pTrcHis, pTrcHis2, pRSET, pBlueBacHis2, pcDNA3.1/His, pcDNA3.1(−)/Myc-His, pSecTag, pEBVHis, pPIC9K, pPIC3.5K, pAO815, pPICZ, pGAPZ, pBlueBac4.5, pBlueBacHis2, pMelBac, pSinRep5, pSinHis, pIND, pIND(SP1), pVgRXR, pcDNA2.1. pYES2, pZErO1.1, pZErO-2.1, pCR-Blunt, pSE280, pSE380, pSE420, pVL1392, pVL1393, pCDM8, pcDNA1.1, pcDNA1.1/Amp, pcDNA3.1, pcDNA3.1/Zeo, pSe,SV2, pRc/CMV2, pRc/RSV, pREP4, pREP7, pREP8, pREP9, pREP10, pCEP4, pEBVHis, pCR3.1, pCR2.1, pCR3.1-Uni, and pCRBac from Invitrogen; λgt11, pTrc99A, pKK223-3, pGEX-2T, pGEX-2TK, pGEX-4T-1, pGEX-4T-2, pGEX-4T-3, pGEX-3X, pGEX-5XX1, pGEX-5XX2, pGEX-5X-3, pEZZ18, pRIT2T, pMC1871, pSVK3, pSVL, pMSG, pCH110, pKK232-8, pSL1180, pNEO, and pUC4K from Pharmacia; pSCREEN-Ib(+), pT7Blue(R), pT7Blue-2, pCITE-4-abc(+), pOCUS-2, pTAg, pET-32 LIC, pET-30 LIC, pBAC-2 cp LIC, pBACgus-2 cp LIC, pT7Blue-2 LIC, pT7Blue-2, pET-3abcd, pET-7abc, pET9abcd, pET11abcd, pET12abc, pET-14b, pET-15b, pET-16b, pET-17b-pET-17xb, pET-19b, pET-20b(+), pET-21abcd(+), pET-22b(+), pET-23abcd(+), pET-24abcd(+), pET-25b(+), pET-26b(+), pET-27b(+), pET-28abc(+), pET-29abc(+), pET-30abc(+), pET-31b(+), pET-32abc(+), pET-33b(+), pBAC-1, pBACgus-1, pBAC4x-1, pBACgus4x-1, pBAC-3 cp, pBACgus-2 cp, pBACsurf-1, plg, Signal plg, pYX, Selecta Vecta-Neo, Selecta Vecta-Hyg, and Selecta Vecta-Gpt from Novagen; pLexA, pB42AD, pG13T9, pAS2-1, pGAD424, pACT2, pGAD GL, pGAD GH, pGAD10, pGilda, pEZM3, pEGFP, pEGFP-1, pEGFP-N, pEGFP-C, pEBFP, pGFPuv, pGFP, p6xHis-GFP, pSEAP2-Basic, pSEAP2-Contral, pSEAP2-Promoter, pSEAP2-Enhancer, pβgal-Basic, pβgal-Control, pβgal-Promoter, pβgal-Enhancer, pTet-Off, pTet-On, pTK-Hyg, pRetro-Off, pRetro-On, pIRESlneo, pIRES1hyg, pLXSN, pLNCX, pLAPSN, pMAMneo, pMAMneo-CAT, pMAMneo-LUC, pPUR, pSV2neo, pYEX 4T-1/2/3, pYEX-S1, pBacPAK-His, pBacPAK8/9, pAcUW31, BacPAK6, pTriplEx, .lamda.gt10, .lamda.gt11, and pWE15, and from Clontech; Lambda ZAP II, pBK-CMV, pBK-RSV, pBluescript II KS +/−, pBluescript II SK +/−, pAD-GAL4, pBD-GAL4 Cam, pSurfscript, Lambda FIX II, Lambda DASH, Lambda EMBL3, Lambda EMBL4, SuperCos, pCR-Scrigt Amp, pCR-Script Cam, pCR-Script Direct, pBS +/−, pBC KS +/−, pBC SK +/−, Phagescript, pCAL-n-EK, pCAL-n, pCAL-c, pCAL-kc, pET-3abcd, pET-11abcd, pSPUTK, pESP-1, pCMVLacI, pOPRSVI/MCS, pOPI3 CAT, pXT1, pSG5, pPbac, pMbac, pMClneo, pMClneo Poly A, pOG44, p0045, pFRTβGAL, pNEOβGAL, pRS403, pRS404, pRS405, pRS406, pRS413, pRS414, pRS415, and pRS416 from Stratagene.

Vectors according to this aspect of the invention include, but are not limited to: pENTR1A, pENTR2B, pENTR3c, pENTR4, pENTR5, pENTR6, pENTR7, pENTR8, pENTR9, pENTR10, pENTR11, pDEST1, pDEST2, pDEST3, pDEST4, pDEST5, pDEST6, pDEST7, pDEST8, pDEST9, pDEST10, pDEST11, pDEST12.2 (also known as pDEST12), pDEST13, pDEST14, pDEST15, pDEST16, pDEST17, pDEST18, pDEST19, pDEST20, pDEST21, pDEST22, pDEST23, pDEST24, pDEST25, pDEST26, pDEST27, pEXP501 (also known as pCMVSPORT6.0), pDONR201, pDONR202, pDONR203, pDONR204, pDONR205, pDONR206, pDONR212, pDONR212(F) (FIGS. 28A-28C), pDONR212(R) (FIGS. 29A-29C), pMAB58, pMAB62, pDEST28, pDEST29, pDEST30, pDEST31, pDEST32, pDEST33, pDEST34, pDONR207, pMAB85, pMAB86, a number of which are described in PCT Publication WO 00/52027 (the entire disclosure of which is incorporated herein by reference), and fragments, mutants, variants, and derivatives of each of these vectors. However, it will be understood by one of ordinary skill that the present invention also encompasses other vectors not specifically designated herein, which comprise one or more of the isolated nucleic acid molecules used in the invention encoding one or more recombination sites or portions thereof (or mutants, fragments, variants or derivatives thereof), and which may further comprise one or more additional physical or functional nucleotide sequences described herein which may optionally be operably linked to the one or more nucleic acid molecules encoding one or more recombination sites or portions thereof. Such additional vectors may be produced by one of ordinary skill according to the guidance provided in the present specification.

Expression vectors and methods for their engineering and isolation are well known in the art (see, e.g., Maniatis et al., supra), or they can be obtained through a commercial vendor, e.g., Invitrogen (Carlsbad, Calif.), Promega (Madison, Wis.), and Statagene (La Jolla, Calif.) and modified as needed. Examples of commercially available expression vectors include pcDNA3 (Invitrogen), Gateway cloning technology (Life Technologies), and pCMV-Script (Stratagene). Vector components, regulatory nucleic acids, etc. are typically available from a commercial source or can be isolated from a natural source (e.g., animal tissue or microorganism) or prepared using a synthetic means such as PCR. The arrangement of the components can be any arrangement practically desired by one of ordinary skill in the art. Vectors used in the present invention can be derived from viral genomes that yield virions or virus-like particles, which may or may not replicate independently as extrachromosomal elements. Virion particles can be introduced into the host cells by infection. The viral vector may become integrated into the cellular genome. Examples of viral vectors for transformation of mammalian cells are SV40 vectors, and vectors based on papillomavirus, adenovirus, Epstein-Barr virus, vaccinia virus, and retroviruses, such as Rous sarcoma virus, or a mouse leukemia virus, such as Moloney murine leukemia virus. For mammalian cells, electroporation or viral-mediated introduction can be used.

In one embodiment, the vector comprises one or more unique restriction enzyme recognition sites, wherein cloning of a nucleic acid insert into the one or more unique restriction enzyme recognition sites disrupts expression of Tse1 and/or Tse3. The vectors of this embodiment can be used as cloning vehicles, since cloning of an insert into the one or more restriction sites in the vector interrupts Tse1 and/or Tse3 expression and provide an easily selectable marker—cells with vectors containing no insert have their growth inhibited by Tse1 and/or Tse3 expression, and those with inserts do not. In one preferred embodiment, one or more unique restriction sites are engineered into the coding region for Tse1 and/or Tse3 using techniques well known to those of skill in the art, such that cloning an insert into the restriction site disrupts the coding region for Tse1 and/or Tse3. In this embodiment, the restriction sites can be engineered into the coding region to result in silent nucleotide changes, or may result in one or more changes in the amino acid sequence of Tse1 and/or Tse3, so long as the encoded Tse1 and/or Tse3 protein retains antibacterial activity. Alternatively, the one or more unique restriction sites may be located in regulatory regions such that cloning of an insert would disrupt expression of Tse1 or Tse3 from the vector. Design and synthesis of nucleic acid sequences and preparation of vectors comprising such sequences is well within the level of skill in the art.

In another embodiment, the Tse 1 and/or Tse3 may be encoded in the vector as a fusion protein. In this embodiment, the cloning vector includes at least one promoter nucleotide sequence and at least one nucleotide sequence encoding a fusion protein (Tse1 and/or Tse3) which is active as a poison, the nucleotide sequence being obtained by fusing a gene coding nucleotide sequence which includes multiple unique cloning sites (MCS) and a nucleotide sequence which encodes Tse1 and/or Tse3. An analogous system utilizing the prokaryotic death gene ccdB has been described in U.S. Pat. No. 7,176,029, and is incorporated by reference herein in its entirety. Exemplary fusion protein comprise, but are not limited to, lacZα, GFP, RFP, H is, TA fusion proteins with Tse1 and/or Tse3.

In one non-limiting embodiment, the cloning vector contains the Tse1 and/or Tse3 gene fused to the C-terminus or N-terminus of LacZα. The expression of the Tse1 and/or Tse3-LacZ fusion protein is controlled by an inducible promoter, such as the lac promoter, such that expression of the Tse1 and/or Tse3-LacZ fusion protein will result in the death of a cell. In certain embodiments, a multiple cloning site (MCS) is contained within the LacZ gene, such that insertion of a DNA fragment disrupts the expression of the lacZα-Tse1 and/or Tse3 gene fusion, permitting growth of only positive recombinants. Cells that contain nonrecombinant vector are killed. Plasmids according to this embodiment allow doubly digested restriction fragments to be cloned in both orientations with respect to the lac promoter. Insertion of a restriction fragment into one of the unique cloning sites interrupts the genetic information of the gene fusion, leading to the synthesis of a gene fusion product which is not functional. Insertional inactivation of the gene fusion ought always to take place when a termination codon is introduced or when a change is made in the reading frame. The cells which harbor a recombinant vector (disrupted Tse1 and/or Tse3) will be viable while cells which harbor an intact vector (intact Tse1 and/or Tse3) will not be viable. This negative selection, by simple culture on a solid medium, makes it possible to eliminate cells which harbor a non-recombinant vector (non-viable clones) and to select recombinant clones (viable clones).

In another embodiment, the recombinant vector comprises one or more recombination sites flanking the Tse1 and/or Tse3 gene. In a preferred embodiment, the recombinant vector comprises at least a first and a second recombination site flanking a first gene coding for Tse1 and/or Tse3 operatively linked to a regulatory sequence, wherein said first and second recombination sites do not recombine with each other. As used herein, a “recombination site” is a discrete section or segment of DNA that is recognized and bound by a site-specific recombination protein during the initial stages of integration or recombination. For example, the recombination site for Cre recombinase is loxP, a 34 base pair sequence comprised of two 13 base pair inverted repeats (serving as the recombinase binding sites) flanking an 8 base pair core sequence. See Sauer, B., Curr. Opin. Biotech. 5:521-527 (1994). Other examples of recognition sequences include the attB, attP, attL, and attR sequences which are recognized by the recombination protein λattB is an approximately 25 base pair sequence containing two 9 base pair core-type Int binding sites and a 7 base pair overlap region, while attP is an approximately 240 base pair sequence containing core-type Int binding sites and arm-type Int binding sites as well as sites for auxiliary proteins integration host factor (IHF), FIS and excisionase (Xis). See Landy, Curr. Opin. Biotech. 3:699 707 (1993). Further examples of recognition sequences include loxP site mutants, variants or derivatives such as loxP511 (see U.S. Pat. No. 5,851,808); dif sites; dif site mutants, variants or derivatives; psi sites; psi site mutants, variants or derivatives; cer sites; and cer site mutants, variants or derivatives. See also, for example, US20100267128 and WO 01/11058, incorporated by reference herein in their entirety. Other systems providing recombination sites and recombination proteins for use in the invention include the FLP/FRT system from Saccharomyces cerevisiae, the resolvase family (e.g., RuvC, yi, TndX, TnpX, Tn3 resolvase, Hin, Hjc, Gin, SpCCE1, ParA, and Cin), and IS231 and other Bacillus thuringiensis transposable elements. Other suitable recombination systems for use in the present invention include the XerC and XerD recombinases and the psi, dif and cer recombination sites in Escherchia coli. Other suitable recombination sites may be found in U.S. Pat. No. 5,851,808, which is specifically incorporated herein by reference.

This embodiment can be used for recombinational cloning, for example using the system described in published U.S. Pat. Application No. US20100267128, and in U.S. application Ser. No. 09/177,387, filed Oct. 23, 1998; U.S. application Ser. No. 09/517,466, filed Mar. 2, 2000; and U.S. Pat. Nos. 5,888,732 and 6,143,557, all of which are specifically incorporated herein by reference. In brief, the disclosed system utilizes vectors that contain at least two different site-specific recombination sites based on the bacteriophage lambda system (e.g., att1 and att2) that are mutated from the wild-type (att0) sites. Each mutated site has a unique specificity for its cognate partner att site (i.e., its binding partner recombination site) of the same type (for example attB1 with attP 1, or attL1 with attR1) and will not cross-react with recombination sites of the other mutant type or with the wild-type att0 site. Different site specificities allow directional cloning or linkage of desired molecules thus providing desired orientation of the cloned molecules. Nucleic acid fragments flanked by recombination sites are cloned and subcloned by replacing a selectable marker (Tse1 and/or Tse3) flanked by att sites on the recipient plasmid molecule. Desired clones are then selected by transformation of a Tse1 and/or Tse3 sensitive host strain and positive selection for a marker on the recipient molecule. Tse1 and/or Tse3 is toxic to both bacterial cells, and thus Tse1 and/or Tse3 sensitive host strains include bacterial cells, including gram (+) and gram (−) bacteria.

In one embodiment, the vector contains a Tse1 and/or Tse3 gene flanked by one or more restriction enzyme sites or recombination sites. Recombination sites include, but are not limited to, attB, attP, attL, and attR. This vector is designed such that the DNA fragment of interest (such as, for example, a PCR product) will replace the Tse1 and/or Tse3 located between the two flanking sites. If the DNA fragment of interest is present in the vector, the cells containing the vector survive, as the Tse1 and/or Tse3 gene will no longer be present on the desired recombinant vector. If the gene of interest is not present, the Tse1 and/or Tse3 gene will prevent survival of the cell carrying the undesired vector. Thus, only cells containing positive clones with the DNA fragment of interest will be viable, and easily selected for.

In one embodiment, the vector comprises at least one inactive fragment of the Tse1 and/or Tse3 gene, wherein a functional Tse1 and/or Tse3 gene is rescued when the inactive fragment is recombined across at least one recombination site with a second DNA segment comprising another inactive fragment of the Tse1 and/or Tse3 gene.

In another embodiment, the vector contains a dual selection cassette, wherein the vector comprises a first gene coding for Tse1 and/or Tse3, and a second gene encoding a second selectable marker, such as an antibiotic resistance gene or a second “death” gene encoding a second toxic protein. The antibiotic resistance gene can be selected from either bacterial or eukaryotic genes, and can promote resistance to ampicillin, kanamycin, tetracycline, cloramphenicol, and others known in the art. The second death gene can be any suitable death gene, including but not limited to the combination of Tse1 with Tse3; rpsL, tetAR, pheS, thyA, lacY, gata-1, ccdB, and sacB. The second death gene can also be selected from either prokaryotic or eukaryotic toxic genes. This dual selection cassette is flanked by at least one restriction site or recombination site, such that the DNA fragment of interest will replace the dual selection cassette located between the two sites in the desired recombination or ligation event. If the DNA fragment of interest is present, the cells containing the vector survive, as the Tse1 and/or Tse3 gene will no longer be present on the desired recombinant vector. If the gene of interest is not present, the vector will still contain the Tse1 and/or Tse3 gene and will prevent survival of the cell carrying the undesired vector. This dual selection cassette can thus be used for any double negative selection strategy as desired by one of ordinary skill in the art. In one embodiment, the Tse1 and/or Tse3 gene double negative selection strategy is used when use of multiple antibiotics is not compatible with the particular selection design.

As a non-limiting example, the vector contains a dual selection cassette comprising the Tse1 and/or Tse3 gene as well as a cloramphenicol resistance gene under control of at least one promoter. The vector is cut using restriction enzymes both upstream and downstream of the dual selection cassette. Optionally, the linearized vector can be gel purified to remove the excised dual selection cassette DNA from the reaction. DNA containing the DNA fragment of interest and appropriate restriction enzyme sites, such as a PCR product, is then combined with the linearized vector in a ligation reaction. Positive clones will be chloramphenicol sensitive and viable (Tse1 and/or Tse3 gene negative), due to the replacement of the dual selection cassette with the DNA fragment of interest.

In another embodiment, the vector contains at least one recombination site within the Tse1 and/or Tse3 gene or corresponding regulatory element (e.g. promoter or enhancer), such that a desired recombination event will disrupt the expression of the Tse1 and/or Tse3 gene from the vector. The location of the recombination site should be chosen such that if the desired recombination event occurs, the resulting Tse1 and/or Tse3 gene will be inactive and the cell containing the desired vector will survive. If the desired recombination event does not occur, the Tse1 and/or Tse3 gene will remain intact and the cell containing the undesired vector will not survive.

In another embodiment, the vector contains at least one recombination site within the Tse1 and/or Tse3 gene or corresponding regulatory element (e.g. promoter or enhancer), such that an undesired recombination event will produce an intact and functional Tse1 and/or Tse3 gene, which will result in the death of the cell containing the undesired vector.

In another embodiment, the Tse1 and/or Tse3 gene is fragmented on multiple vectors, with shared restriction enzyme sequences or recombination site sequences connecting the gene fragments. The vectors are designed and arranged such that an undesired recombination event or ligation event will result in the creation of an intact Tse1 and/or Tse3 gene on the undesired plasmid, thus resulting in the death of the cells containing the undesired vector with the functional Tse1 or Tse3 gene.

In another embodiment, the vectors are ones suitable for topoisomerase-mediated cloning, as described in U.S. Pat. Nos. 5,766,891 and 7,550,295, and/or TA cloning, as disclosed in U.S. Pat. No. 5,827,657, both references incorporated by reference herein in their entirety. In certain embodiments, the vectors suitable for topoisomerase or TA-mediated cloning are linearized, such that the vectors are optimized for most efficient integration of the DNA fragment of interest. These preparations are described in the referenced patents.

Briefly, topoisomerase-mediated cloning relies on the principle that Taq polymerase has a non-template-dependent terminal transferase activity that adds a single deoxyadenosine (A) to the 3′ ends of PCR products. For example, topoisomerase I from Vaccinia virus binds to duplex DNA at specific sites (CCCTT) and cleaves the phosphodiester backbone in one strand. The energy from the broken phosphodiester backbone is conserved by formation of a covalent bond between the 3′ phosphate of the cleaved strand and a tyrosyl residue (Tyr-274) of topoisomerase I. The phospho-tyrosyl bond between the DNA and enzyme can subsequently be attacked by the 5′ hydroxyl of the original cleaved strand, reversing the reaction and releasing topoisomerase. In one embodiment, the vectors of the invention comprise a linear vector containing single, overhanging 3′ deoxythymidine (T) residues, with a topoisomerase I covalently bound to the vector (referred to as “activated vector”). This allows PCR inserts to ligate efficiently with the vector.

In another embodiment, the vectors are designed for topoisomerase or TA cloning, such that the topoisomerase or TA cleavage sites are located within the Tse1 and/or Tse3 gene. In this embodiment, the vector can be used for negative selection of clones that are lacking a desired DNA insert. After conducting the topoisomerase or TA reaction, the vectors that contain a desired DNA insert will have a disrupted and inactive Tse1 and/or Tse3 gene, thus allowing the cells containing that vector to survive. However, if the vector circularizes at the cleavage sites without incorporating an insert, the Tse1 and/or Tse3 gene will be reformed and active, thus producing the toxic Tse1 and/or Tse3 protein and killing the cell. In further embodiments, the topoisomerase or TA site will be flanked with restriction enzyme sites and/or sequencing primer sites.

In another embodiment, the TA or TOPO cloning strategies can be combined, as disclosed, for example, in U.S. Pat. No. 6,916,632, incorporated herein for reference in its entirety.

In another aspect of the invention that can be combined with any other embodiment herein, the recombinant vector may comprise a gene encoding a Tse1 and/or Tse3 antidote operatively linked to a regulatory sequence. The antidote can be any expression product capable of interfering with the antibacterial activity of Tse1 and/or Tse3, including but not limited to Tse1 or Tse3 antisense constructs, Tse1 or Tse3-binding aptamers, and Tse1 or Tse3-binding polypeptides. Such vectors can be used, for example, as markers in a cell whose survivability can be conditionally controlled by controlling conditions under which the antidote polypeptide is expressed. In a preferred embodiment that can be combined with any other embodiment herein, the second gene codes for type VI secretion immunity protein 1 or 3 (Tsi1 or Tsi3), disclosed in the examples that follow as an antidote to Tse1 (Tsi1) or Tse3 (Tse3). In one preferred embodiment, the second gene comprises or consists of a nucleotide sequence that can encode a P. aeruginosa Tsi1 (SEQ ID NO:54) or Tsi3 amino acid sequence (SEQ ID NO:56).

As discussed herein, “Tsi1” and “Tsi3” include functional equivalents (truncations, mutants, etc.) thereof, wherein such equivalents maintain their ability to confer immunity upon cells expressing Tse1 or Tse3, respectively. Methods for identifying such functional equivalents are disclosed.

The Tsi1 and/or Tsi3 gene can be under the regulatory control of any promoter desired, including but not limited to those disclosed above for the Tse proteins, such as the various inducible promoters disclosed above, as well as baculovirus polyhedrin, SP6, metallothionein I, Autographa californica nuclear polyhidrosis virus, Semliki Forest virus, Tet, CMV, Gall, Ga110, and T7 promoters.

In one embodiment, the Tsi1 and/or Tsi3 gene is included on a vector which will, when expressed, confer immunity to a cell which is expressing Tse1 and/or Tse3. In a cell line which is expressing Tse1 and/or Tse3 in the absence of Tsi1 and/or Tsi3, the cells will not survive. Also provided herein is the Tsi1 and/or Tsi3 gene under the control of an inducible promoter, as described above. If a Tse1 and/or Tse3-expressing cell receives the vector which expresses the Tsi1 and/or Tsi3 gene, that cell will survive, while such cells that do not express the Tsi1 and/or Tsi3 gene will not survive.

In another embodiment, the Tsi1 and/or Tsi3 gene can be used as a marker for a desired recombination or ligation event. In a non-limiting example, the vector contains a Tsi1 and/or Tsi3 gene flanked by one or more recombination sites. The DNA fragment of interest is inserted into a site on the vector, such that the fragment does not disrupt the Tsi1 and/or Tsi3 gene but is contained within the recombination sites. In another embodiment, a topoisomerase or TA site is included within the flanking sites, but outside the Tsi1 and/or Tsi3 gene, to facilitate DNA fragment insertion. The vector containing the DNA fragment of interest is then combined with a second vector containing matching recombination sites, such that a positive recombination event will move the DNA fragment of interest and the Tsi1 and/or Tsi3 gene into the new vector, which can then be selected for survival in cells expressing Tse1 and/or Tse3. In another non-limiting example, the vector contains a Tsi1 and/or Tsi3 gene flanked by one or more restriction sites. The DNA fragment of interest is inserted into a site on the vector, such that the fragment does not disrupt the Tsi1 and/or Tsi3 gene but is contained within the restriction sites. The vector containing the DNA fragment of interest and a second cloning vector are then digested with one or more restriction enzymes, followed by a ligation reaction. A positive ligation event will move the DNA fragment of interest and the Tsi1 and/or Tsi3 gene into the second cloning vector, which can then be selected for survival in cells expressing Tse1 and/or Tse3 In another embodiment, different antibiotic resistance genes can also be used on the plasmids such that double selection can be employed by one of ordinary skill in the art.

In one embodiment, the vector comprises a Tsi1 and/or Tsi3 gene in an inactive form, such as a truncated form. This vector can be used, for example, in methods for rescuing the activity of the Tsi1 and/or Tsi3 gene such that vectors which contain a functional Tsi1 and/or Tsi3 gene also contain the DNA fragment of interest (as described herein). The functional Tsi1 and/or Tsi3 can be rescued by recombination, integration, or other events or reactions as described herein. Vectors can be readily designed for the particular experiment by one of ordinary skill in the art.

In another aspect of the invention, the invention provides herein a recombinant vector which contains a truncated or inactive version of the antitoxin (Tsi1 and/or Tsi3) gene is present on the vector. In a non-limiting example, the vector may be in linear form. In order to restore the function of the Tsi1 and/or Tsi3 gene, a short sequence of nucleotides are added to the end of the DNA fragment of interest to be cloned. This sequence corresponds to the truncated sequence of the Tsi1 and/or Tsi3 gene, such that this sequence attached to the DNA fragment of interest will bind with the truncated Tsi1 and/or Tsi3 gene, thus restoring an active antitoxin protein able to counteract the action of the Tse1 and/or Tse3 protein. The short sequence is incorporated to the DNA fragment using one modified PCR primer. This system allows for the positive selection of recombinant plasmids only and for the selection of the correct orientation of the cloned fragment in the vector, as only one of the two possible orientations will restore an active Tsi1 and/or Tsi3 gene.

In another embodiment, the truncation of the Tsi1 and/or Tsi3 gene is located within the regions as defined in the invention as required for Tsi1 and/or Tsi3 antidote function.

In another embodiment, the vector containing the truncated, inactive Tsi1 and/or Tsi3 gene is circular.

In another embodiment, the invention provides a recombinant vector, in which a gene encoding Tsi1 and/or Tsi3 would be functional only after proper elimination of an antibiotic resistance gene or additional cell death gene. Any antibiotic resistance gene or additional death gene could be used in this embodiment. In one non-limiting example, the Tsi1 and/or Tsi3 locus is split into two parts on the same plasmid containing a common sequence, and cloned in the 5′ and 3′ regions flanking the kanamycin resistance gene. After digestion at a restriction site located inside the kanamycin resistance gene and transformation of Tse1 or Tse3 expressing cells with linear DNA, a fully functional Tsi1 and/or Tsi3 would assemble through homologous recombination. Only cells containing a recombinant plasmid with a functional Tsi1 and/or Tsi3 can grow upon transformation. For a description of this strategy using the ccdB gene, see Peubez, et al. Microbial Cell Factories 2010, 9:65, which is incorporated by reference.

In another embodiment, the Tsi1 and/or Tsi3 locus is split into two or more parts on two or more plasmids.

In another embodiment, the Tsi1 and/or Tsi3 locus is split into two or more parts on two or more plasmids or integrated into the chromosome of a cell.

In another embodiment, the vector comprises one or more unique restriction enzyme recognition sites, wherein cloning of a nucleic acid insert into the one or more unique restriction enzyme recognition sites disrupts expression of the Tsi 1 and/or Tsi3 antidote gene. The vectors of this embodiment can be used as cloning vehicles, since cloning of an insert into the one or more restriction sites in the vector interrupts Tsi1 or Tsi3 antidote gene expression and provide an easily selectable marker. Cells with vectors containing no insert survive, while those with insert die.

In another embodiment, the invention comprises a first vector that contains the Tse1 and/or Tse3 gene according to any embodiment disclosed herein, and a second vector that contains the Tsi 1 and/or Tsi3 gene according to any embodiment disclosed herein.

In another embodiment, the invention comprises a vector that contains the Tse1 and/or Tse3 gene according to any embodiment disclosed herein, and contains the Tsi1 and/or Tsi3 gene according to any embodiment disclosed herein.

In one embodiment, the vector contains a Tsi1 and/or Tsi3 gene such that loss of the expression of the Tsi1 and/or Tsi3 gene renders the cell non-viable.

In addition to components of the vector which may be required for expression of Tse1 and/or Tse3 (and Tse1 and/or Tse3 antidote, if present), vectors may also include any other suitable control elements, including but not limited to origin of replication, primer sites, e.g., for PCR, transcriptional and/or translational initiation and/or regulation sites, recombinational signals, replicons, other selection markers, antibiotic resistance genes, etc. In one embodiment, the replication sequence renders the vector capable of episomal and chromosomal replication, such that the vector is capable of self-replication as an extrachromosomal unit and of integration into the chromosome, either due to the presence of a translocatable sequence, such as an insertion sequence or transposon, due to substantial homology with a sequence present in the chromosome or due to non-homologous recombinational events. The replication sequence or replicon will be one recognized by the transformed host and is derived from any convenient source, such as from a plasmid, virus, the host cell, e.g., an autonomous replicating segment, by itse1f, or in conjunction with a centromere, or the like. The particular replication sequence is not critical to the subject invention and various sequences may be employed. Conveniently, a replication sequence of a virus can be employed.

In all embodiments, each individual nucleic acid segment may comprise a variety of sequences including, but not limited to sequences suitable for use as primer sites (e.g., sequences for which a primer such as a sequencing primer or amplification primer may hybridize to initiate nucleic acid synthesis, amplification or sequencing), transcription or translation signals or regulatory sequences such as promoters and/or enhancers, ribosomal binding sites, Kozak sequences, start codons, termination signals such as stop codons, origins of replication, recombination sites (or portions thereof), selectable markers, and genes or portions of genes to create protein fusions (e.g., N-terminal or C-terminal) such as GST, GUS, GFP, YFP, CFP, maltose binding protein, 6 histidines (HIS6), epitopes, haptens and the like and combinations thereof. The vectors used for cloning such segments may also comprise these functional sequences (e.g., promoters, primer sites, etc.). After combination of the segments comprising such sequences and optimally the cloning of the sequences into one or more vectors, the molecules may be manipulated in a variety of ways, including sequencing or amplification of the target nucleic acid molecule (i.e., by using at least one of the primer sites introduced by the integration sequence), mutation of the target nucleic acid molecule (i.e., by insertion, deletion or substitution in or on the target nucleic acid molecule), insertion into another molecule by homologous recombination, transcription of the target nucleic acid molecule, and protein expression from the target nucleic acid molecule or portions thereof (i.e., by expression of translation and/or transcription signals contained by the segments and/or vectors). Cloning vectors can be stored in a freezer, refrigerator, liquid nitrogen, or any other methods known to one of ordinary skill in the art.

In a twelfth aspect, the present invention provides recombinant host cells comprising the recombinant vector of any embodiment or combination of embodiments of the eleventh aspect of the invention. A “host,” as the term is used herein, can be any prokaryotic or eukaryotic organism that can be genetically engineered to express heterologous Tse1 or Tse3 including but not limited to bacterial (such as E. coli), algal, fungal (such as yeast), insect, invertebrate, plant, and mammalian cell types. For examples of such hosts, see Maniatis et al., Molecular Cloning: A Laboratory Manual, Cold Spring Harbor Laboratory, Cold Spring Harbor, N.Y. (1982). The host cells of this aspect of the invention can be used, for example, in the methods of the invention discussed below.

In a thirteenth aspect, the present invention provides methods for selectable cloning, comprising culturing the recombinant host cell of any embodiment of the twelfth aspect of the invention under conditions suitable for expression of Tse1 or Tse3 from the recombinant vector if no insert is present, and selecting those cells that grow as comprising recombinant vectors with the insert cloned into the expression vector. In one embodiment, the vector comprises one or more unique restriction enzyme recognition sites, and wherein cloning of a nucleic acid insert into the one or more unique restriction enzyme recognition sites disrupts expression of the first gene, and cloning of an insert into the one or more restriction sites in the vector interrupts Tse1 and/or Tse3 expression and provide an easily selectable marker—cells comprising vectors containing no insert have their growth inhibited by Tse1 and/or Tse3 expression, and those with inserts do not. In another embodiment, the recombinant vector comprises at least a first and a second recombination site flanking a first gene coding for Tse1 and/or Tse3 operatively linked to a regulatory sequence, wherein said first and second recombination sites do not recombine with each other. In this embodiment, nucleic acid fragments to be cloned are flanked by recombination sites and cloned/subcloned by replacing the Tse1 and/or Tse3 selectable marker flanked by recombination sites on the recombinant vector. Desired clones are then selected by transformation of a Tse1 and/or Tse3 sensitive host strain and any positive selection for a marker on the recipient molecule. Tse1 and/or Tse3 have potent antibacterial activity, and Tse1 and/or Tse3 sensitive host strains include both gram (+) and gram (−) bacteria. Conditions for cell culture suitable for Tse1 or Tse3 expression can be determined by those of skill in the art based on a variety of factors, including the specific host cell, regulatory sequence(s), and vector design in light of the teachings herein.

In one embodiment, a recombinant host cell capable of expressing Tse3 is cultured under conditions suitable for expression of Tse3 from the recombinant vector if no insert is present, and the methods comprise selecting those cells that grow as comprising recombinant vectors with the insert cloned into the expression vector. This method may further comprise plating cells on a first hypo-osmotic media, and on a second hyper-osmotic media, and selecting for recombinant cells on the hypo-osmotic-media. In one embodiment, the hyper-osmotic media contains 1% or greater NaCl, and the hypo-osmotic media contains less than 1% NaCl. The inventors have found that Tse3 is most effective on growing cells under hypo-osmotic conditions, thereby providing a simplified selection protocol.

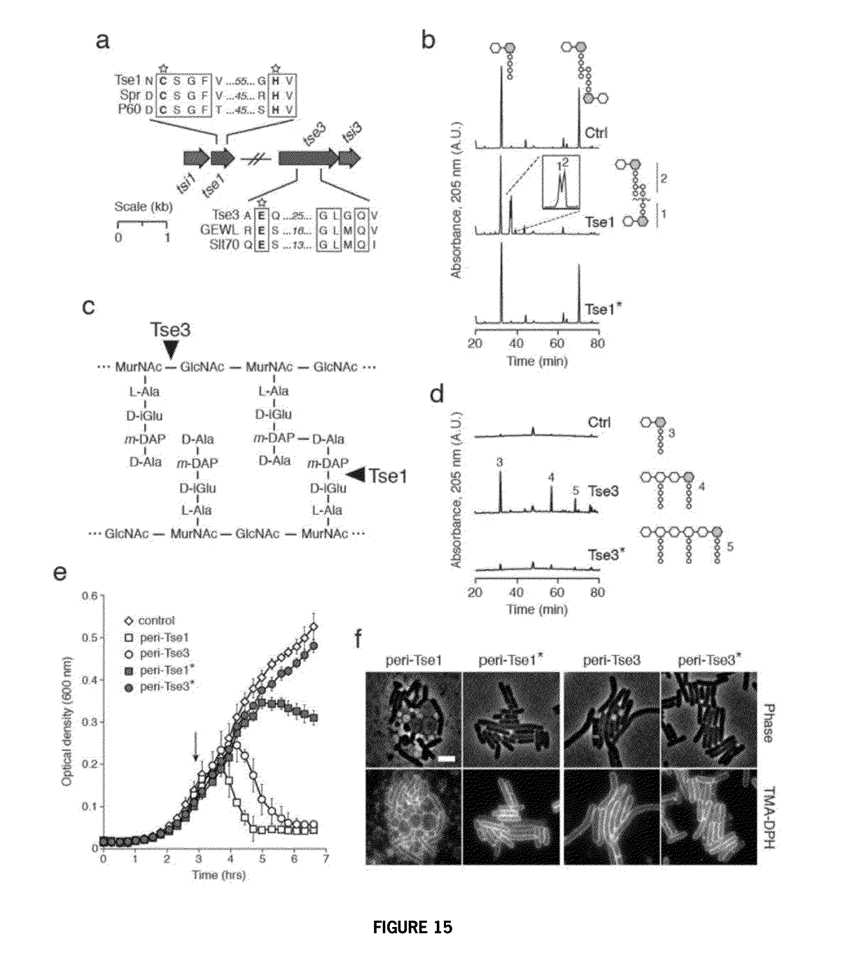

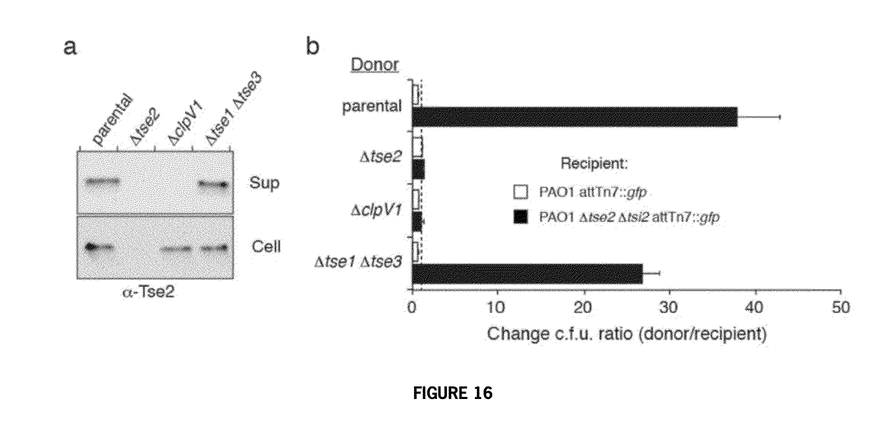

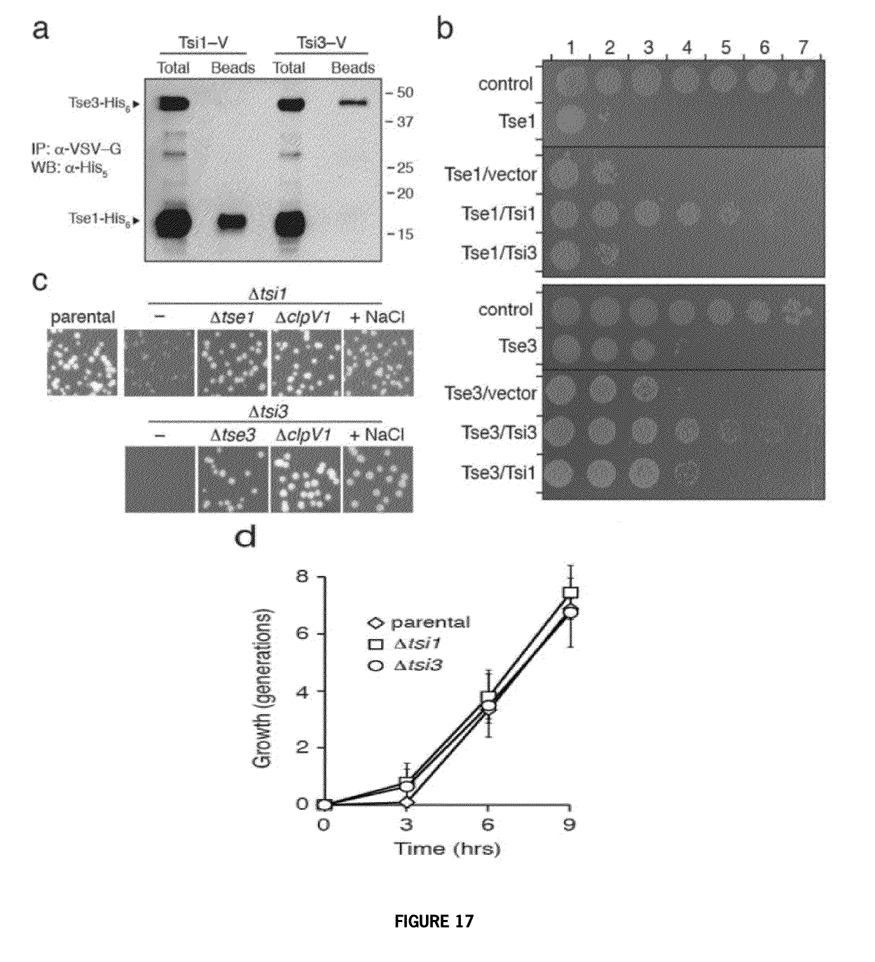

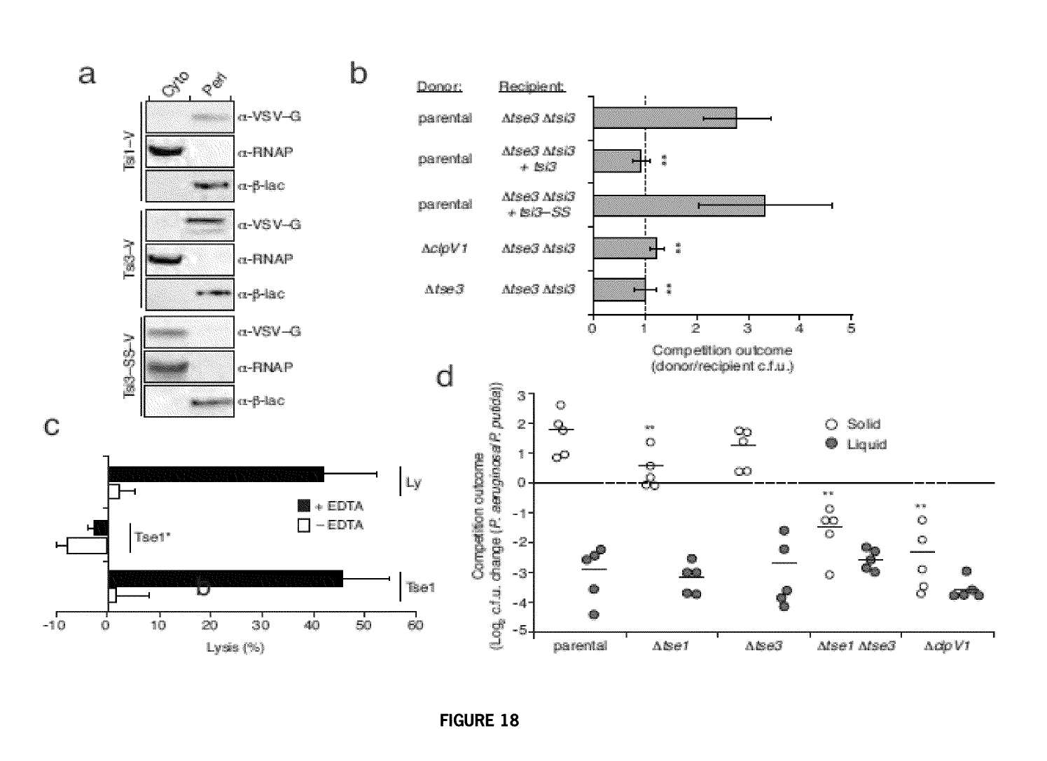

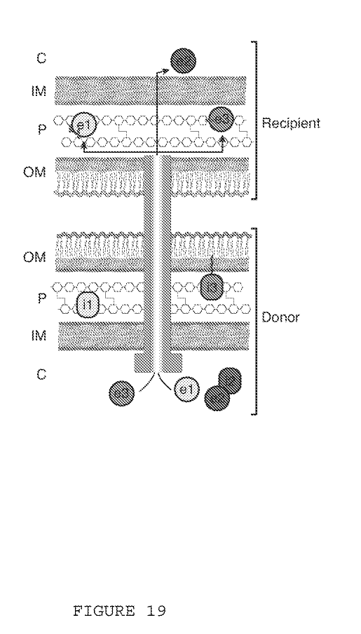

In one embodiment, a recombinant host cell capable of expressing a Tse1 conjugate capable of periplasmic localization (as discussed herein) is cultured under conditions suitable for expression of Tse1 from the recombinant vector if no insert is present, wherein the method comprises culturing the recombinant host cell under conditions suitable for expression of Tse1, and wherein the culture conditions comprise enriching for particular clones by killing off other quickly via Tse1 lytic activity. The inventors have shown that Tse1 possesses bacterial lytic activity, and thus bacterial cells expressing Tse1 are quickly killed. For example, see FIG. 15 and FIG. 18C and the corresponding discussion in the examples that follow. These methods can be used, for example, in selective harvesting of cellular material from cells within a subpopulation, as a substitute for antibiotic use, or to remove unwanted vectors in liquid culture and then harvest from intact cells, saving a day of growth. In one embodiment, the culturing comprises culturing cells under hypo-osmotic conditions, or under non-growing conditions. In another embodiment, the culturing comprises culturing cells in the absence of antibiotic.

In a fourteenth aspect, the present invention provides methods for producing a cloning vector that lacks an insert, comprising culturing the recombinant host cell of any embodiment of the twelfth aspect of the invention under conditions suitable for vector replication and expression of Tse1 or Tse3, wherein the recombinant host cells further express a Tse1 or Tse3 antidote, and isolating vector from the host cells. These methods permit large scale production of the vectors of any embodiment of the present invention. The antidote can be any expression product capable of interfering with the antibacterial activity of Tse1 or Tse3, including but not limited to Tse1 or Tse3 antisense constructs, Tse1 or Tse3-binding aptamers, and Tse1 or Tse3-binding polypeptides. In a preferred embodiment that can be combined with any other embodiment herein, the Tse1 and/or Tse3 antidote comprises any embodiment of Tsi1 and/or Tsi3 disclosed herein. Conditions for cell culture suitable for vector replication can be determined by those of skill in the art based on a variety of factors, including the specific host cell, regulatory sequence(s), and vector design in light of the teachings herein.

In a fifteenth aspect, the invention provides methods for improved biomolecule extraction from bacterial cells, comprising contacting the bacterial cells with an amount effective of Tse1 to lyse the bacterial cells during the extraction process. The inventors have shown that Tse1 possesses potent bacterial lytic activity, thus making it ideal for purification of biomolecules produced in bacteria. In various non-limiting embodiments, the biomolecule comprises of one or more of proteins (including glycoproteins), nucleic acids, polysaccharides, and periplasmic fractions. In these methods, bacterial cells from which biomolecules are to be isolated are treated with an amount effective of Tse1 to lyse the cells. In one non-limiting embodiment, the Tse1 is used at a concentration of between about 1 ng/ml to about 1 mg/ml. In a preferred embodiment, the bacterial cells are non-growing, including but cells removed from culture, concentrated, and resuspended in appropriate buffer for isolating the biomolecules of interest. In one embodiment, the buffer is hypo-osmotic (less than 1% NaCl). In another embodiment, conditions include a Tris-Cl buffer and EDTA for lysis, neither of which should affect downstream applications if desalted. Other suitable conditions for a suitable biomolecule preparation will be readily determined by those of skill in the art, based on the disclosure herein.

In another aspect, the present invention provides host cells comprising in their genome, a first recombinant gene coding for Tse1 and/or Tse3 operatively linked to a regulatory sequence. In one non-limiting embodiment, the recombinant cell comprises a second gene encoding an antidote (such as Tsi1 and/or Tsi3) on a plasmid or a mobile genetic element, and selection for its antidote properties (i.e.: Tse1 and/or Tse3 immunity) maintain that element. In another embodiment, the recombinant host cell comprises a first gene encoding functional Tse1 and/or Tse3 on a plasmid, wherein the recombinant host cell comprises the second gene expressing Tsi1 and/or Tsi3 to permit Tse1 and/or Tse3 plasmid propagation in the host cell. In this embodiment, the second gene can be present on the same or different plasmid, another extra-chromosomal element, or chromosomally integrated. The first gene and second gene are “recombinant” in that the host cell does not endogenously express Tse1 and/or Tse3 or a Tse antidote, and thus Tse1 and/or Tse3 expression requires recombinant expression of Tse1 and/or Tse3, and antidote expression requires recombinant expression of the antidote.

As used herein, “in its genome” includes chromosomal insertion and extra-chromosomal elements, such as plasmids or viral vectors. Thus, in one preferred embodiment, the first recombinant gene and/or the second recombinant gene are present extra-chromosomally. In a further preferred embodiment, the first recombinant gene and/or the second recombinant gene are present as chromosomal insertions. In embodiments in which the second gene coding for an antidote for Tse1 and/or Tse3 is present, the second gene may be on the same, or alternatively, on a different extra-chromosomal element than the first gene, or, alternatively, linked or unlinked to the first gene in the genome. In other embodiments, one of the first and second genes can be a chromosomal insertion, while the other of the first and second genes can be an extra-chromosomal element. In embodiments having the first and second genes are on the same plasmid, the genes can be closely linked. In another embodiment, the first and second genes are on the same plasmid and are not closely linked.

In embodiments where the host cells do not comprise a second recombinant gene coding for an antidote for Tse1 and/or Tse3, the Tse1 and/or Tse3 regulatory sequences are preferably controllable, to control Tse1 and/or Tse3 expression. In exemplary embodiments where the host cells do comprise a second recombinant gene coding for an antidote for Tse1 and/or Tse3, the Tse1 and/or Tse3 regulatory sequence may be inducible and/or the antidote regulatory sequence may be constitutive, to control Tse1 and/or Tse3 expression.

In another non-limiting embodiment, the recombinant cell comprises a first gene encoding Tse1 and/or Tse3 on one plasmid, and a second gene encoding Tsi1 and/or Tsi3 on a second plasmid. In this embodiment, Tsi1 and/or Tsi3 can be used as a selectable marker on an expression vector, wherein the Tse1 and/or Tse3-expressing host cell is introduced into the cells, and only cells expressing Tsi1 and/or Tsi3 will be able to grow. In one embodiment, the regulatory region for Tse1 and/or Tse3 is inducible, and that growth of the cells post-introduction occurs under inducing condition. In this embodiment, the second vector may further comprise a recombinant nucleic acid of interest for expression or other purposes. In embodiments where the Tse1 and/or Tse3 regulatory element is controllable, control of Tse1 and/or Tse3 expression can be used to maintain the Tsi 1 and/or Tsi3 plasmid and/or to select for its integration, providing a way to make stable cells without using an antibiotic.

The present invention also provides kits for carrying out the methods of the invention, and particularly for use in creating the product nucleic acid molecules of the invention or other linked molecules and/or compounds of the invention (e.g., protein-protein, nucleic acid-protein, etc.), or supports comprising such product nucleic acid molecules or linked molecules and/or compounds. The invention also relates to kits for adding and/or removing and/or replacing nucleic acids, proteins and/or other molecules and/or compounds, for creating and using combinatorial libraries of the invention, and for carrying out homologous recombination (particularly gene targeting) according to the methods of the invention.

The kits of the invention may also comprise further components for further manipulating the recombination site-containing molecules and/or compounds produced by the methods of the invention. The kits of the invention may comprise one or more nucleic acid molecules of the invention (particularly starting molecules comprising one or more recombination sites and optionally comprising one or more reactive functional moieties), one or more molecules and/or compounds of the invention, one or more supports of the invention and/or one or more vectors of the invention. Such kits may optionally comprise one or more additional components selected from the group consisting of one or more host cells (e.g., two, three, four, five etc.), one or more reagents for introducing (e.g., by transfection or transformation) molecules or compounds into one or more host cells, one or more nucleotides, one or more polymerases and/or reverse transcriptases (e.g., two, three, four, five, etc.), one or more suitable buffers (e.g., two, three, four, five, etc.), one or more primers (e.g., two, three, four, five, seven, ten, twelve, fifteen, twenty, thirty, fifty, etc.), one or more terminating agents (e.g., two, three, four, five, seven, ten, etc.), one or more populations of molecules for creating combinatorial libraries (e.g., two, three, four, five, seven, ten, twelve, fifteen, twenty, thirty, fifty, etc.) and one or more combinatorial libraries (e.g., two, three, four, five, seven, ten, twelve, fifteen, twenty, thirty, fifty, etc.). The kits of the invention may also contain directions or protocols for carrying out the methods of the invention.

In another aspect the invention provides kits for joining, deleting, or replacing nucleic acid segments, these kits comprising at least one component selected from the group consisting of (1) one or more recombination proteins or compositions comprising one or more recombination proteins, and (2) at least one nucleic acid molecule comprising one or more recombination sites (preferably a vector having at least two different recombination specificities). The kits of the invention may also comprise one or more components selected from the group consisting of (a) additional nucleic acid molecules comprising additional recombination sites; (b) one or more enzymes having ligase activity; (c) one or more enzymes having polymerase activity; (d) one or more enzymes having reverse transcriptase activity; (e) one or more enzymes having restriction endonuclease activity; (f) one or more primers; (g) one or more nucleic acid libraries; (h) one or more supports; (i) one or more buffers; (j) one or more detergents or solutions containing detergents; (k) one or more nucleotides; (l) one or more terminating agents; (m) one or more transfection reagents; (n) one or more host cells; and (o) instructions for using the kit components.

In one embodiment, kits of the invention contain compositions comprising at least one linearized or circular vector containing the Tse1 and/or Tse3; or Tsi1 and/or Tsi3 gene. In some embodiments, the linearized vector contained in the kit is treated such that the ends of the vector are resistant to binding to the other ends of the vector.

In other embodiments, the present invention relates to a kit comprising a carrier or receptacle being compartmentalized to receive and hold therein at least one container, wherein a first container contains linear or circular DNA molecule comprising a vector having at least one DNA fragment of the Tse1 and/or Tse3 gene sequence, as described herein. In another embodiment, the vector contained in the kit has at least one DNA fragment of the Tsi1 and/or Tsi3 gene sequence, as described herein. In another embodiment, the kit contains both vectors which have at least one DNA fragment of the Tse1 and/or Tse3 sequence and vectors that have at least one DNA fragment of the Tsi1 and/or Tsi3 sequence.

All embodiments and combinations of embodiments of Tse1 and/or Tse3 and Tsi1 and/or Tsi3 disclosed above can be used in this aspect of the invention.

The present invention may be better understood with reference to the accompanying examples that are intended for purposes of illustration only and should not be construed to limit the scope of the invention, as defined by the claims appended hereto.

### Example 1

### Summary

The functional spectrum of a secretion system is defined by its substrates. Here we analyzed the secretomes of Pseudomonas aeruginosa mutants altered in regulation of the Hcp Secretion Island-1-encoded type VI secretion system (H1-T655). We identified three substrates of this system, proteins Tse1-3 (type six exported 1-3), which are co-regulated with the secretory apparatus and secreted under tight posttranslational control. The Tse2 protein was found to be the toxin component of a toxin-immunity system, and to arrest the growth of prokaryotic and eukaryotic cells when expressed intracellularly. In contrast, secreted Tse2 had no effect on eukaryotic cells; however, it provided a major growth advantage for P. aeruginosa strains, relative to those lacking immunity, in a manner dependent on cell contact and the H1-T6SS. This demonstration that the T6SS targets a toxin to bacteria helps reconcile the structural and evolutionary relationship between the T6SS and the bacteriophage tail and spike.

### INTRODUCTION

Secreted proteins allow bacteria to intimately interface with their surroundings and other bacteria. The importance and diversity of secreted proteins is reflected in the multitude of pathways bacteria have evolved to enable their export (Abdallah et al., 2007; Filloux, 2009). Large multi-component secretion systems, including types III and IV secretion, have been the focus of a great deal of study because in many organisms they are specialized for effector export and they have the remarkable ability to directly translocate proteins from bacterial to host cell cytoplasm via a needle-like apparatus (Cambronne and Roy, 2006). The recently described type VI secretion system (T6SS) is another specialized system, however its physiological role and general mechanism remain poorly understood (Bingle et al., 2008).

Studies of T6SSs indicate that a functional apparatus requires the products of approximately 15 conserved and closely linked genes, and is strongly correlated to the export of a hexameric ring-shaped protein belonging to the hemolysin co-regulated protein (Hcp) family (Filloux, 2009; Mougous et al., 2006). Hcp proteins are required for assembly of the secretion apparatus and they interact with valine-glycine repeat (Vgr) family proteins, which are also exported by the T6SS. The function of the Hcp/Vgr complex remains unclear, however it is believed that the proteins are extracellular structural components of the secretion apparatus. Recent X-ray crystallographic insights into Hcp and Vgr-family proteins show that they are similar to bacteriophage tube and tail spike proteins, respectively (Leiman et al., 2009; Pell et al., 2009). These findings prompted speculation that the T6SS is evolutionarily, structurally, and mechanistically related to bacteriophage. According to this model, the T6SS assembles as an inverted phage tail on the surface of the bacterium, with the Hcp/Vgr complex forming the distal end of the cell-puncturing device. Another notable conserved T6S gene product is ClpV, a AAA+-family ATPase that has been postulated to provide the energy necessary to drive the secretory apparatus (Mougous et al., 2006). The roles of the remaining conserved T6S proteins remain largely unknown.

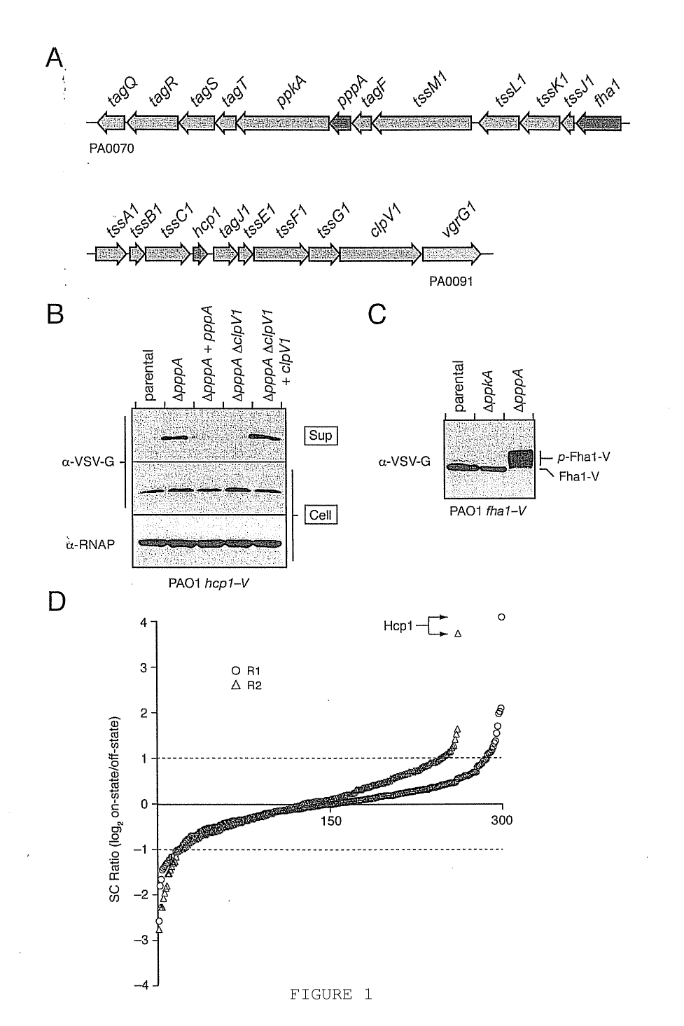

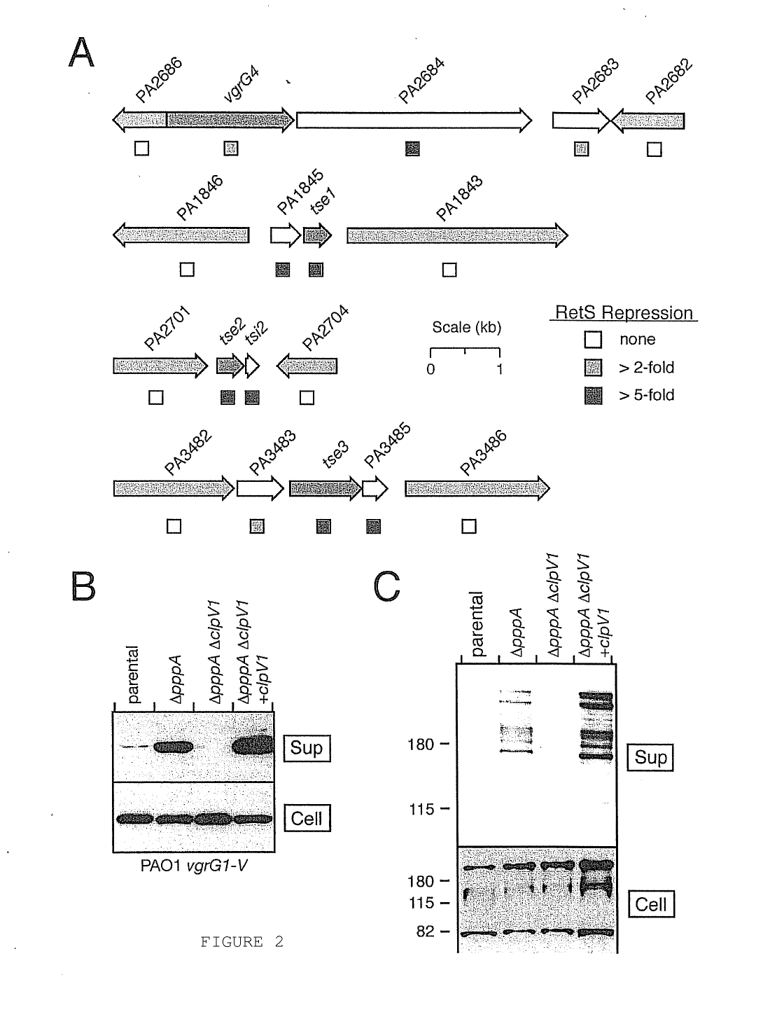

Nonconserved genes encoding predicted accessory elements are also linked to most T6SSs (Bingle et al., 2008). In the HSI-1-encoded T6SS of Pseudomonas aeruginosa (H1-T6SS) (FIG. 1A), these genes encode elements of a posttranslational regulatory pathway that strictly modulates the activity of the secretion system through changes in the phosphorylation state of a forkhead-associated domain protein, Fha1 (Mougous et al., 2007). Phosphorylation of Fha1 by a transmembrane serine-threonine Hanks-type kinase, PpkA, triggers Hcp1 secretion. PppA, a PP2C-type phosphatase, antagonizes Fha1 phosphorylation.

The T6SS has been linked to a myriad of processes, including biofilm formation (Aschtgen et al., 2008; Enos-Berlage et al., 2005), conjugation (Das et al., 2002), quorum sensing regulation (Weber et al., 2009), and both promoting and limiting virulence (Filloux, 2009). The P. aeruginosa H1-T6SS has been implicated in the fitness of the bacterium in a chronic infection; mutants in conserved genes in this secretion system failed to efficiently replicate in a rat lung chronic infection model and the system was shown to be active in cystic fibrosis (CF) patient infections (Mougous et al., 2006; Potvin et al., 2003). The H1-T6SS is also co-regulated with other chronic infection virulence factors such as the psl and pel loci, which are involved in biofilm formation (Goodman et al., 2004; Ryder et al., 2007).

How the apparently conserved T6SS architecture can participate in such a wide range of activities is not clear. At least one mechanism by which the secretion system can exert its effects on a host cell has been garnered from studies of Vibrio cholerae. A T6S-associated VgrG-family protein of this organism contains a domain with actin-crosslinking activity that is translocated into host cell cytoplasm in a process requiring endocytosis and cell-cell contact (Ma et al., 2009; Pukatzki et al., 2007; Satchell, 2009). The subset of VgrG-family proteins that contain non-structural domains with conceivable roles in pathogenesis have been termed “evolved” VgrG proteins (Pukatzki et al., 2007). This configuration, wherein an effector domain is presumably translocated into host cell cytoplasm by virtue of its fusion to the T6S cell puncturing apparatus, is intriguing, but it is likely not general; a multitude of organisms containing T6SSs do not encode “evolved” VgrG proteins (Boyer et al., 2009; Pukatzki et al., 2009).

Key to understanding the function of the T6SS—as with any secretion system—is to identify and characterize the protein substrates that it exports. EvpP from Edwardsiella tarda and RbsB from Rhizobium leguminosarum are proposed substrates of the system; however, inconsistent with anticipated properties of T6S substrates, RbsB contains an N-terminal Sec secretion signal, and EvpP stably associates with a component of the secretion apparatus (Bladergroen et al., 2003; Pukatzki et al., 2009; Zheng and Leung, 2007).

In this study, we identified three proteins, termed Tse1-3 (type VI secretion exported 1-3), that are substrates of the H1-T6SS of P. aeruginosa. We showed that one of these, Tse2, is the toxin component of a toxin-immunity system, and that it is able to arrest the growth of a variety of prokaryotic and eukaryotic organisms. Despite the promiscuity of toxin expressed intracellularly, we found that H1-T655-exported Tse2 was specifically targeted to bacteria. In growth competition experiments, immunity to Tse2 provided a marked growth advantage in a manner dependent on intimate cell-cell contact and a functional H1-T6SS. The ability of the secretion system to efficiently target Tse2 to a bacterium, and not to a eukaryotic cell, suggests that T6S may play a role in the delivery of toxin and effector molecules between bacteria.

### Results

### Design and Characterization of H1-T6SS On- and Off-State Strains

Under laboratory culturing conditions, activation of the H1-T6SS is strongly repressed at the posttranslational level by the phosphatase PppA (FIG. 1A). We have shown that inactivation of pppA leads to Hcp1 export, and that this could reflect triggering of the “on-state” in the secretory apparatus (Hsu et al., 2009; Mougous et al., 2007). These observations led us to predict that additional components of the apparatus, and even substrates of the secretion system, are also exported in this state. To identify these proteins, we sought to compare the secretomes of ΔpppΔ and AclpV1. The latter lacks the H1-T6SS ATPase, ClpV1, and therefore remains in the “off-state” (FIG. 1A) (Mougous et al., 2006).

To probe whether the on-state and off-state mutations could modulate the activity of the H1-T6SS, we assayed their effect on Hcp1 secretion in P. aeruginosa PAO1 hcp1-V (where present, -V denotes a fusion of the indicated gene to a sequence encoding the vesicular stomatitis virus G epitope). As expected, the deletion of pppA promoted Hcp1 secretion and Fha1 phosphorylation relative to the parental strain (FIGS. 1B and C). Since the wild-type strain does not secrete Hcp1 to detectable levels, the effects of ΔclpV1 were gauged using the ΔpppA background. Introduction of the clpV1 deletion to ΔpppA abrogated Hcp1 secretion and this effect was fully complemented by ectopic expression of clpV1 (FIG. 1B). These data indicate that pppA and clpV1 deletions are sufficient to activate and inactivate the H1-T6SS secretion system, respectively.

### Mass Spectrometric Analysis of on- and Off-State Secretomes

Next, we used MS and spectral counting to compare proteins present in the secretomes of the on- and off-state P. aeruginosa strains (Liu et al., 2004). Average spectral count (SC) values were used to identify whether each protein was differentially secreted between states. The results of our MS analyses are summarized in Table S1. Importantly, the total number of spectral counts was comparable between the on- and off-states in both replicates. A total of 371 proteins that met our filtering criteria were identified between replicate experiments (Tables S2). We divided the proteins into three groups: Category 1 (C1; Tables S3 and S4)—present in both the on- and off-states, Category 2 (C2; Table S5)—present only in the on-state, and Category 3 (C3; Table S6)—present only in the off-state. Overlap between the replicates was greatest among C1 proteins. A total of 314 C1 proteins were identified, of which 249 were shared between the replicates. A significant fraction of the C1 differences can be ascribed to the fact that 13% more proteins were identified in this category in Replicate 1 (R1) than in Replicate 2 (R2).

To assess the accuracy of the quantitative component of our datasets, we measured the distribution of SC ratios (on-state/off-state) within Cl proteins (FIG. 1D). Since we did not anticipate that the H1-T6SS should exhibit a global effect on the secretome, we were encouraged by the approximate split (50%±2 in both replicates) between those proteins that were up-versus down-regulated between the on- and off-states. Additionally, the change in average SCs between the states was low, and this value was similar in the replicates ([R1], 1.13±1.04; [R2], 1.15±0.90). Only 30 R1 and 33 R2 proteins yielded a SC ratio>2.

As expected, Hcp1 was over-represented in the on-state samples. Indeed, Hcp1 was the most differentially secreted protein in both datasets (SC ratio: [R1], 13; [R2], 17]) (FIG. 1D). The presence of Hcp1 in the secretome of off-state cells suggests a certain extent of cellular protein contamination within the preparations. This contamination is also evidenced by the predicted or known functions of many of the detected proteins (Tables S2-S4). The high abundance of Hcp1 (119 SC average) relative to the average protein abundance (10.9 SC) is likely another factor contributing to its detection in the off-state samples.

Next we analyzed C2 proteins—those observed only in the on-state. Similar numbers of these proteins were identified in R1 (19) and R2 (20), and five of these were found in both replicates (Table 1). The reproducibility of C2 versus C1 proteins is attributable to the difference in their average SCs; the average SC of C2 proteins was 2.6, versus 12 in C1. The C2 proteins identified in both R1 and R2 accounted for five of the six most abundant in C2-R1, and five of the ten most abundant in C2-R2. Each of these proteins lacked a secretion signal for known export pathways. The identity of these proteins and the biochemical validation of their secretion is the subject of subsequent sections.

The number and abundance of C3 proteins in both R1 and R2 was slightly lower than the corresponding C2 values. Nonetheless, we did identify three C3 proteins in common between R1 and R2 (Table 1). The occurrence of these proteins in the off-state is likely to reflect changes in gene regulation caused by modulation of the activity of the H1-T6SS that manifest in the secretome. Sequence analysis indicated that each of these proteins contains a predicted signal peptide (Emanuelsson et al., 2007).

### Two VgrG Proteins are Secreted by the H1-T6SS

Two VgrG-family proteins, the products of open reading frames PA0091 and PA2685, were the most abundant C2 proteins in R1 and R2 (Table 1). Interestingly, earlier microarray work has shown that PA0091 and PA2685 are coordinately regulated with HSI-I by the RetS hybrid two-component sensor/response regulator protein, however the participation of these proteins in the H1-T6SS was not investigated (FIG. 2A) (Goodman et al., 2004; Laskowski and Kazmierczak, 2006; Zolfaghar et al., 2005). The PA0091 locus is located within HSI-I, while the PA2685 locus is found at an unlinked site that lacks other apparent T6S elements (FIGS. 1A and 2A). To remain consistent with previous nomenclature, these genes will henceforth be referred to as vgrG1 and vgrG4 (Mougous et al., 2006).

To confirm the MS results, we compared the localization of VgrG1 and VgrG4 in wild-type bacteria to strains containing the on-state (ΔpppA) and off-state (ΔclpV1) mutations. Consistent with our MS findings, Western blot analyses of cell and supernatant fractions in vgrG1-V and vgrG4-V backgrounds indicated that secretion of the proteins is strongly repressed by pppA and requires clpV1 (FIGS. 2B and 2C). These data show that the H1-T6SS exports at least two VgrG-family proteins. For reasons not yet understood, VgrG4-V migrated as two major bands in the cellular fraction and a large number of high molecular weight bands in the supernatant.

### Identification of Three H1-T6SS Substrates

The remaining C2 proteins identified in both R1 and R2 are proteins encoded by ORFs PA1844, PA2702, and PA3484. Interestingly, an earlier study identified the product of PA1844 as an immunogenic protein expressed by a P. aeruginosa clinical isolate (Wehmhoner et al., 2003). Bioinformatic analyses of the three proteins indicated that they do not share detectable sequence homology to each other or to proteins outside of P. aeruginosa. Each protein is encoded by an ORF that resides in a predicted two-gene operon with a second hypothetical ORF. Intriguingly, we noted that the three unlinked operons—like HSI-I (which includes vgrG1) and vgrG4—are negatively regulated by RetS (FIG. 2A).

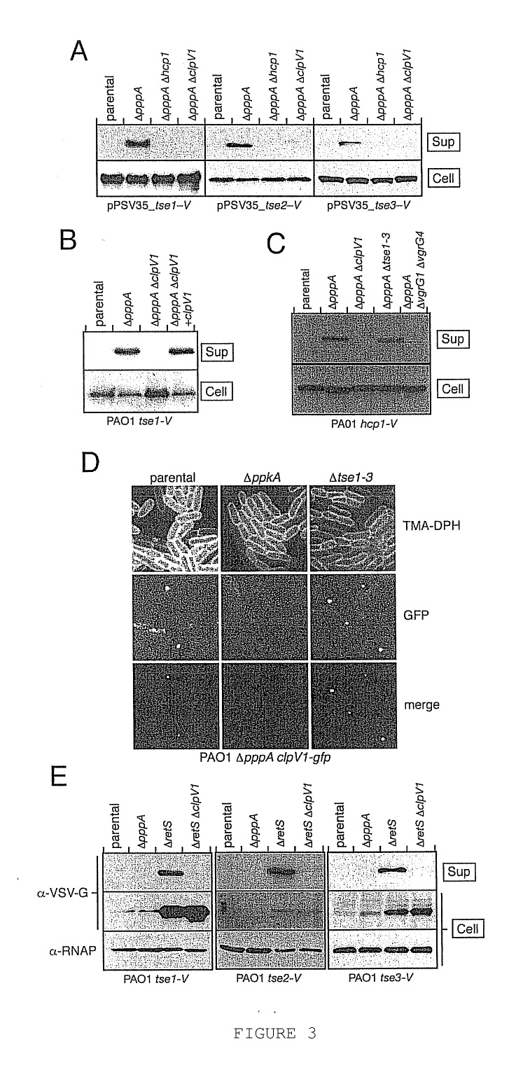

Based on our secretome analyses, we hypothesized that the proteins encoded by PA1844, PA2702, and PA3484, henceforth referred to Tse1-3, respectively, are substrates of the H1-T6SS. To test this, we analyzed the localization of the proteins when ectopically expressed in a diagnostic panel of P. aeruginosa strains. The secretion profile of each protein was similar in these strains; relative to the wild-type, ΔpppA displayed dramatically increased levels of secretion, and secretion levels were at or below wild-type levels in ΔpppA strains containing additional deletions in either hcp1 or clpV1 (FIG. 3A). Over-expression of the proteins was ruled out as a confounding factor, as the secretion profile of chromosomally-encoded Tse1-V in related backgrounds was similar to that of the ectopically-expressed protein (FIG. 3B). Finally, we complemented Tse1-V secretion in ΔpppA ΔclpV1 tse1-V with a plasmid expressing clpV1.

To further distinguish the Tse proteins as H1-T6SS substrates rather than structural components, we determined their influence on core functions of the T6 secretion apparatus. Fundamental to each studied T6SS is the ability to secrete an Hcp-related protein. In a systematic analysis, Hcp secretion was shown to require all predicted core T6SS components, including VgrG-family proteins (Pukatzki et al., 2007; Zheng and Leung, 2007). We generated a strain containing a deletion of all tse genes in the ΔpppA hcp1-V background and compared Hcp1 secretion in this strain to strains lacking both vgrG1 and vgrG4 or clpV1 in the same background. Western blot analysis revealed that Hcp1 secretion was abolished in both the ΔclpV1 and AvgrG1 ΔvgrG4 strains, however it was unaffected by tse deletion (FIG. 3C).

A multiprotein complex containing ClpV1 is essential for a functional T6S apparatus (Hsu et al., 2009). As a second indicator of H1-T6SS function, we used fluorescence microscopy to examine the formation of this complex in strains containing a chromosomal fusion of clpV1 to a sequence encoding the green fluorescent protein (clpV1-GFP) (Mougous et al., 2006). In line with the Hcp1 secretion result, the punctate appearance of ClpV1-GFP localization, which is indicative of proper apparatus assembly, was not dependent on the tse genes (FIG. 3D). On the other hand, deletion of ppkA, a gene required for assembly of the H1-T6S apparatus, disrupted ClpV1-GFP localization. Together, these findings provide evidence that the Tse proteins are substrates of H1-T6SS.

### Tse Secretion is Triggered by De-Repression of the Gac/Rsm Pathway

Earlier microarray experiments suggested that the tse genes are tightly repressed by RetS, a component of the Gac/Rsm signaling pathway (Lapouge et al., 2008). In this pathway, the activity of RetS and two other sensor kinase enzymes, LadS and GacS, converge to reciprocally regulate an overlapping group of acute and chronic virulence pathways in P. aeruginosa through the small RNA-binding protein RsmA (Brencic and Lory, 2009; Goodman et al., 2004; Ventre et al., 2006). To directly investigate the effect of the Gac/Rsm pathway on tse expression, we monitored the abundance of Tse proteins in the cell-associated and secreted fractions of strains containing the rets deletion. Our data showed that activation of the Gac/Rsm pathway dramatically elevates cellular Tse levels and triggers their export via the H1-T6SS (FIG. 3E). It is noteworthy that secretion of Tse proteins in ΔretS is far in excess of that observed in ΔpppA (FIG. 3E, compare ΔpppA and ΔretS).

Tsi2 is an Essential Protein that Protects P. aeruginosa from Tse2

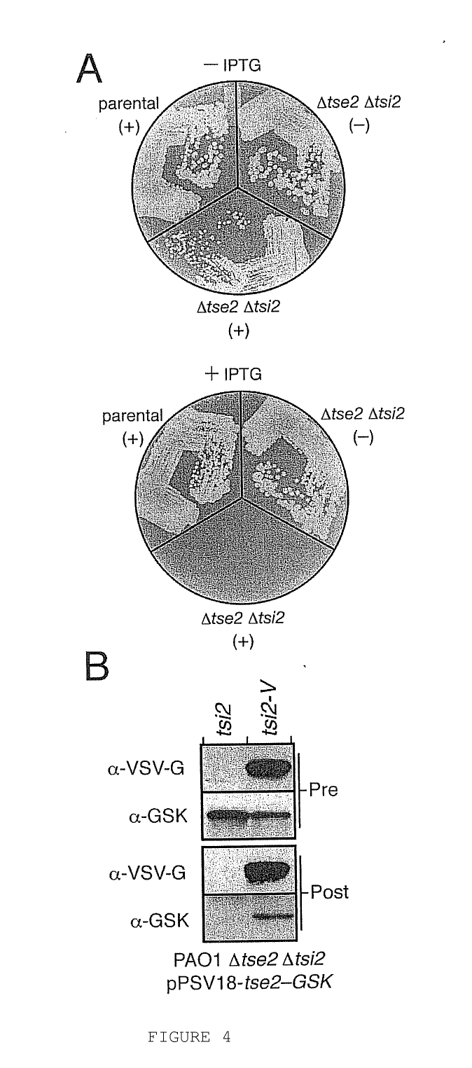

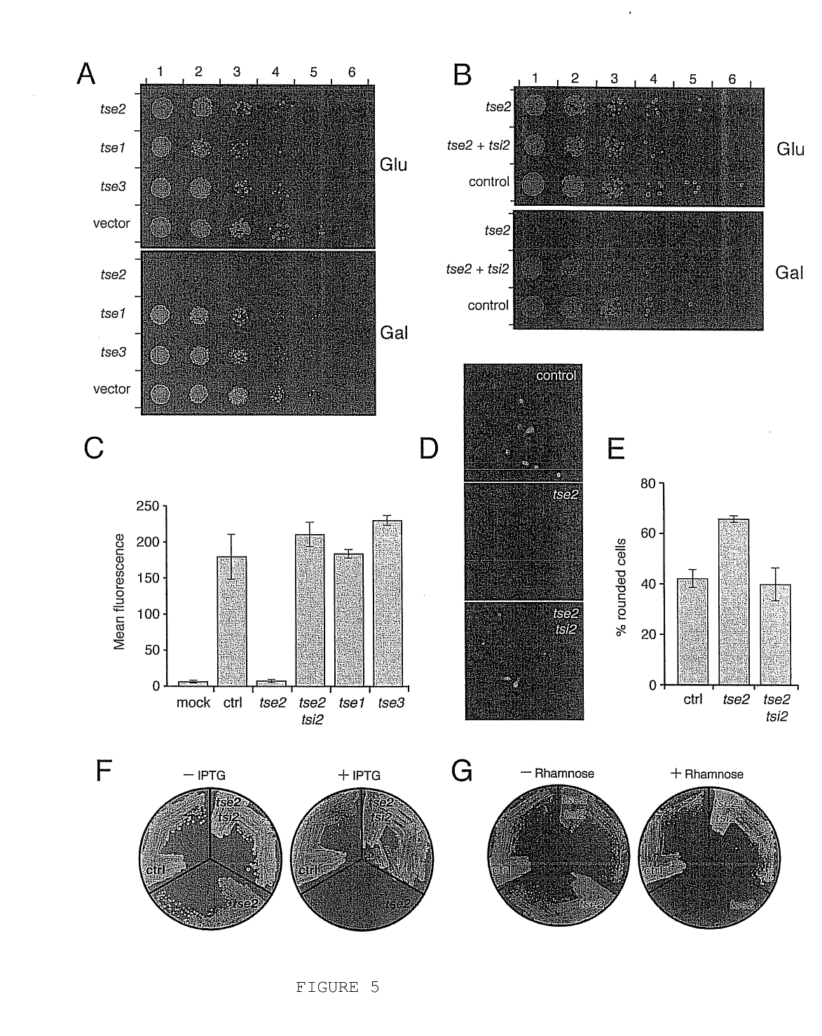

The lack of transposon insertions within the tse2/tsi2 locus in a published transposon insertion library of P. aeruginosa PAO1 suggested that these ORFs may be essential for viability of the organism (Jacobs et al., 2003). To test this possibility, we attempted to generate deletions of tse2 and tsi2. While a Δtse2 strain was readily constructed, tsi2 was refractory to several methods of deletion. Based on genetic context and co-regulation (FIG. 2A), we hypothesized that Tse2 and Tsi2 could interact functionally, and that the requirement for tsi2 could therefore depend on tse2. Success in simultaneous deletion of both genes confirmed this hypothesis (FIG. 4A).

Our findings implied that Tsi2 protects cells from Tse2. To probe this possibility further, we introduced tse2 to the Δtse2 Δtsi2 background. Induction of tse2 expression completely abrogated growth of Δtse2 Δtsi2, however it had only a mild effect on wild-type cells. These data demonstrate that tse2 encodes a toxic protein capable of inhibiting the growth of P. aeruginosa, and that tsi2 encodes a cognate immunity protein. We named Tsi2 based on this property (type VI secretion immunity protein 2).

Tsi2 could block the activity of Tse2 through a mechanism involving direct interaction of the proteins, or by an indirect mechanism wherein the proteins function antagonistically on a common pathway. To determine if Tse2 and Tsi2 physically interact, we conducted co-immunoprecipitation studies in P. aeruginosa. Tse2 was specifically identified in precipitate of Tsi2-V, indicative of a stable Tse2-Tsi2 complex (FIG. 4B). These data provide additional support for a functional interaction between Tse2 and Tsi2, and they suggest that the mechanism of Tsi2 inhibition of Tse2 is likely to involve physical association of the proteins.

### Intracellular Tse2 is Toxic to a Broad Spectrum of Prokaryotic and Eukaryotic Cells

P. aeruginosa is widely dispersed in terrestrial and aquatic environments, and it is also an opportunistic pathogen with a diverse host range. As such, Tse2 exported from P. aeruginosa has the potential to interact with a range of organisms, including prokaryotes and eukaryotes. To investigate the organisms that Tse2 might target, we expressed tse2 in the cytoplasm of representative species from each domain. Two eukaryotic cells were chosen for our investigation, Saccharomyces cerevisiae and the HeLa human epithelial-derived cell line. Yeast were included primarily for diversity, however these organisms also interact with P. aeruginosa in assorted environments and could therefore represent a target of the toxin (Wargo and Hogan, 2006). S. cerevisiae cells were transformed with a galactose-inducible expression plasmid for each tse gene, or with an empty control plasmid (Mumberg et al., 1995). Relative to the other tse genes and the control, tse2 expression caused a dramatic decrease in observable colony forming units following 48 hrs of growth under inducing conditions (FIG. 5A). To address the specificity of Tse2 effects on S. cerevisiae, we next tested whether Tsi2 could block Tse2-mediated toxicity. Co-expresssion of tsi2 with tse2 restored viability to levels similar to the control strain (FIG. 5B). This result implies that the effects of Tse2 on S. cerevisiae are specific and that the toxin may act via a similar mechanism in bacteria and yeast. Our findings are consistent with an earlier screen for P. aeruginosa proteins toxic to yeast. Arnoldo et al. found Tse2 among nine P. aeruginosa proteins most toxic to S. cerevisiae within a library of 505 that included known virulence factors (Arnoldo et al., 2008).

The effects of Tse2 on a mammalian cell were probed using a reporter co-transfection assay in HeLa cells. Expression plasmids containing the tse genes were generated and mixed with a GFP reporter plasmid. Co-transfection of the reporter plasmid with tse1 and tse3 had no impact on GFP expression relative to the control; however, inclusion of the tse2 plasmid reduced GFP expression to background levels (FIGS. 5C and 5D). We also noted morphological differences between cells transfected with tse2 and control transfections, which was apparent in the fraction of rounded cells (FIG. 5E). These were specific effects of Tse2, as the inclusion of a tsi2 expression plasmid into the tse2/GFP reporter plasmid transfection restored GFP expression and lowered the fraction of rounded cells to the control. From these studies, we conclude that Tse2 has a deleterious effect on essential cellular processes in assorted eukaryotic cell types.

Next we asked whether Tse2 has activity in prokaryotes other than P. aeruginosa. We tested two organisms, Escherichia coli and Burkholderia thailandensis. Both organisms were transformed with plasmids engineered for inducible expression of either tse2, or as a control, both tse2 and tsi2. In each case, tse2 expression strongly inhibited growth and co-expression with tsi2 reversed this effect (FIGS. 5F and 5G). Taken together with the effects we observed in S. cerevisiae and HeLa cells, we conclude that Tse2 is a toxin that—when administered intracellularly—inhibits essential cellular processes in a broad spectrum of organisms.

P. aeruginosa can Target Bacterial, but not Eukaryotic Cells, with Tse2

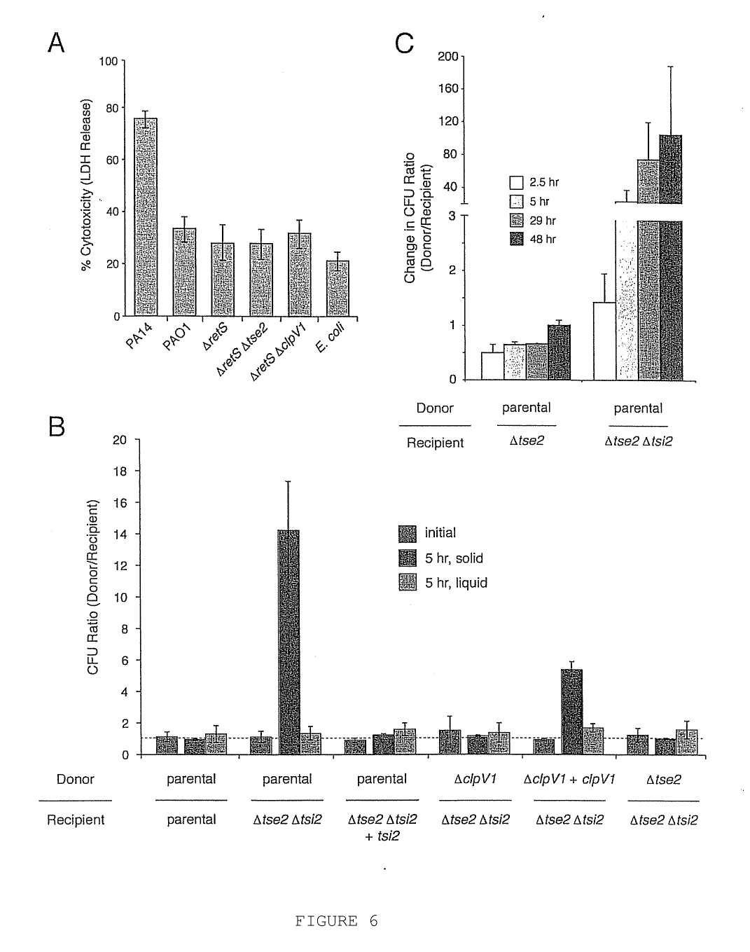

Since tse2 expression experiments indicated that the toxin could act on eukaryotes (FIG. 5A-E), we asked whether P. aeruginosa could target these cells with the H1-T6SS. We measured cytotoxicity toward HeLa and J774 cells for a panel of P. aeruginosa strains, including Tse2 hyper-secreting (ΔretS) and non-secreting backgrounds (ΔretS ΔclpV1). Under all conditions analyzed, we were unable to observe Tse2-promoted cytotoxicity or a morphological impact on the cells as was observed in transfection experiments (FIG. 6A and data not shown). Additionally, attempts to detect Tse2 or other Tse proteins in mammalian cell cytoplasm yielded no evidence of translocation (data not shown). We also investigated Tse2-dependent effects on yeast co-cultured with P. aeruginosa; again, no effect could be attributed to Tse2 (Figure S1). Based on our data, we concluded that P. aeruginosa is unlikely to utilize Tse2 as a toxin against eukaryotic cells. This is in-line with results of earlier reports, which have shown that strains lacking rets are highly attenuated in acute virulence-related phenotypes, including macrophage and epithelial cell cytotoxicity (Goodman et al., 2004; Zolfaghar et al., 2005), and acute pneumonia and corneal infections in mice (Zolfaghar et al., 2006) (Laskowski et al., 2004).

The influence of intracellular tse2 expression on the growth of bacteria prompted us to next investigate whether its target could be another prokaryotic cell. To test this, we conducted a series of in vitro growth competition experiments with P. aeruginosa strains in the ΔretS background engineered with regard to their ability to produce, secrete, or resist Tse2. Competitions between these strains were conducted in liquid medium or following filtration onto a porous solid support. Neither production nor secretion of Tse2, nor immunity to the toxin, impacted the growth rates of competing strains in liquid medium (FIG. 6B). On the contrary, a striking proliferative advantage dependent on tse2 and tsi2 was observed when cells were grown on a solid support. In growth competition experiments between ΔretS and ΔretS Δtse2 Δtsi2, henceforth referred to as donor and recipients strains, respectively, donor cells were approximately 14-fold more abundant after 5 hours (FIG. 6B). This was entirely Tse2 mediated, as a deletion of tse2 from the donor strain, or the addition of tsi2 to the recipient strain, abrogated the growth advantage. Inactivation of clpV1 within the donor strain confirmed that the Tse2-mediated growth advantage requires a functional H1-T6SS (FIG. 6B). Importantly, the total proliferation of the donor remained constant in each experiment, indicating that Tse2 suppresses growth of the recipient strain.

In order to examine the extent to which Tse2 could facilitate a growth advantage, we conducted long-term competitions between strains with and without Tse2 immunity. The experiments were initiated with a donor-to-recipient cell ratio of approximately 10:1, raising the probability that each recipient cell will contact a donor cell. After 48 hours, the Tse2 donor strain displayed a remarkable 104-fold growth advantage relative to a recipient strain lacking immunity (FIG. 6C). These data conclusively demonstrate that the P. aeruginosa H1-T6SS can target Tse2 to another bacterial cell. The differences observed between competitions conducted in liquid medium versus on a solid support suggest that intimate donor-recipient cell contact is required. We have not directly demonstrated that Tse2 is translocated into recipient cell cytoplasm, however it is a likely explanation for our data given that cell contact is required and Tsi2 is a cytoplasmic immunity protein that physically interacts with the toxin (FIG. 4B).

### Discussion

The T6SS has been implicated in numerous, apparently disparate processes. With few exceptions, the mode-of-action of the secretion system in these processes is not known. Since the T6SS architecture appears highly conserved, we based our study on the supposition that the diverse activities of T6SSs, including T6SSs within a single organism, must be attributable to a diverse array of substrate proteins exported in a specific manner by each system. Our findings support this model; we identified three T6S substrates that lack orthologs outside of P. aeruginosa, and that specifically require the H1-T6SS for their export (FIGS. 1 and 3).

Bacterial genomes encode a large and diverse array of toxin-immunity protein (TI) systems (Gerdes et al., 2005). These can be important for plasmid maintenance, stress response, programmed cell death, cell-fate commitment, and defense against other bacteria. Tse2 differs from other TI toxins in that it is exported through a large, specialized secretion apparatus, while many TI system toxins are either not actively secreted, or they utilize the sec pathway (Riley and Wertz, 2002). This distinction implies that secretion through the T6S apparatus is required to target Tse2 to a relevant environment, cell, or subcellular compartment. Indeed, we have shown that targeting of Tse2 by the T6S apparatus is essential for its activity (FIG. 6).

We found that Tse2 is active against assorted bacteria and eukaryotic cells when expressed intracellularly (FIGS. 4 and 5). Despite this, we found no evidence that P. aeruginosa can target Tse2 to a eukaryotic cell, including mammalian cells of epithelial and macrophage origin (FIG. 6A and data not shown). Surprisingly, P. aeruginosa efficiently targeted the toxin to another bacterial cell (FIG. 6). These findings, combined with the following recent observations, provide support for the hypothesis that the T6SS can serve as an inter-bacterial interaction pathway. First, the secretion system is present and conserved in many non-pathogenic, solitary bacteria (Bingle et al., 2008; Boyer et al., 2009). Second, there is experimental evidence supporting an evolutionary relationship between extracellular components of the secretion apparatus and the tail proteins of bacteriophages T4 and X (Ballister et al., 2008; Leiman et al., 2009; Pell et al., 2009; Pukatzki et al., 2007). Finally, two recent reports have implicated the conserved T6S component, VgrG, in inter-bacterial interactions. A bioinformatic analysis of Salmonella genomes identified a group of “evolved” VgrG proteins bearing C-terminal effector domains highly related to bacteria-targeting S-type pyocins, and a VgrG protein from Proteus mirabilis was shown to participate in an intra-species self/non-self recognition pathway (Blondel et al., 2009; Gibbs et al., 2008).

It is also evident that in certain instances the T6SS has evolved to engage eukaryotic cells. In at least two reports, the T6S apparatus has been demonstrated to deliver a protein to a eukaryotic cell (Ma et al., 2009; Suarez et al., 2009). Moreover, the T6SSs of several pathogenic bacteria are major virulence factors (Bingle et al., 2008). Taken together with our findings, we posit that there are two broad groups of T6SSs, those that target bacteria and those that target eukaryotes. It is not possible at this time to rule out that a given T6SS may have dual specificity. However, our inability to detect the effects of Tse2 in an infection of a eukaryotic cell, and the fact that a Tse2 hyper-secreting strain is attenuated in animal models of acute infection (Laskowski et al., 2004; Zolfaghar et al., 2006), suggests that the T6S apparatus can be highly discriminatory. In this regard, it is instructive to consider other secretion systems that have evolved from inter-bacterial interaction pathways. The type IVA and type IVB secretion systems are postulated to have evolved from a bacterial conjugation system ((Burns, 2003; Christie et al., 2005; Lawley et al., 2003). These systems have become efficient at eukaryotic cell intoxication, however measurements indicate that substrate translocation into bacteria occurs at a frequency of only ˜1×10⁻⁶/donor cell (Luo and Isberg, 2004). In contrast, Tse2 targeting to bacteria by the H1-T6SS appears many orders of magnitude more efficient, as the donor strain in our assays is able to effectively suppress the net growth of an equal amount of recipient cells. The host adapted type IV secretion systems and the H1-T6SS represent two apparent extremes in the cellular targeting specificity of Gram-negative specialized secretion systems. Furthermore, they show that a high degree of discrimination can exist between pathways targeting eukaryotes and prokaryotes.

The physiologically relevant target bacteria of Tse2 and the H1-T6SS remains an open question. We have initiated studies to address the role of these factors in interspecies interactions, however we have not yet identified an effect. This may be because diffusible anti-bacterial molecules released by P. aeruginosa dominate the outcome of growth competitions performed under the conditions used in FIG. 6 (Hoffman et al., 2006; Kessler et al., 1993; Voggu et al., 2006). In future studies designed to allow free diffusion of these factors, and thereby more closely mimic a natural setting, their role may be mitigated. Interestingly, all sequenced P. aeruginosa strains appear to encode orthologs of tse2 and tsi2. Additionally, we found the genes universally present within a library of 44 randomly selected CF patient clinical isolates (Figure S2). Despite these findings, it remains possible that Tse2-mediated inter-P. aeruginosa interactions could be relevant in a natural context. For instance, it may not be simply the presence or absence of the toxin or its immunity protein, but rather the extent and manner in which these traits are expressed that decides the outcome of an interaction. In prior investigations of clinical isolates, we noted a high degree of heterogeneity in H1-T6SS activation, as judged by Hcp1 secretion levels (Mougous et al., 2006; Mougous et al., 2007). The wild-type strain used in the current study does not secrete Hcp1, and in this background the H1-T6SS does not provide a growth advantage against an immunity-deficient strain (data not shown). However, the H1-T6SS activation state of many clinical isolates resembles the ΔretS background, and therefore these strains are likely capable of using Tse2 in competition with other bacteria. In this context, it is intriguing that tse and HSI-I expression are subject to strict regulation by the Gac/Rsm pathway (FIG. 3E). Since this pathway responds to bacterial signals, including those of the sensing strain and other Pseudomonads (Lapouge et al., 2008), it is conceivable that cell-cell recognition could be an important aspect of Tse2 production and resistance.

The cell-cell contact requirement of H1-T6SS-dependent delivery of Tse2 suggest that the system could play an important role in scenarios involving relatively immobile cells, such as cells encased in a biofilm. The polyclonal and polymicrobial lung infections of patients with CF, wherein the bacteria are thought to reside within biofilm-like structures, is one setting where Tse2 could provide a fitness advantage to P. aeruginosa (Sibley et al., 2006; Singh et al., 2000). Intriguingly, P. aeruginosa is particularly adept at adapting to and competing in this environment, and studies have shown that it can even displace preexisting bacteria (D'Argenio et al., 2007; Deretic et al., 1995; Hoffman et al., 2006; Nguyen and Singh, 2006) (Foundation, 2007). If Tse2 does play a key role in the fitness of P. aeruginosa in a CF infection, this could explain the elevated expression and activation of the H1-T6SS observed in isolates from CF patients (Mougous et al., 2006; Mougous et al., 2007; Starkey et al., 2009; Yahr, 2006).

### Experimental Procedures

### Bacterial Strains, Plasmids and Growth Conditions

The P. aeruginosa strains used in this study were derived from the sequenced strain PAO1 (Stover et al., 2000). P. aeruginosa were grown on Luria-Bertani (LB) medium at 37° C. supplemented with 30 μg ml⁻¹ gentamicin, 300 μg ml⁻¹ carbenicillin, 25 μg ml⁻¹ irgasan, 5% w/v sucrose, 0.5 mM IPTG and 40 μg ml⁻¹ X-gal (5-bromo-4-chloro-3-indolyl(3-D-galactopyranoside) as required. Burkholderia thailandensis E264 and Escherichia coli BL21 were grown on LB medium containing 200 μg ml⁻¹ trimethoprim, 50 μg ml⁻¹ kanamycin, 0.2% w/v glucose, 0.2% w/v rhamnose and 0.5 mM IPTG as required. E. coli SM10 used for conjugation with P. aeruginosa was grown in LB medium containing 15 μg ml⁻¹ gentamicin. Plasmids used for inducible expression include pPSV35, pPSV35CV, and pSW196 for P. aeruginosa (Baynham et al., 2006; Hsu et al., 2009; Rietsch et al., 2005), pET29b (Novagen) for E. coli, pSCrhaB2 (Cardona and Valvano, 2005) for B. thailandensis, and p426-GAL-L and p423-GAL-L for S. cerevisiae (Mumberg et al., 1995). Chromosomal fusions and gene deletions were generated as described previously (Mougous et al., 2006; Rietsch et al., 2005). See Supplemental Experimental Procedures for specific cloning procedures.

### Secretome Preparation

Cells were grown to optical density 600 nm (OD₆₀₀) 1.0 in Vogel-Bonner minimal medium containing 19 mM amino acids as defined in synthetic CF sputum medium (Palmer et al., 2007). The presence of amino acids was required for H1-T6SS activity (data not shown). Proteins were prepared as described previously (Wehmhoner et al., 2003).

### Mass Spectrometry

Precipitated proteins were suspended in 100 μl of 6 M urea in 50 mM NH₄HCO₃, reduced and alkylated with dithiotreitol and iodoactamide, respectively, and digested with trypsin (50:1 protein:trypsin ratio). The resultant peptides were desalted with Vydac C18 columns (The Nest Group) following the manufacturer's protocol. Samples were dried to 5 μL, resuspended in 0.1% formic acid/5% acetonitrile and analyzed on an LTQ-Orbitrap mass spectrometer (Thermo Fisher) in triplicate. Data was searched using Sequest (Eng et al., 1994) and validated with Peptide/Protein Prophet (Keller et al., 2002). The relative abundance for identified proteins was calculated using spectral counting (Liu et al., 2004). See Supplemental Experimental Procedures additional MS procedures.

### Preparation of Proteins and Western Blotting

Cell-associated and supernatant samples were prepared as described previously (Hsu et al., 2009). Western blotting was performed as described previously (Mougous et al., 2006), with the exception that detection of the Tse proteins required primary antibody incubation in 5% BSA in Tris-buffered saline containing 0.05% v/v Tween 20 (TBST). The GSK tag was detected using α-GSK (Cell Signaling Technologies).

### Immunoprecipitation

Cells grown in appropriate additives were harvested at mid-log phase by centrifugation (6,000×g, 3 min) at 4° C. and resuspended in 10 ml of Buffer 1 (200 mM NaCl, 20 mM Tris pH 7.5, 5% glycerol, 2 mM dithiothreitol, 0.1% triton) containing protease inhibitors (Sigma) and lysozyme (0.2 mg ml⁻¹). Cells were disrupted by sonication and the resulting lysate was clarified by centrifugation (25,000×g, 30 min) at 4° C. A sample of the supernatant material was removed (Pre) and the remainder was incubated with 100 μl of α-VSV-G agarose beads (Sigma) for 2 hours at 4° C. for. Beads were washed three times with 15 ml of Buffer 1 and pelleted by centrifugation. Proteins were eluted with SDS-PAGE loading buffer.

### Fluorescence Microscopy

Mid-log phase cultures were harvested by centrifugation (6,000×g, 3 min), washed with phosphate-buffered saline (PBS), and resuspended to OD₆₀₀ 5 with PBS containing 0.5 mM TMA-DPH (Molecular Probes). Microscopy was performed as described previously (Hsu et al., 2009). All images shown were manipulated identically.

### Yeast Toxicity Assays

Saccharomyces cerevisiae BY4742 (MATα his3Δl leu2Δ0 lys2Δ0 ura3Δ0) was transformed with p426-GAL-L containing tse1, tse2, tse3, or the empty vector, and grown o/n in SC−Ura+2% glucose (Mumberg et al., 1995). Cultures were resuspended to OD₆₀₀ 1.0 with water and serially diluted fivefold onto SC−Ura+2% glucose agar or SC−Ura+2% galactose+2% raffinose agar. Plates were incubated at 30° C. for 2 days before being photographed. The tsi2 gene was cloned into p423-GAL-L and transformed into S. cerevisiae BY4742 harboring the p426-GAL-L plasmid. Cultures were grown o/n in SC−Ura−His+2% glucose.

### Growth Competition Assays

Overnight cultures were mixed at the appropriate donor-to-recipient ratio to a total density of approximately 1.0×10⁸ CFU/ml in 5 ml LB medium. In each experiment, either the donor or recipient strain contained lacZ inserted at the neutral phage attachment site (Vance et al., 2005). This gene had no effect on competition outcome. Co-cultures were either filtered onto a 47-mm 0.2 μm nitrocellulose membrane (Nalgene) and placed onto LB agar or were inoculated 1:100 into 2 ml LB (containing 0.4% w/v L-arabinose, if required), and were incubated at 37° C. with shaking Filter-grown cells were resuspended in LB medium and plated on LB agar containing X-gal.

### Cell Culture and Infection Assays

HeLa cells were cultured and maintained in Dulbecco's modified eagle medium (DMEM, Invitrogen) supplemented with 10% Fetal Bovine Serum (FBS) and 100 μml⁻¹ penicillin or streptomycin as required. Incubations were performed at 37° C. in the presence of 5% CO₂. Infection assays were carried out using cells seeded in 96-well plates at a density of 2.0×10⁴ cells/well. Following o/n incubation, wells were washed in 1× Hank's balanced salt solution and DMEM lacking sodium pyruvate and antibiotics was added. Bacterial inoculum was added to wells at a multiplicity of infection of 50 from cultures of OD₆₀₀ 1.0. Following incubation for 5 hours, the percent cytotoxicity was measured using the CytoTox-One assay (Promega).

### Transient Transfection, Cell Rounding Assays, and Flow Cytometric Analysis

HeLa cells were seeded in 24-well flat bottom plates at a density of 2.0×10⁵ cells/well and incubated o/n in DMEM supplemented with 10% FBS. Reporter co-transfection experiments were performed using Lipofectamine according to the manufacturer's protocol. Total amounts of transfected DNA were normalized using equal quantities of the GFP reporter plasmid (empty pEGFP-N1 (Clonetech)), one of the tse expression plasmids (pEGFP-N-1-derived), and either a non-specific plasmid or the tsi2 expression plasmid where indicated. Cell rounding was quantified manually using phase-contrast images from three random fields acquired at 40× magnification. Prior to flow cytometry, HeLa cells were washed two times and resuspended in 1×PBS supplemented with 0.75% FBS. Analysis was performed on a BD FACscan2 cell analyzer and mean GFP intensities were calculated using FlowJo 7.5 software (Tree Star, Inc.).

### Example 2

Tse2 and Tsi2 mutants were generated and tested for cytotoxic activity and preservation of immunity to Tse2 cytotoxicity, respectively.

Truncation mutants listed in Table 1 were tested for (a) toxicity as judged by ectopic expression of allele in P. aeruginosa PAO1 Δtse2 Δtsi2., (b) expression as determined by α-VSV-G Western blot, and (c) secretion determined by presence of indicated protein in concentrated supernatants prepared from PAO1 ΔretS Δtse2 versus PAO1 ΔretS Δtse2 ΔclpV1. The mutants listed in Table 1 are based on the P. aeruginosa PAO1 sequence (SEQ ID NO:2). All truncations were fused at their C-terminus to the VSV-G epitope.

TABLE 1
Toxicity and secretion via T6S of Tse2 truncation mutants.

| Tse2 residues | | | |
|---|---|---|---|
| present¹ | Toxicity² | Expression³ | Secretion⁴ |
| 7-158 | + | + | + |
| 10-158 | − | +− | − |
| 13-158 | − | +− | − |
| 16-158 | − | +− | − |
| 19-158 | − | +− | − |
| 22-158 | − | +− | − |
| 32-158 | − | + | − |
| 44-158 | − | + | − |
| 1-120 | − | + | − |
| 1-125 | − | + | − |
| 1-129 | − | + | − |
| 1-155 | + | + | + |

The lack of toxicity observed for those alleles that did not express fully (+/−) could be attributed to expression levels. The data presented in Table 1 show that Tse2 residues 1-6 and 156-158 are not required for toxicity.

A variety of Tse2 point mutants (Table 2) were also generated by Quikchange mutagenesis in the pPSV35-CV vector (see Hsu and Mougous, 2009 for plasmid reference). Toxicity, expression, and secretion were assessed as for the truncation mutants in Table 1.

TABLE 2
Toxicity and secretion via T6S of Tse2 point mutants.

| Tse2 amino acid | | | |
|---|---|---|---|
| substitution(s) | Toxicity | Expression | Secretion |
| S9A L10A | + | + | N/D |
| R60A | +− | + | + |
| T79A S80A | − | + | + |
| R89AR90A | − | N/D | N/D |
| Q119A | + | + | + |
| KP129-130AA | +− | + | − |
| QL139-140AA | + | + | N/D |
| RR149-150AA | + | + | N/D |

We next generated a series of Tsi2 mutants and tested for Tse2-immunity properties. Immunity was determined by ectopic expression of the indicated allele in P. aeruginosa Δtse2 Δtsi2. Growth of the strain indicates Tsi2 provides immunity, as Tse2 is co-expressed. Numbering of the Ts12 sequence is relative to the Tsi2 sequence of SEQ ID NO:4.

TABLE 3
Tsi2 mutants and associated Tse2-immunity properties.

| Mutants | Immunity | Mutants | Immunity |
|---|---|---|---|
| pET29-Tse2-Tsi2-cv (wild-type) | + | pET29-Tse2-Tsi2-D30A-cv | + |
| pET29-Tse2-Tsi2-alpha-cv | − | pET29-Tse2-Tsi2-Q32A-cv | + |
| pET29-Tse2-Tsi2-N2A-cv | + | pET29-Tse2-Tsi2-N33A-cv | + |
| pET29-Tse2-Tsi2-K4A-cv | + | pET29-Tse2-Tsi2-E36Acv | + |
| pET29-Tse2-Tsi2-Q6A-cv | + | pET29-Tse2-Tsi2-E38A-cv | + |
| pET29-Tse2-Tsi2-T7A-cv | + | pET29-Tse2-Tsi2-Q39A-cv | + |
| pET29-Tse2-Tsi2-L8A-cv | + | pET29-Tse2-Tsi2-Y44A-cv | + |
| pET29-Tse2-Tsi2-Q13A-cv | + | pET29-Tse2-Tsi2-D45A-cv | + |
| pET29-Tse2-Tsi2-R18A-cv | + | pET29-Tse2-Tsi2-D49A-cv | + |
| pET29-Tse2-Tsi2-R20A-cv | + | pET29-Tse2-Tsi2-D50A-cv | + |
| pET29-Tse2-Tsi2-E21A-cv | + | pET29-Tse2-Tsi2-K52A-cv | + |
| pET29-Tse2-Tsi2-Q25A-cv | + | pET29-Tse2-Tsi2-E56A-cv | + |
| pET29-Tse2-Tsi2-Q27A-cv | + | pET29-Tse2-Tsi2-Q57A-cv | + |
| pET29-Tse2-Tsi2-N28A-cv | + | pET29-Tse2-Tsi2-Q61A-cv | + |
| pET29-Tse2-Tsi2-D29A-cv | + | pET29-Tse2-Tsi2-A47Q-cv | +− |
| pET29-Tse2-Tsi2-V10Q-cv | + | pET29-Tse2-Tsi2-A11Q-cv | +− |
| pET29-Tse2-Tsi2-C14Q-cv | + | pET29-Tse2-Tsi2-V42Q-cv | + |
| pET29-Tse2-Tsi2-L46Q-cv | + | pET29-Tse2-Tsi2-1-59-cv | + |

The data presented in Table 3 show that mutations at virtually all positions retained tse2 immunity, demonstrating that Tsi2 is resilient and its interactions are robust. Truncation studies (not shown) demonstrated that residues 60-77 of Tsi2 can be removed while retaining its Tse2 immunity activity.

### REFERENCES FOR EXAMPLES 1-2

Abdallah, A. M., Gey van Pittius, N. C., Champion, P. A., Cox, J., Luirink, J., Vandenbroucke-Grauls, C. M., Appelmelk, B. J., and Bitter, W. (2007). Type VII secretion—mycobacteria show the way. Nat Rev Microbiol 5, 883-891.
Arnoldo, A., Curak, J., Kittanakom, S., Chevelev, I., Lee, V. T., Sahebol-Amri, M., Koscik, B., Ljuma, L., Roy, P. J., Bedalov, A., et al. (2008). Identification of small molecule inhibitors of Pseudomonas aeruginosa exoenzyme S using a yeast phenotypic screen. PLoS genetics 4, e1000005.
Aschtgen, M. S., Bernard, C. S., De Bentzmann, S., Lloubes, R., and Cascales, E. (2008). SciN is an outer membrane lipoprotein required for Type VI secretion in enteroaggregative Escherichia coli. J. Bacteriol.
Ballister, E. R., Lai, A. H., Zuckermann, R. N., Cheng, Y., and Mougous, J. D. (2008). In Vitro Self-Assembly of Tailorable Nanotubes from a Simple Protein Building Block. Proc Natl Acad Sci USA 105, 3733-3738.
Baynham, P. J., Ramsey, D. M., Gvozdyev, B. V., Cordonnier, E. M., and Wozniak, D. J. (2006). The Pseudomonas aeruginosa ribbon-helix-helix DNA-binding protein AlgZ (AmrZ) controls twitching motility and biogenesis of type IV pili. J Bacteriol 188, 132-140.
Bingle, L. E., Bailey, C. M., and Pallen, M. J. (2008). Type VI secretion: a beginner's guide. Current opinion in microbiology 11, 3-8.
Bladergroen, M. R., Badelt, K., and Spaink, H. P. (2003). Infection-blocking genes of a symbiotic Rhizobium leguminosarum strain that are involved in temperature-dependent protein secretion. Mol Plant Microbe Interact 16, 53-64.
Blondel, C. J., Jimenez, J. C., Contreras, I., and Santiviago, C. A. (2009). Comparative genomic analysis uncovers 3 novel loci encoding type six secretion systems differentially distributed in Salmonella serotypes. BMC genomics 10, 354.
Boyer, F., Fichant, G., Berthod, J., Vandenbrouck, Y., and Attree, I. (2009). Dissecting the bacterial type VI secretion system by a genome wide in silico analysis: what can be learned from available microbial genomic resources? BMC genomics 10, 104.
Brencic, A., and Lory, S. (2009). Determination of the regulon and identification of novel mRNA targets of Pseudomonas aeruginosa RsmA. Mol Microbiol 72, 612-632.
Burns, D. L. (2003). Type IV transporters of pathogenic bacteria. Current opinion in microbiology 6, 29-34.
Cambronne, E. D., and Roy, C. R. (2006). Recognition and delivery of effector proteins into eukaryotic cells by bacterial secretion systems. Traffic (Copenhagen, Denmark) 7, 929-939.
Cardona, S. T., and Valvano, M. A. (2005). An expression vector containing a rhamnose-inducible promoter provides tightly regulated gene expression in Burkholderia cenocepacia. Plasmid 54, 219-228.
Christie, P. J., Atmakuri, K., Krishnamoorthy, V., Jakubowski, S., and Cascales, E. (2005). Biogenesis, architecture, and function of bacterial type iv secretion systems. Annual review of microbiology 59, 451-485.
D'Argenio, D. A., Wu, M., Hoffman, L. R., Kulasekara, H. D., Deziel, E., Smith, E. E., Nguyen, H., Ernst, R. K., Larson Freeman, T. J., Spencer, D. H., et al. (2007). Growth phenotypes of Pseudomonas aeruginosa lasR mutants adapted to the airways of cystic fibrosis patients. Mol Microbiol 64, 512-533.
Das, S., Chakrabortty, A., Banerjee, R., and Chaudhuri, K. (2002). Involvement of in vivo induced icmF gene of Vibrio cholerae in motility, adherence to epithelial cells, and conjugation frequency. Biochem Biophys Res Commun 295, 922-928.
Deretic, V., Schurr, M. J., and Yu, H. (1995). Pseudomonas aeruginosa, mucoidy and the chronic infection phenotype in cystic fibrosis. Trends Microbiol 3, 351-356.
Emanuelsson, O., Brunak, S., von Heijne, G., and Nielsen, H. (2007). Locating proteins in the cell using TargetP, SignalP and related tools. Nature protocols 2, 953-971.
Eng, J. K., McCormack, A. L., and Yates, J. R. (1994). An approach to correlate tandem mass-spectral data of peptides with amino-acid sequences in a protein database. J Am Soc Mass Spectrom 5, 976-989.
Enos-Berlage, J. L., Guvener, Z. T., Keenan, C. E., and McCarter, L. L. (2005). Genetic determinants of biofilm development of opaque and translucent Vibrio parahaemolyticus. Mol Microbiol 55, 1160-1182.
Filloux, A. (2009). The type VI secretion system: a tubular story. The EMBO journal 28, 309-310.
Foundation, C. F. (2007). Cystic Fibrosis Foundation Patient Registry, (Cystic Fibrosis Found, Bethesda, Md.)
Garcia, J. T., Ferracci, F., Jackson, M. W., Joseph, S. S., Pattis, I., Plano, L. R., Fischer, W., and Plano, G. V. (2006). Measurement of effector protein injection by type III and type IV secretion systems by using a 13-residue phosphorylatable glycogen synthase kinase tag. Infection and immunity 74, 5645-5657.
Gerdes, K., Christensen, S. K., and Lobner-Olesen, A. (2005). Prokaryotic toxin-antitoxin stress response loci. Nat Rev Microbiol 3, 371-382.
Gibbs, K. A., Urbanowski, M. L., and Greenberg, E. P. (2008). Genetic determinants of self identity and social recognition in bacteria. Science 321, 256-259.
Goodman, A. L., Kulasekara, B., Rietsch, A., Boyd, D., Smith, R. S., and Lory, S. (2004). A signaling network reciprocally regulates genes associated with acute infection and chronic persistence in Pseudomonas aeruginosa. Dev Cell 7, 745-754.
Hoffman, L. R., Deziel, E., D'Argenio, D. A., Lepine, F., Emerson, J., McNamara, S., Gibson, R. L., Ramsey, B. W., and Miller, S. I. (2006). Selection for Staphylococcus aureus small-colony variants due to growth in the presence of Pseudomonas aeruginosa. Proc Natl Acad Sci USA 103, 19890-19895.
Hsu, F., Schwarz, S., and Mougous, J. D. (2009). TagR promotes PpkA-catalysed type VI secretion activation in Pseudomonas aeruginosa. Mol Microbiol 72, 1111-1125.
Jacobs, M. A., Alwood, A., Thaipisuttikul, I., Spencer, D., Haugen, E., Ernst, S., Will, O., Kaul, R., Raymond, C., Levy, R., et al. (2003). Comprehensive transposon mutant library of Pseudomonas aeruginosa. Proc Natl Acad Sci USA 100, 14339-14344.
Keller, A., Nesvizhskii, A. I., Kolker, E., and Aebersold, R. (2002). Empirical statistical model to estimate the accuracy of peptide identifications made by MS/MS and database search. Analytical chemistry 74, 5383-5392.
Kessler, E., Safrin, M., Olson, J. C., and Ohman, D. E. (1993). Secreted LasA of Pseudomonas aeruginosa is a staphylolytic protease. The Journal of biological chemistry 268, 7503-7508.
Lapouge, K., Schubert, M., Allain, F. H., and Haas, D. (2008). Gac/Rsm signal transduction pathway of gamma-proteobacteria: from RNA recognition to regulation of social behaviour. Mol Microbiol 67, 241-253.
Laskowski, M. A., and Kazmierczak, B. I. (2006). Mutational analysis of RetS, an unusual sensor kinase-response regulator hybrid required for Pseudomonas aeruginosa virulence. Infection and immunity 74, 4462-4473.
Laskowski, M. A., Osborn, E., and Kazmierczak, B. I. (2004). A novel sensor kinase-response regulator hybrid regulates type III secretion and is required for virulence in Pseudomonas aeruginosa. Mol Microbiol 54, 1090-1103.
Lawley, T. D., Klimke, W. A., Gubbins, M. J., and Frost, L. S. (2003). F factor conjugation is a true type IV secretion system. FEMS Microbiol Lett 224, 1-15.
Leiman, P. G., Basler, M., Ramagopal, U. A., Bonanno, J. B., Sauder, J. M., Pukatzki, S., Burley, S. K., Almo, S. C., and Mekalanos, J. J. (2009). Type VI secretion apparatus and phage tail-associated protein complexes share a common evolutionary origin. Proc Natl Acad Sci USA 106, 4154-4159.
Liu, H., Sadygov, R. G., and Yates, J. R., 3rd (2004). A model for random sampling and estimation of relative protein abundance in shotgun proteomics. Analytical chemistry 76, 4193-4201.
Luo, Z. Q., and Isberg, R. R. (2004). Multiple substrates of the Legionella pneumophila Dot/Icm system identified by interbacterial protein transfer. Proc Natl Acad Sci USA 101, 841-846.
Ma, A. T., McAuley, S., Pukatzki, S., and Mekalanos, J. J. (2009). Translocation of a Vibrio cholerae type VI secretion effector requires bacterial endocytosis by host cells. Cell host & microbe 5, 234-243.
Mougous, J. D., Cuff, M. E., Raunser, S., Shen, A., Zhou, M., Gifford, C. A., Goodman, A. L., Joachimiak, G., Ordonez, C. L., Lory, S., et al. (2006). A virulence locus of Pseudomonas aeruginosa encodes a protein secretion apparatus. Science 312, 1526-1530.
Mougous, J. D., Gifford, C. A., Ramsdell, T. L., and Mekalanos, J. J. (2007). Threonine phosphorylation post-translationally regulates protein secretion in Pseudomonas aeruginosa. Nature cell biology 9, 797-803.
Mumberg, D., Muller, R., and Funk, M. (1995). Yeast vectors for the controlled expression of heterologous proteins in different genetic backgrounds. Gene 156, 119-122.
Nguyen, D., and Singh, P. K. (2006). Evolving stealth: genetic adaptation of Pseudomonas aeruginosa during cystic fibrosis infections. Proc Natl Acad Sci USA 103, 8305-8306.
Palmer, K. L., Aye, L. M., and Whiteley, M. (2007). Nutritional cues control Pseudomonas aeruginosa multicellular behavior in cystic fibrosis sputum. J Bacteriol 189, 8079-8087.
Pell, L. G., Kanelis, V., Donaldson, L. W., Howell, P. L., and Davidson, A. R. (2009). The phage lambda major tail protein structure reveals a common evolution for long-tailed phages and the type VI bacterial secretion system. Proc Natl Acad Sci USA 106, 4160-4165.
Potvin, E., Lehoux, D. E., Kukavica-Ibrulj, I., Richard, K. L., Sanschagrin, F., Lau, G. W., and Levesque, R. C. (2003). In vivo functional genomics of Pseudomonas aeruginosa for high-throughput screening of new virulence factors and antibacterial targets. Environmental microbiology 5, 1294-1308.
Pukatzki, S., Ma, A. T., Revel, A. T., Sturtevant, D., and Mekalanos, J. J. (2007). Type VI secretion system translocates a phage tail spike-like protein into target cells where it cross-links actin. Proc Natl Acad Sci USA 104, 15508-15513.
Pukatzki, S., McAuley, S. B., and Miyata, S. T. (2009). The type VI secretion system: translocation of effectors and effector-domains. Current opinion in microbiology 12,11-17.
Rietsch, A., Vallet-Gely, I., Dove, S. L., and Mekalanos, J. J. (2005). ExsE, a secreted regulator of type III secretion genes in Pseudomonas aeruginosa. Proc Natl Acad Sci U S A 102, 8006-8011.
Riley, M. A., and Wertz, J. E. (2002). Bacteriocins: evolution, ecology, and application. Annual review of microbiology 56,117-137.
Ryder, C., Byrd, M., and Wozniak, D. J. (2007). Role of polysaccharides in Pseudomonas aeruginosa biofilm development. Current opinion in microbiology 10, 644-648.
Satchell, K. J. (2009). Bacterial martyrdom: phagocytes disabled by type VI secretion after engulfing bacteria. Cell host & microbe 5, 213-214.
Sibley, C. D., Rabin, H., and Surette, M. G. (2006). Cystic fibrosis: a polymicrobial infectious disease. Future microbiology 1, 53-61.
Singh, P. K., Schaefer, A. L., Parsek, M. R., Moninger, T. O., Welsh, M. J., and Greenberg, E. P. (2000). Quorum-sensing signals indicate that cystic fibrosis lungs are infected with bacterial biofilms. Nature 407, 762-764.
Starkey, M., Hickman, J. H., Ma, L., Zhang, N., De Long, S., Hinz, A., Palacios, S., Manoil, C., Kirisits, M. J., Starner, T. D., et al. (2009). Pseudomonas aeruginosa rugose small-colony variants have adaptations that likely promote persistence in the cystic fibrosis lung. J Bacteriol 191, 3492-3503.
Stover, C. K., Pham, X. Q., Erwin, A. L., Mizoguchi, S. D., Warrener, P., Hickey, M. J., Brinkman, F. S., Huihagle, W. O., Kowalik, D. J., Lagrou, M., et al. (2000). Complete genome sequence of Pseudomonas aeruginosa PA01, an opportunistic pathogen. Nature 406, 959-964.
Suarez, G., Sierra, J. C., Erova, T. E., Sha, J., Horneman, A. J., and Chopra, A. K. (2009). A type VI secretion system effector protein VgrG1 from Aeromonas hydrophila that induces host cell toxicity by ADP-ribosylation of actin. J. Bacteriol.
Vance, R. E., Rietsch, A., and Mekalanos, J. J. (2005). Role of the type III secreted exoenzymes S, T, and Y in systemic spread of Pseudomonas aeruginosa PAO1 in vivo. Infection and immunity 73, 1706-1713.
Ventre, I., Goodman, A. L., Vallet-Gely, I., Vasseur, P., Soscia, C., Molin, S., Bleves, S., Lazdunski, A., Lory, S., and Filloux, A. (2006). Multiple sensors control reciprocal expression of Pseudomonas aeruginosa regulatory RNA and virulence genes. Proc Natl Acad Sci USA 103, 171-176.
Voggu, L., Schlag, S., Biswas, R., Rosenstein, R., Rausch, C., and Gotz, F. (2006). Microevolution of cytochrome bd oxidase in Staphylococci and its implication in resistance to respiratory toxins released by Pseudomonas. J Bacteriol 188, 8079-8086.
Wargo, M. J., and Hogan, D. A. (2006). Fungal—bacterial interactions: a mixed bag of mingling microbes. Current opinion in microbiology 9, 359-364.
Weber, B., Hasic, M., Chen, C., Wai, S. N., and Milton, D. L. (2009). Type VI secretion modulates quorum sensing and stress response in Vibrio anguillarum. Environmental microbiology.
Wehmhoner, D., Haussler, S., Tummler, B., Jansch, L., Bredenbruch, F., Wehland, J., and Steinmetz, I. (2003). Inter- and intraclonal diversity of the Pseudomonas aeruginosa proteome manifests within the secretome. J Bacteriol 185, 5807-5814.
Yahr, T. L. (2006). A critical new pathway for toxin secretion? N Engl J Med 355, 1171-1172.
Zheng, J., and Leung, K. Y. (2007). Dissection of a type VI secretion system in Edwardsiella tarda. Mol Microbiol 66, 1192-1206.
Zolfaghar, I., Angus, A. A., Kang, P. J., To, A., Evans, D. J., and Fleiszig, S. M. (2005). Mutation of retS, encoding a putative hybrid two-component regulatory protein in Pseudomonas aeruginosa, attenuates multiple virulence mechanisms. Microbes Infect 7, 1305-1316.
Zolfaghar, I., Evans, D. J., Ronaghi, R., and Fleiszig, S. M. (2006). Type III secretion-dependent modulation of innate immunity as one of multiple factors regulated by Pseudomonas aeruginosa RetS. Infection and immunity 74, 3880-3889.

### Example 3

### Burkholderia Type VI Secretion Systems have Distinct Roles in Eukaryotic and Bacterial Cell Interactions

### ABSTRACT

Bacteria that live in the environment have evolved pathways specialized to defend against eukaryotic organisms or other bacteria. In this manuscript, we systematically examined the role of the five type VI secretion systems (T6SSs) of Burkholderia thailandensis (B. thai) in eukaryotic and bacterial cell interactions. Consistent with phylogenetic analyses comparing the distribution of the B. thai T6SSs with well characterized bacterial and eukaryotic cell-targeting T6SSs, we found that T6SS-5 plays a critical role in the virulence of the organism in a murine melioidosis model, while a strain lacking the other four T6SSs remained as virulent as the wild-type. The function of T6SS-5 appeared to be specialized to the host and not related to an in vivo growth defect, as ΔT6SS-5 was fully virulent in mice lacking MyD88. Next we probed the role of the five systems in interbacterial interactions. From a group of 31 diverse bacteria, we identified several organisms that competed less effectively against wild-type B. thai than a strain lacking T6SS-1 function. Inactivation of T6SS-1 renders B. thai greatly more susceptible to cell contact-induced stasis by Pseudomonas putida, Pseudomonas fluorescens and Serratia proteamaculans—leaving it 100- to 1000-fold less fit than the wild-type in competition experiments with these organisms. Flow cell biofilm assays showed that T6S-dependent interbacterial interactions are likely relevant in the environment. B. thai cells lacking T6SS-1 were rapidly displaced in mixed biofilms with P. putida, whereas wild-type cells persisted and overran the competitor. Our data show that T6SSs within a single organism can have distinct functions in eukaryotic versus bacterial cell interactions. These systems are likely to be a decisive factor in the survival of bacterial cells of one species in intimate association with those of another, such as in polymicrobial communities present both in the environment and in many infections.

### SUMMARY

Many bacteria encounter both eukaryotic cells and other bacterial species as a part of their lifestyles. In order to compete and survive, these bacteria evolved specialized pathways that target these distinct cell types. Type VI secretion systems (T6SSs) are bacterial protein export machines postulated to puncture targeted cells using an apparatus that shares structural similarity to bacteriophage. We investigated the role of the five T6SSs of Burkholderia thailandensis in the defense of the organism against other bacteria and higher organisms. B. thailandensis is a relatively avirulent soil saprophyte that is closely related to the human pathogen, B. pseudomallei. Our work uncovered roles for two B. thailandensis T6SSs with specialized functions in the survival of the organism in a murine host, or against another bacterial cell. We also found that B. thailandensis lacking the bacterial-targeting T6SS could not persist in a mixed biofilm with a competing bacterium. Based on the evolutionary relationship of T6SSs, and our findings that B. thailandensis engages other bacterial species in a T6S-dependent manner, we speculate that this pathway is of general significance to interbacterial interactions in polymicrobial human diseases and the environment.

### INTRODUCTION

Bacteria have evolved many mechanisms of defense against competitors and predators in their environment. Some of these, such as type III secretion systems (T3SSs) and bacteriocins, provide specialized protection against eukaryotic or bacterial cells, respectively [1,2]. Gene clusters encoding apparent type VI secretion systems (T6SSs) are widely dispersed in the proteobacteria; however, the general role of these systems in eukaryotic versus bacterial cell interactions is not known [3,4].

To date, most studies of T6S have focused on its role in pathogenesis and host interactions [5,6,7]. In certain instances, compelling evidence for the specialization of T6S in guiding eukaryotic cell interactions has been generated. Most notably, the systems of Vibrio cholerae and Aeromonas hydrophile were shown to translocate proteins with host effector domains into eukaryotic cells [8,9]. Evidence is also emerging that T6SSs could contribute to interactions between bacteria. The Pseudomonas aeruginosa HSI-I-encoded T6SS(H1-T6SS) was shown to target a toxin to other P. aeruginosa cells, but not to eukaryotic cells [10]. Unfortunately, analyses of the ecological niche occupied by bacteria that possess T6S have not been widely informative for classifying their function [3,4]. These efforts are complicated by the fact that pathogenic proteobacteria have environmental reservoirs, where they undoubtedly encounter other bacteria. The observation that many bacteria possess multiple evolutionarily distinct T6S gene clusters—up to seven in one organism—raises the intriguing possibility that each system may function in an organismal or context-specific manner [3].

The T6SS is encoded by approximately 15 core genes and a variable number of non-conserved accessory elements [4]. Data from functional assays and protein localization studies suggest that these proteins assemble into a multi-component secretory apparatus [11,12,13]. The AAA+ family ATPase, ClpV, is one of only a few core proteins of the T6S apparatus that have been characterized. Its ATPase activity is essential for T6S function [14], and it associates with several other conserved T6S proteins [15,16]. ClpV-interacting proteins A and B (VipA and VipB) form tubules that are remodeled by the ATPase, which could indicate a role for the protein in secretion system biogenesis. Two proteins exported by the T6SS are haemolysin co-regulated protein (Hcp) and valine-glycine repeat protein G (VgrG). Secretion of these proteins is co-dependent, and they may be extracellular components of the apparatus [10,13,17,18,19,20].

Burkholderia pseudomallei is an environmental saprophrophyte and the causative agent of melioidosis [21]. Infection with B. pseudomallei typically occurs percutaneously via direct contact with contaminated water or soil, however it can also occur through inhalation. The ecological niche and geographical distribution of B. pseudomallei overlap with a relatively non-pathogenic, but closely related species, Burkholderia thailandensis (B. thai) [22]. The genomes of these bacteria are highly similar in both overall sequence and gene synteny [23,24]. One study estimates that the two microorganisms separated from a common ancestor approximately 47 million years ago [24]. It is postulated that the B. pseudomallei branch then diverged from Burkholderia mallei, which underwent rapid gene loss and decay during its evolution into an obligate zoonotic pathogen [25]. As closely related organisms that represent three extremes of bacterial adaptation, the Burkholderia offer unique insight into the outcomes of different selective pressures on the expression and maintenance of certain traits.

B. pseudomallei possesses a large and complex repertoire of specialized protein secretion systems, including three type III secretion systems (T3SSs) and six evolutionarily distinct T6SSs [3,26,27]. The genomes of B. thailandensis and B. mallei contain unique sets of five of the six B. pseudomallei T6S gene clusters; thus, of the six evolutionarily distinct “Burkholderia T6SSs,” four are conserved among the three species. Remarkably, T6SSs account for over 2% of the coding capacity of the large genomes of these organisms. For the current study, we have adopted the Burkholderia T6SS nomenclature proposed by Thomas and colleagues [28].

To date, only Burkholderia T6SS-5, one of the four conserved systems, has been investigated experimentally. The system was investigated in B. mallei based on its co-regulation with virulence determinants such as actin-based motility and capsule [27]. B. mallei strains lacking a functional T6SS-5 are strongly attenuated in a hamster model of glanders. Preliminary studies suggest that T6SS-5 is also required for B. pseudomallei pathogenesis [28,29]. In one study, a strain bearing a transposon insertion within T6SS-5 was identified in a screen for B. pseudomallei mutants with impaired intercellular spreading in cultured epithelial cells [29]. The authors also showed that this insertion caused significant attenuation in a murine infection model.

Herein, we set out to systematically define the function of the Burkholderia T6SSs. Our study began with the observation that well characterized examples of eukaryotic and bacterial cell-targeting T6SSs segregate into distant subtrees of the T6S phylogeny. We found that Burkholderia T6SS-5 clustered closely with eukaryotic cell-targeting systems, and was the only system in B. thai that was required for virulence in a murine model of pneumonic melioidosis. The remaining systems clustered proximal to a bacterial cell-targeting T6SS in the phylogeny. One of these, T6SS-1, displayed a profound effect on the fitness of B. thai in competition with several bacterial species. The function of T6SS-1 required cell contact and its absence caused sensitivity of the strain to stasis induced by competing bacteria. In flow cell biofilm assays initiated with 1:1 mixtures of B. thai and Pseudomonas putida, wild-type B. thai predominated, whereas the ΔT6SS-1 strain was rapidly displaced by P. putida. Our findings point toward an important role for T6S in interspecies bacterial interactions.

### Results

### Phylogenetic Analysis of T6SSs

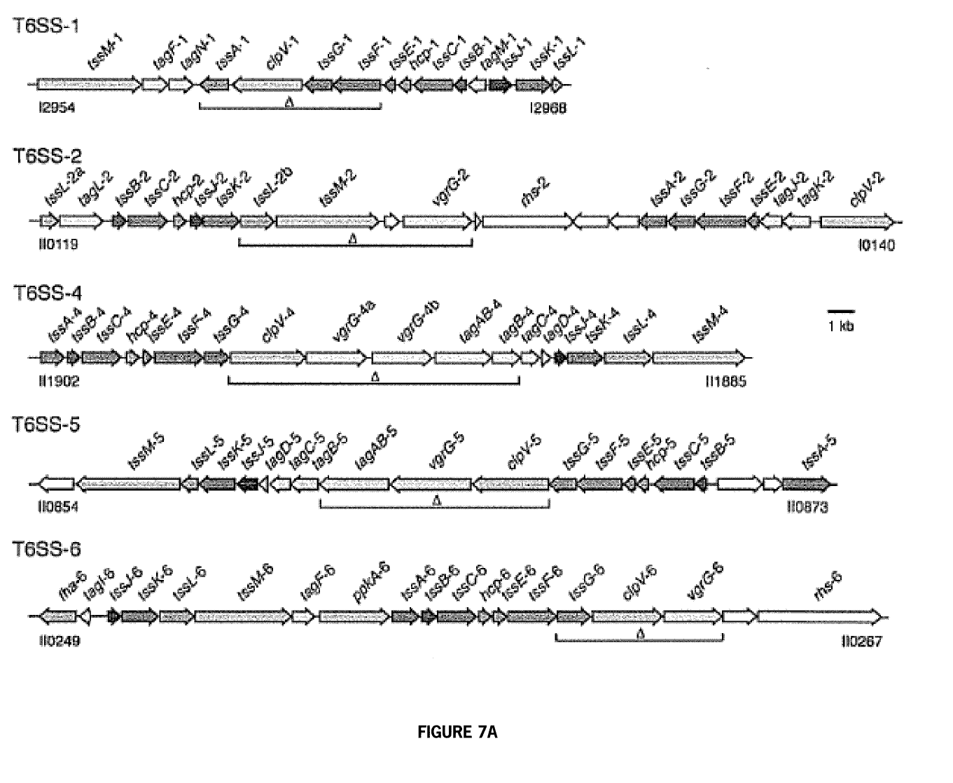

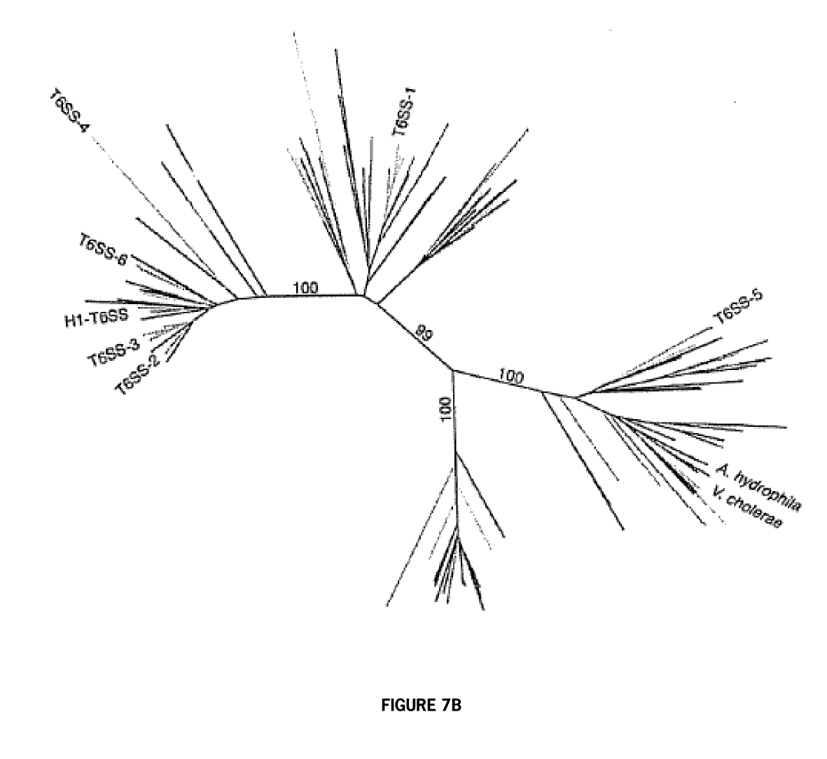

We conducted phylogenetic analyses of all available T6SSs to examine the evolutionary relationship between eukaryotic and bacterial cell-targeting systems. The phylogenetic tree we constructed was based on VipA, as this protein is a highly conserved element of T6SSs that has been demonstrated to physically interact with two other core T6S proteins, including the ClpV ATPase [15]. In the resulting phylogeny, the systems of V. cholerae and A. hydrophila, two well-characterized eukaryotic cell-targeting systems, clustered closely within one of the subtrees, whereas the bacteria-specific P. aeruginosa H1-T6SS was a member of a distant subtree (FIG. 7) [8,9,10]. In an independent analysis, Bingle and colleagues observed a similar T6S phylogeny, and termed these subtrees “D” and “A,” respectively {Bingle, 2008 #190}.

Next we examined the locations of the six Burkholderia T6SSs. Interestingly, T6SS-5, the only Burkholderia system previously implicated in virulence, clustered within the substree containing the V. cholerae and A. hydrophila systems (FIG. 7). Four of the remaining Burkholderia systems clustered within the subtree that included the H1-T6SS, and the final system was found in a neighboring subtree. These data led us to hypothesize that T6SSs of differing organismal specificities are evolutionarily distinct. Apparent contradictions between organismal specificity based on our phylogenetic distribution and studies demonstrating T6S-dependent phenotypes were identified, however these instances are difficult to interpret because specificity was not measured and cannot be ascertained from available data.

### T6SS-5 is Required for Virulence; Systems 1,2,4 and 6 are Dispensible

We chose B. thai as a tractable model organism in which to experimentally investigate the role of the Burkholderia T6SSs. Due to our limited knowledge regarding the function and essentiality of each gene within a given T6SS cluster, we reasoned it prudent to inactivate multiple conserved genes for initial phenotypic studies. Strains lacking the function of each of the five B. thai T6SSs (Burkholderia T6SS-3 is absent in B. thai) were prepared by removing three to five genes, including at least two that are highly conserved (FIG. 7A). When possible, polar effects were minimized by deleting from a central location in each cluster.

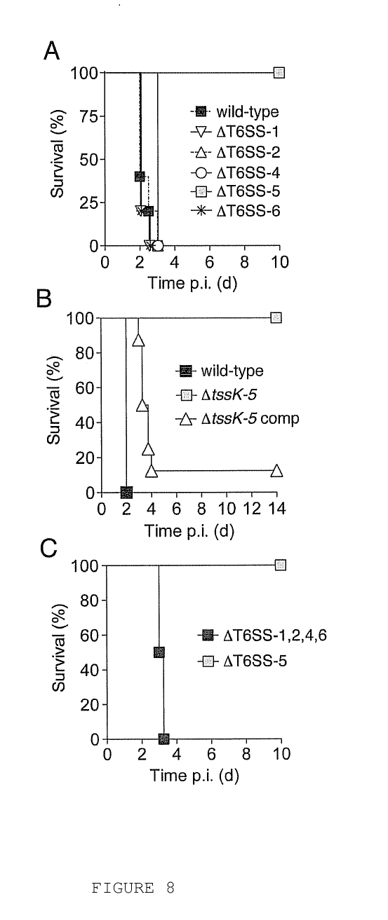

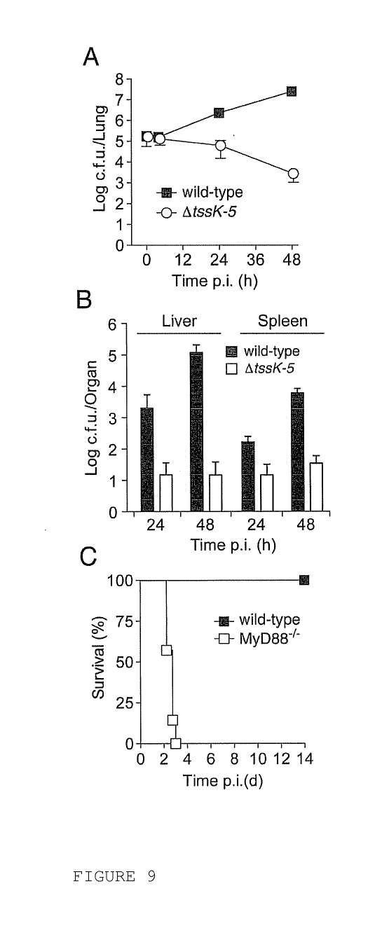

To probe the role of the Burkholderia T6SSs in virulence, we utilized a recently developed acute pneumonia model of melioidosis [30]. The survival of mice infected with approximately 10⁵ aerosolized wild-type or mutant bacteria was monitored over the course of ten days. Consistent with previous studies implicating T6SS-5 in B. mallei and B. pseudomallei pathogenesis, mice infected with ΔT6SS-5 survived the course and displayed no outward symptoms of the infection (FIG. 8A) {Pilatz, 2006 #124; Schell, 2007 #113}. On the other hand, those infected with the wild-type strain or strains bearing deletions in the other T6SSs succumbed by three days post infection (p.i.).

The B. thai T6SS-5 locus is adjacent to bsa genes, which encode an animal pathogen-like T3SS. Inactivation of the bsa T3SS secretion system also leads to dramatic attenuation of B. thai in the model we utilized [26]. The regulation of these secretion systems appears to be intertwined; a recent study in B. pseudomallei showed that a protein encoded within the bsa cluster strongly activates T6SS-5 of that organism [31]. To rule out the possibility that attenuation of ΔT6SS-5 was attributable to polar effects or changes in regulation of the bsa T3SS, we generated a strain bearing an in-frame deletion of a single gene in the cluster, tssK-5 (FIG. 7A). A tssK-5 ortholog is readily identified in nearly all T6S gene clusters and it shares no homology with known regulators. Like the T6SS-5 deletion, ΔtssK-5 completely attenuated the organism (FIG. 8B). Genetic complementation of this phenotype further confirmed that T6SS-5 is an essential virulence factor of the organism.

To investigate whether the retention of virulence in the ΔT6SS-1,2,4 and 6 strains could be attributed to either compensatory activity or redundancy, we next constructed a strain bearing inactivating mutations in all four clusters and measured its virulence in mice. Mice infected with this strain succumbed to the infection with similar kinetics to those infected with the wild-type, indicating that T6SS-5 is the only system of B. thai that is required for virulence in this model (FIG. 8C). In summary, these data indicate that T6SS-5 is a major virulence factor for B. thai in a murine acute melioidosis model, whereas the remaining putative T6SSs of the organism are dispensible for virulence.

### Burkholderia T6SS-5 Plays a Specific Role in Host Interactions

To more closely examine the requirement for T6SS-5 during infection, we monitored B. thai wild-type and ΔtssK-5 c.f.u. in the lung, liver, and spleen at 4, 24, and 48 hrs following inoculation with approximately 10⁵ bacteria by aerosol. At 4 hrs p.i., no differences were observed in c.f.u. recovered from the lung (FIG. 9A). After this initial phase, lung c.f.u. of ΔtssK-5 gradually declined, whereas wild-type populations expanded approximately 100-fold. Both organisms spread systemically, however significantly fewer ΔtssK-5 cells were recovered from the liver and spleen at 24 and 48 hrs p.i. (FIG. 9B).

Thus far, our findings did not distinguish between a specific role for T6SS-5 in host interactions, such as escaping or manipulating the innate immune system, versus the alternative explanation that T6SS-5 is generally required for growth in host tissue. To discriminate between these possibilities, we compared the virulence of ΔtssK-5 in wild-type mice to a strain with compromised innate immunity, MyD88^{-/-} [32,33]. Mice lacking MyD88 were unable to control the ΔtssK-5 infection and succumbed within 3 days (FIG. 9C). The differences in virulence of the Δtssk-5 strain in wild-type and MyD88−/− infections suggest that T6SS-5 is required for effective defense of the bacterium against one or more innate responses of the host. Altogether, these data strongly support the conclusion that T6SS-5 has evolved to play a specific role in the fitness of B. thai in a eukaryotic host environment.

T6S Impacts the Fitness of B. Thai in Co-Culture with Diverse Bacterial Species

Earlier work by our laboratory has shown that T6S can influence intraspecies bacterial interactions. We showed that the H1-T6SS of P. aeruginosa targets a toxin to other P. aeruginosa cells [10], and that in growth competition assays, toxin-secreting strains are provided fitness advantage relative to strains lacking a specific toxin immunity protein. Based on this information and the locations of the B. thai T6SSs within our phylogeny, we postulated that one or more of these systems could also play a role in interbacterial interactions. Preliminary studies indicated that T6S did not influence interactions between B. thai strains, thus we decided to test the hypothesis that the B. thai T6SSs play a role in interspecies bacterial interactions.

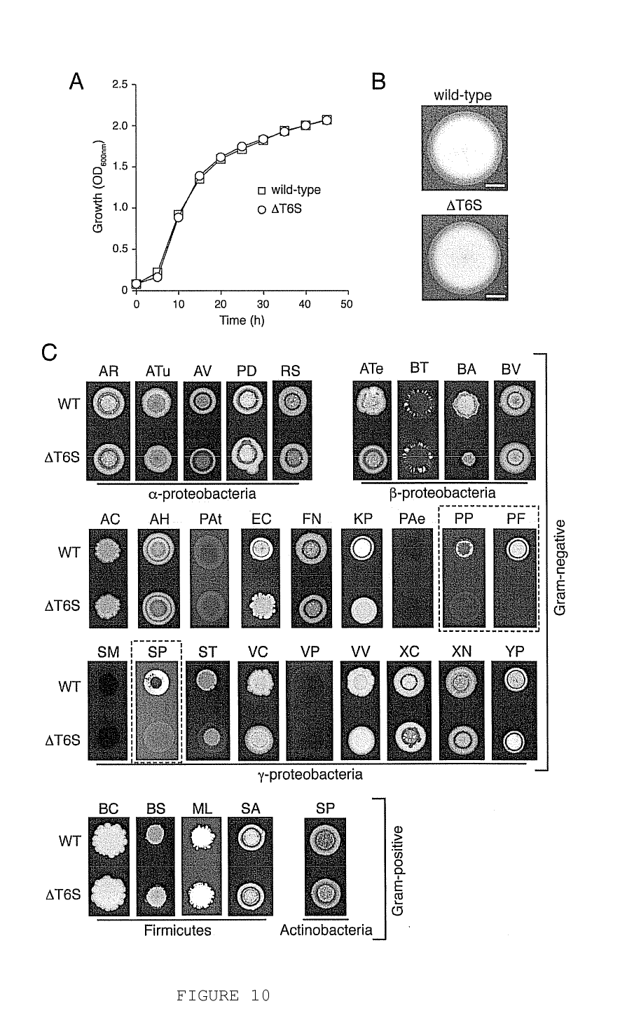

Without information to guide predictions of specificity, we developed a simple and relatively high-throughput semi-quantitative assay to allow screening of a wide range of organisms for sensitivity to the B. thai T6SSs. The design of the assay was based on two key assumptions for T6S-dependent effects—that they are cell contact-dependent and that they impact fitness (as measured by proliferation). To facilitate measurement of T6S-dependent changes in B. thai proliferation in the presence of competing organisms, we engineered constitutive green fluorescent protein expression cassettes into wild-type B. that and a strain bearing mutations in all five T6SSs (ΔT6S) [34]. Control experiments showed that the lack of T6S function did not impact growth or swimming motility (FIGS. 10A and 10B). To test the assay, we conducted competition experiments between the GFP-labeled wild-type and ΔT6S strains against the unlabeled wild-type strain. The GFP-expressing cells were clearly visualized in the mixtures, and, importantly, wild-type and ΔT6S competed equally with the parental strain (FIG. 10C; BT).

We next screened the B. thai strains against 31 species of bacteria. Most of these were Gram-negative proteobacteria (5α; 3β; 18γ), however two Gram-positive phyla were also represented (4 Firmicutes; 1 Actinobacteria). Although we endeavored to screen a large diversity of bacteria, many taxa could not be included due to specific nutrient requirements or an unacceptably slow growth rate under the conditions of the assay (30° C., Luria-Bertani (LB) medium). The outcomes of most competition experiments were independent of the T6SSs of B. thai. T6S-independent outcomes varied; in most instances, B. thai flourished in the presence of the competing organism (FIG. 10C). However, a small subset of species markedly inhibited B. thai growth (FIG. 10C; ECa, PA, SM, VP). Interestingly, B. thai proliferation was reproducibly affected in a T6S-dependent manner in competition experiments against 7 of the 31 species tested. All of these were Gram-negative organisms, and in each case, B. thai ΔT6S was less fit than the wild-type. T6S-dependent competition outcomes fell into two readily discernable groups; the first included three γ- and one β-proteobacteria (FIG. 10C; BA, ECo, KP, ST). In competition with these organisms, B. thai ΔT6S displayed only a modest decrease in proliferation relative to the wild-type. Differences in the size and morphology of assay “spots” containing wild-type or ΔT6S were noted in several instances for this group of organisms. Quantification of c.f.u. verified that these differences were reflective of a minor, but highly reproducible fitness defect of AT6S (data not shown).

The second group consisted of three γ-proteobacteria, P. putida, P. fluorescens, and S. proteamaculans. The proliferation of B. thai grown in competition with these organisms appeared to be highly dependent on T6S (FIG. 10F; PP, PF, SP). For further analyses, we focused on this latter group; henceforth refer to as the “T6S-dependent competitors” (TDCs).

### T6SS-1 is Involved in Cell Contact-Dependent Interbacterial Interactions

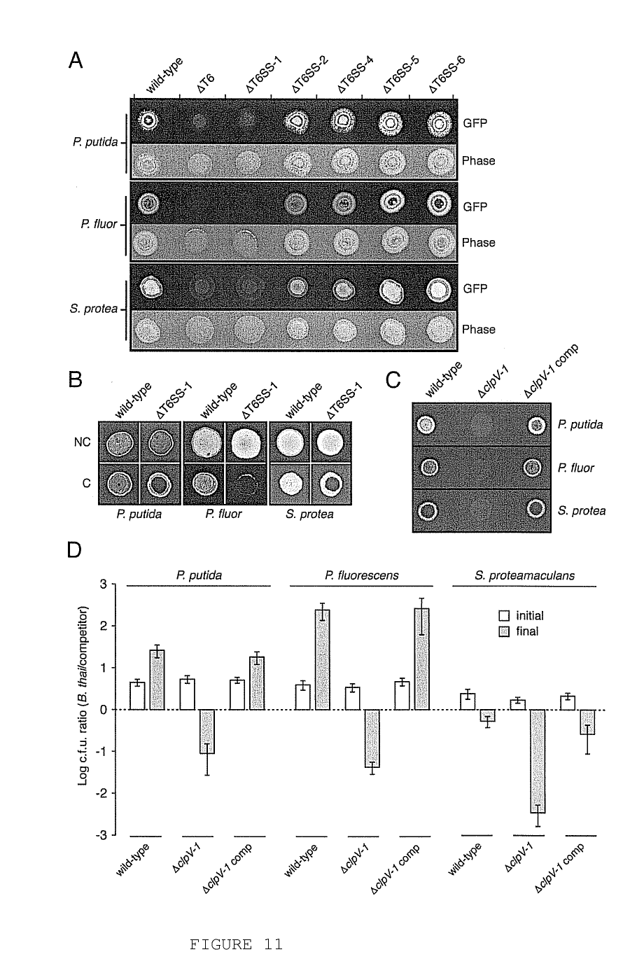

The next question we addressed was whether one or more of the individual T6SSs were responsible for the TDC-specific proliferation phenotype of B. thai ΔT6S. To determine this, we inserted a GFP over-expression cassette into our panel of individual B. thai T6SS deletion strains, and performed plate competition assays against the TDCs. In competition with each TDC, ΔT6SS-1 appeared as deficient in proliferation as ΔT6S, whereas the other strains grew similarly to the wild-type (FIG. 11A). The dramatic differences in the competitions outcomes between the strains were also discernable by the naked eye. Competition experiments that included B. thai lacking T6SS-1 had a morphology similar to a mono-culture of the TDC, whereas co-cultures possessing an intact T6SS-1 were more similar in appearance to B. thai mono-culture.

It remained possible that the effects of T6SS-1 on the fitness of B. thai in competition with other bacteria were either non-specific or unrelated to its putative role as a T6SS. As mentioned earlier, one common observation from detailed studies of T6SSs conducted to date is that its effects require cell contact [8,9,10]. This has been postulated to reflect a conserved mechanism of the apparatus akin to bacteriophage cell puncturing [18]. To address whether the apparent fitness defect of ΔT6SS-1 involves a mechanism consistent with T6S, we probed whether its effects were dependent upon cell contact. A filter (0.2 nm) placed between B. thai and TDC cells abrogated the T6SS-1-dependent growth defect (FIG. 11B). In control experiments, the three TDCs were directly applied to an underlying layer of the B. thai strains. In each case, a zone of clearing was observed in the ΔT6SS-1 layer, while no effect on wild-type proliferation was noted. From these data we conclude that cell contact is essential for the activity of T6SS-1.

We next sought to quantify the magnitude of T6SS-1 effects on B. thai fitness in competition with TDCs. To ensure the specificity of T6SS-1 inactivation in the strains used in these assays, we generated a B. thai strain bearing an in-frame clpV-1 deletion, and a strain in which this deletion was complemented by clpV-1 expression from a neutral site on the chromosome. In plate competition assays, the ΔclpV-1 strain displayed a fitness defect similar to ΔT6SS-1, and clpV-1 expression complemented the phenotype (FIG. 11C). Measurements comparing B. thai and TDC c.f.u. in the competition assay inoculum to material recovered from the assays following several days of incubation confirmed that inactivation of T6SS-1 leads to a dramatic fitness defect of B. thai (FIG. 11D). Depending on the TDC, the competitive index (c.i.; final c.f.u. ratio/initial c.f.u ratio) of wild-type B. thai was approximately 120-5.000-fold greater than that of the ΔclpV-1 strain. All TDCs out-competed ΔclpV-1 (0.0021<c.i.<0.015); on the contrary, wild-type B. thai was highly competitive against P. putida (c.i.: 5.8) and P. fluorescens (c.i.: 61), and its relative numbers decreased only modestly in assays with S. proteamaculans (c.i.: 0.24). In summary, our findings indicate that T6SS-1 plays an important role in the interactions of B. thai cells in direct contact with other bacteria. T6SS-1-dependent effects are species-specific, and in some cases, can be a major determinant of B. thai proliferation.

### T6SS-1 Provides Resistance to P. Putida Induced Stasis of B. Thai

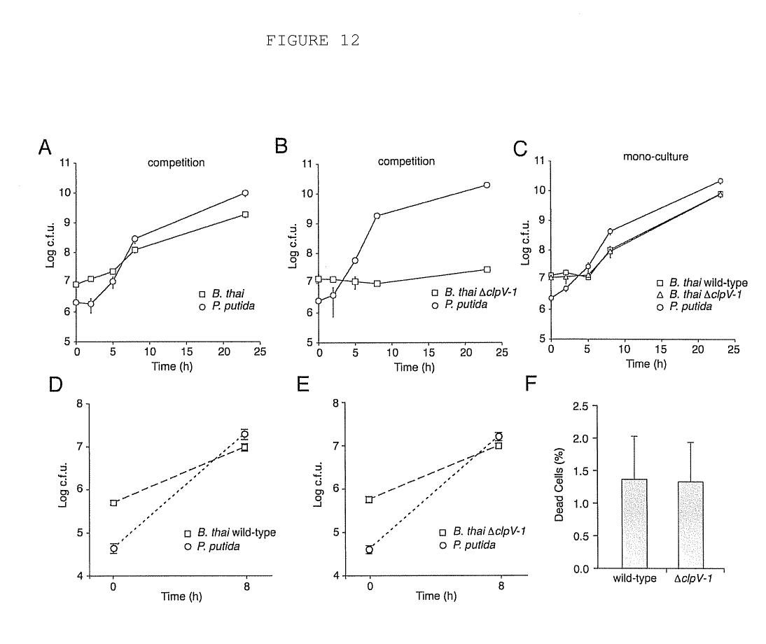

Three models could explain the T6SS-1-dependent effects we observed on B. thai fitness in competition with the TDCs: (i) T6SS-1 inhibits TDC proliferation, thereby freeing nutrients for B. thai (ii) T6SS-1 prevents TDC inhibition of B. thai growth, or (iii) T6SS-1 performs both of these functions. To distinguish between these possibilities, we compared B. thai and TDC growth rates following inoculation into either mono-culture or competitive cultures on 3% agar plates. Our prior experiments indicated that T6SS-1-dependent effects on B. thai were similar in competition assays with each TDC (FIG. 10F and FIG. 11), therefore we utilized P. putida to represent the TDCs in this and subsequent experiments. Surprisingly, we found that the proliferation of P. putida and wild-type B. thai was largely unaffected in competition assays (FIG. 12A-C). However, ΔclpV-1 proliferation was severely hampered in the presence of P. putida. Indeed, B. thai ΔclpV-1c.f.u. expanded by only 2.1-fold during the first 23 hours of the experiment, whereas wild-type c.f.u. increased 220-fold. Consistent with earlier results in P. aeruginosa {Hood, 2010 #333}, the effects of T6SS-1 on the fitness of B. thai in co-culture with P. putida were not observed in liquid medium (FIGS. 12D and 12E).

The proliferation defect of B. thai ΔclpV-1 could be attributable to P. putida-induced growth inhibition, cell killing, or a combination of these factors. We reasoned that if killing was involved in the ΔclpV-1 phenotype, the difference in cell death between wild-type and ΔclpV-1 would be most pronounced at approximately 7.5 hrs following inoculation of the competition assays, when wild-type B. thai are rapidly proliferating and ΔclpV-1 cell numbers are not expanding. At this time point, we identified similar numbers of dead cells in wild-type and ΔclpV-1 competitions, suggesting that T6SS-1 inhibits stasis of B. thai induced by P. putida (FIG. 12F).

T6SS-1 is Required for the Persistence of B. Thai in Mixed Biofilms with P. Putida

In our plate competition assays, low moisture availability impairs bacterial motility, and artificially enforces close association of B. thai with the TDCs. To determine whether T6SS-1 could provide a fitness advantage for B. thai under conditions more relevant to its natural habitat, i.e., where nutrients are exchanged and dehydration does not drive interbacterial adhesion, we conducted mixed species flow chamber biofilm assays.

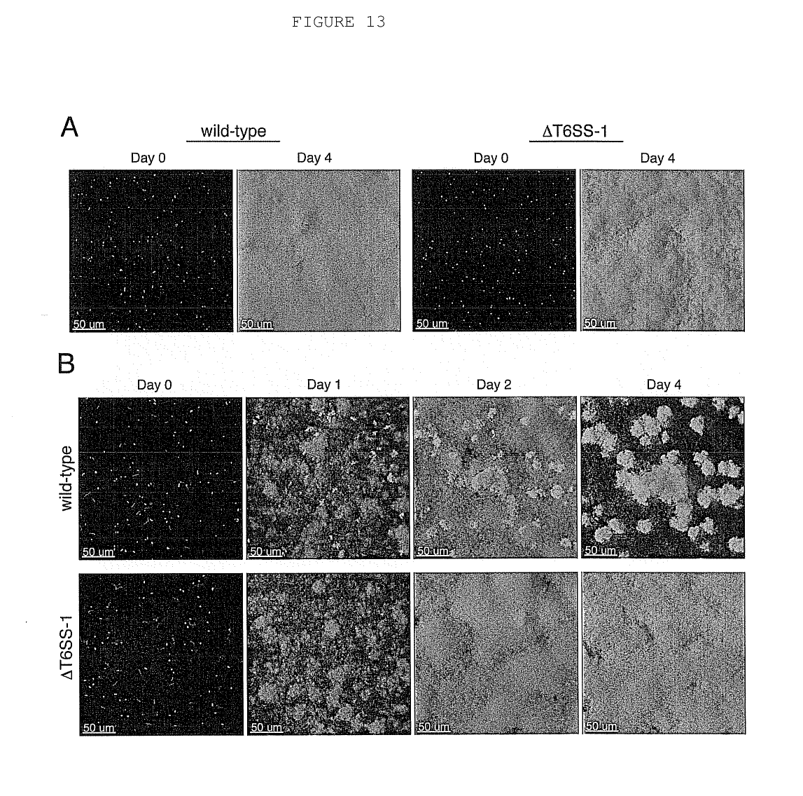

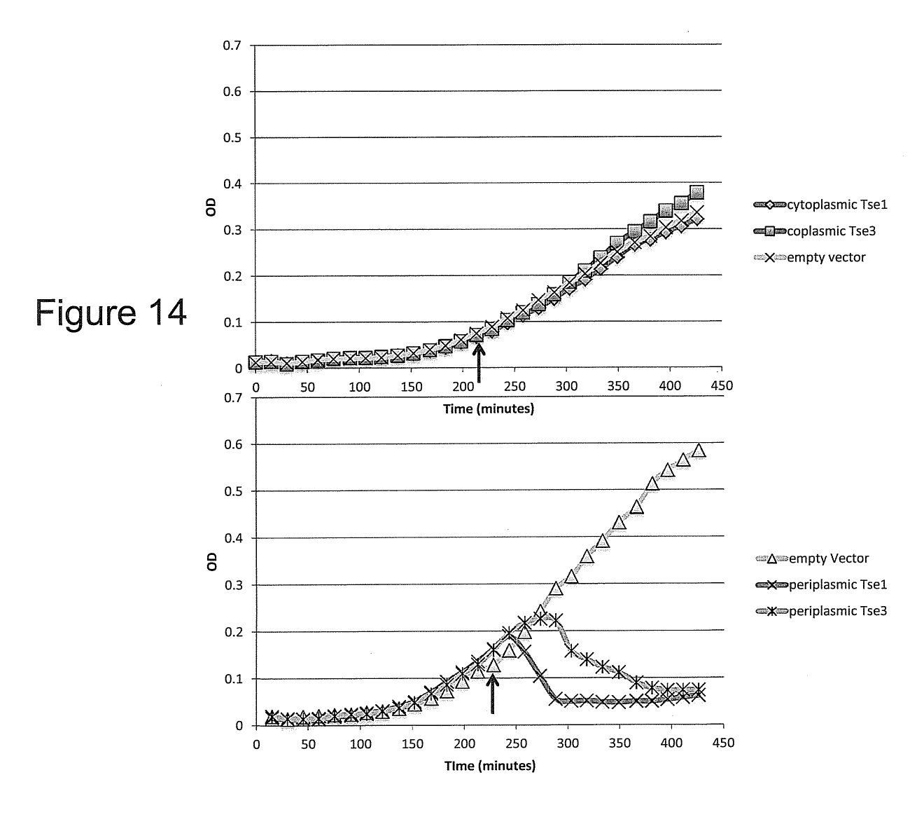

Previous studies in E. coli and V. parahaemolyticus have implicated T6S in the inherent capacity of these organisms to form biofilms {Aschtgen, 2008 #224; Enos-Berlage, 2005 #58}. Furthermore, additional T6SSs are activated during biofilm growth or co-regulated with characterized biofilm factors such as exopolysaccharides {Aubert, 2008 #191; Deretic, 1995 #288; Mougous, 2006 #87; Sauer, 2002 #395; Southey-Pillig, 2005 #396}. Thus, prior to performing mixed species assays, we first tested whether inactivation of T6SS-1 influenced the formation of monotypic B. thai biofilms. Wild-type and ΔT6SS-1 strains adhered equally to the substratum and formed indistinguishable monotypic biofilms that reached confluency after four days (FIG. 13A), indicating T6SS-1 does not play a role in the inherent ability of B. thai to form biofilms.

Next we seeded biofilm chambers with 1:1 mixtures of B. thai and P. putida. In mixed biofilms, the B. thai strains again adhered with similar efficiency, however a dramatic difference between the capacity of the strains to persist and proliferate in the presence of P. putida became apparent within 24 hrs (FIG. 13B). At this time point, wild-type B. thai microcolonies had expanded and its cells were dispersed throughout the P. putida-dominated biofilm, whereas B. thai ΔclpV-1 microcolonies had diminished in number. Consistent with the results of our plate assays, P. putida growth was not noticeably impacted by the activity of T6SS-1 at early time points in the experiment. As the biofilm matured, wild-type B. thai gradually displaced P. putida, and by four days after seeding, B. thai microcolonies accounted for most of the biofilm volume. These data suggest that T6SS-1 can provide a major fitness advantage for B. thai in interspecies biofilms.

### Discussion

Our findings suggest that the highly conserved T6S architecture can serve diverse functions. We found T6SSs within B. thai critically involved in two very distinct processes—virulence in a murine infection model and growth in the presence of specific bacteria. The systems involved in these diverse phenotypes, T6SS-5 and T6SS-1, respectively, are distantly related, and cluster phylogenetically with other T6SSs of matching cellular specificity. We were unable to define the function for three of the B. thai T6SSs, however their clustering in the H1-T6SS subtree suggests that they could have a role in interbacterial interactions. These systems may not have been active under the assay conditions we utilized, they might be specific for organisms we did not include in our screen, or their activity may not affect proliferation. Phylogenies have proven to be powerful tools for guiding researchers studying complex protein secretion systems [35,36]. However, determining whether T6S phylogeny holds promise as a general predictor of organismal specificity will require more studies that evaluate the significance of individual systems in both eukaryotic and bacterial cell interactions.

Although B. thai is not generally regarded as a pathogen, our data suggest that Burkholderia T6SS-5 plays a role in host interactions that is conserved between this species and its pathogenic relatives, B. pseudomallei and B. mallei [27,28,29,37]. We postulate that T6SS-5, like many other virulence factors, evolved to target simple eukaryotes in the environment. The benefit T6SS-5 provides the Burkholderia in a mammalian host could have been one factor that allowed B. mallei to transition into an obligate pathogen. Based on our results implicating T6SS-1 exclusively in interbacterial interactions, the role of this system in the lifestyle of B. mallei is more difficult to envisage. Indeed, the cluster encoding T6SS-1 is the most deteriorated of the T6S clusters of B. mallei and is unlikely to function [27]. Of the 13 conserved T6S-associated orthologous genes, 8 of these appear to be deleted in B. mallei T6SS-1, however the remaining T6S clusters of the organism are largely intact (0-3 pseudogenes or absent genes).

Of the 33 organisms screened, the effects of B. thai T6SS-1 were most pronounced in competitions with P. putida, P. fluorescens, and S. proteamaculans. Whether these organisms are physiologically relevant B. thai T6SS-1 targets is not known, however P. putida and P. fluorescens have been isolated from soil in Thailand [38,39], and the capacity of these organisms to form biofilms is well documented [40,41,42]. P. putida and P. fluorescens are recognized biological control agents, suggesting that the rhizosphere could be one habitat where antagonism with B. thai might occur [43]. Notably, we did not observe T6SS-dependent effects on B. thai proliferation in the presence of the five Gram-positive organisms included in our screen. The number and diversity of organisms we tested were too low to ascribe statistical significance to this observation, however it is tempting to speculate that the effects of T6S might be limited to Gram-negative cells. This would not be unexpected given the structural relatedness of T6S apparatus components to the puncturing device of T4 bacteriophage [18,19,20].

We found that T6SS-1 allows B. thai to proliferate in the presence of the TDCs. This surprising and counterintuitive finding raises the question of what inhibits B. thai ΔclpV-1 growth, and is it an intrinsic (derived from B. thai) or extrinsic (derived from the TDC) factor? Our data indicate that the activity or production of this factor manifests in the absence of T6SS-1 function only when a TDC is present and intimate cell contact occurs. If the factor is intrinsic, we postulate that its activity is inappropriately triggered by ΔT6SS-1 in the presence of the TDCs, but that its function serves an adaptive role for wild-type B. thai. For example, under circumstances where it is not advantageous for B. thai to proliferate, such as when it is exposed to particular organisms, antibiotics, or stresses, this factor could initiate dormancy. There is evidence that T6S components can participate in cell-cell recognition in bacteria. Gibbs et al. recently reported the discovery of an “identification of self” (ids) gene cluster within Proteus mirabilis that contains genes homologous to hcp (idsA) and vgrG (idsB) {Gibbs, 2008 #327}. Inactivation of idsB caused a defect in recognition of its parent, resulting in boundary formation between the strains.

If the factor is extrinsic, T6SS-1 might be more appropriately defined as a defensive, rather than an offensive pathway. T6SS-1 could provide defense by either influencing the production of the extrinsic factor within the TDC, such as by repressing expression, or it could provide physical protection against the factor by obstructing or masking its target. If the fitness effect that T6SS-1 provides B. thai depends on a specific offensive pathway present in competing organisms, the presence of this pathway in an organism could be the basis for the apparent specificity we observed in our screen. Future studies must address whether the determinants of T6SS-1 effects are intrinsic, extrinsic, or a combination of the two. The design of our competition screen was limited in this regard; we measured T6SS-1 activity indirectly, and we were able to test only a modest number of species. Understanding the mechanism of action of T6SS-1, for example by identifying its substrates, will provide insight into the specificity of the secretion apparatus.

While it is widely accepted that diffusible factors such as antibiotics, bacteriocins, and quorum sensing molecules are common mediators of dynamics between species of bacteria, an analogous cell contact-dependent pathway has yet to be defined [44]. We found that T6S can provide protection for a bacterium against cell contact-induced growth inhibition caused by other species of bacteria. Given that most organisms that possess T6S gene clusters are either opportunistic pathogens with large environmental reservoirs or strictly environmental organisms, we hypothesize that T6SSs are, in fact, widely utilized in interbacterial interactions. Bacteria-targeting T6SSs may be of great general significance to understanding interactions and competition within bacterial communities in the environment and in polymicrobial infections.

### Materials and Methods

### Ethics Statement

All research involving live animals was conducted in compliance with the Animal Welfare Act and other federal statutes and regulations relating to animals and experiments involving animals, and adhered to the principles stated in the Guide for the Care and Use of Laboratory Animals, National Research Council, 1996. All work involving animals was approved by the Institutional Animal Care and Use Committee at the University of Washington.

### Strains and Growth Conditions

B. thai E264 and E. coli cloning strains were routinely cultured in Luria-Bertani (LB) broth or on LB agar at 37° C. All bacterial species used in this study are listed in the legend of FIG. 10. The medium was supplemented with trimethoprim (200 μg/ml), ampicillin (100 μg/ml), zeocin (2000 μg/ml), irgasan (25 μg/ml) or gentamicin (15 μg/ml) where necessary. For introducing in-frame deletions, B. thai was grown on M9 minimal medium agar plates with 0.4% glucose as a carbon source and 0.1% (w/v) p-chlorophenylalanine for counter selection [45].

Construction of Markerless in-Frame Deletions of T6SS Genes

B. thai T6SSs were inactivated utilizing a previously described mutagenesis technique based on the suicide plasmid pJRC 115 containing a mutated phenylalanine synthetase (pheS) gene for counterselection [45]. Unmarked in-frame deletions of three to five T6SS genes per T6SS gene cluster (at least two of which are core T6SS genes; see FIG. 7) were constructed by splicing by overlap PCR of flanking DNA [46]. The open reading frames were deleted except for 4-8 codons at the 5′ end of the upstream gene and 3′ end of downstream gene, and the insertional sequence TTCAGCATGCTTGCGGCTCGAGTT (SEQ ID NO:53) was added as previously described [14]. E. coli SM10 Xpir was used to deliver the deletion constructs into B. thai by conjugational mating and transconjugants were selected on LB agar plates supplemented with trimethoprim and irgasan.

Genetic Complementation of ΔtssK-5 and ΔclpV-1

The conserved T6SS genes tssK-5 (BTH_II0857) and clpV-1 (BTH_I2958) were deleted using the in-frame deletion mutagenesis technique described above. For single copy complementation, the mini-Tn7 system was utilized [34]. For this, the B. thai ribosomal promoter P_{S12} sequence was cloned into the suicide vector pUC18T-mini-Tn7T-Tp using complementary oligonucleotides to yield pUC18T-mini-Tn7T-Tp-P_{S12 [}47]. The tssK-5 and clpV-1 open reading frames along with 16-20 bp upstream were amplified and inserted into pUC18T-mini-Tn7T-Tp-P_{S12}. The resulting plasmids and the Tn7 helper plasmid, pTNS3, were introduced into appropriate deletion strains by electroporation using a previously described protocol [45,47]. Transposition of the Tn7-constructs into the chromosome of B. thai was determined by PCR as described previously[48].

### Construction of Fluorescently Labeled B. Thai and P. Putida

The mini-Tn7 system was utilized to integrate green fluorescent protein (GFP) and cyan fluorescent protein (CFP) expression cassettes into the chromosome of B. thai and P. putida, respectively [48,49]. To construct a mini-Tn7 derivative for constitutive expression of GFP, the GFP cassette was amplified from pQB1-T7-GFP (Quantum Biotechnologies) without the T7 promoter region as previously described and inserted into KpnI and Stul sites of pUC18T-mini-Tn7T-Tp-P_{S12 [}27]. This plasmid was then introduced into relevant B. thai strains and insertion of Tn7-GFP into the chromosome was verified as described above. To construct a GFP labeled ΔclpV-1 comp strain we made use of the fact that two Tn7 insertion sites (attTn7) are present in the genome of B. thai. The chromosomally integrated Tn7 Tp^{r} resistance cassette of ΔclpV-1 comp was excised using pFLPe2 which expresses a Flp recombinase (Choi, 2008) before introducing pUC18T-mini-Tn7T-Tp-P_{S12}-GFP. Insertion of Tn7-GFP into the other attTn7 site was confirmed by PCR as described previously [48,49]. To engineer CFP labeled P. putida, the mini-Tn7(Gm)-CFP plasmid and the helper plasmid pUX-BF13 were introduced into the strain by electroporation as previously described [49].

### In Vitro Growth Kinetics

Growth kinetics of B. thai strains were measured in LB broth using the automated BioScreen C Microbiology plate reader (Growth Curves) with agitation at 37° C. Three independent measurements were performed in triplicate for each strain.

### Swimming Motility Assays

Swimming motility of B. thai strains was analyzed in 0.25% LB agar. Swimming plates were stab-inoculated with overnight cultures and incubated at 37° C. for 48 h. Two independent experiments were performed.

Murine Infection Model.

Specific-pathogen-free C57BL/6 mice were obtained from Jackson Laboratories (Bar Harbor, Me.). MyD88^{-/-} mice were derived by Dr. Shizuo Akira (University of Osaka) and backcrossed for at least 8 generations to C57BL/6 [50]. Mice were housed in laminar flow cages with ad lib access to sterile food and water. The Institutional Animal Care and Use Committee of the University of Washington approved all experimental procedures. For aerosol infection of mice, bacteria were grown in LB broth at 37° C. for 18 hours, isolated by centrifugation, washed twice, and suspended in Dulbecco's PBS to the desired concentration. An optical density of 0.20 at 600 nm yielded approximately 1×10⁸ CFU/ml. Mice were exposed to aerosolized bacteria using a nose-only inhalation system (In-Tox Products, Moriarty, N. Mex.) (West, Trans R Soc Trop Med Hyg, 2008). Aerosols were generated from a MiniHEART hi-flo nebulizer (Westmed, Tucson, Ariz.) driven at 40 psi. Airflow through the system was maintained for 10 minutes at 24 l/min followed by five minutes purge with air. Immediately following aerosolization, the pulmonary bacterial deposition was determined by quantitative culture of left lung tissue from three to four sentinel mice. Following infection, animals were monitored one to three times daily for illness or death. Ill animals meeting defined clinical endpoints were euthanized. At specific time points after infection, mice were euthanized in order to quantify bacterial burdens and inflammatory responses. To determine bacterial loads, the left pulmonary hilum was tied off and the left lung, median hepatic lobe, and spleen each were removed and homogenized in 1 ml sterile Dulbecco's PBS. Serial dilutions were plated on LB agar and colonies were counted after 2-4 days of incubation at 37° C. in humid air under 5% CO₂.

### Interbacterial Growth Competition Assays

Overnight cultures of B. thai and competitor bacteria were adjusted to an OD₆₀₀ of 0.1 and mixed 5:1 (v/v). For competitions using fluorescent strains, 2.5 μl of the mixture was spotted on 3% w/v LB agar and fluorescence was measured after approximately one week following incubation at 30° C. For quantitative competitions using non-fluorescent strains, 10 μl of the mixture was spotted on a filter (0.22 μm; GE Water & Process Technologies) and cells were harvested and enumerated at the indicated time points. Colonies of the competing organisms were distinguished from B. thai strains using a combination of colony morphology, growth rate and inherent antibiotic susceptibility.

### Live/Dead Staining of Bacterial Cells

Growth competitions of B. thai against P. putida were performed on filters as described above. At 7.5 h after initiating the experiment, the filters were resuspended in 200 μl LB broth and cell viability was measured using the LIVE/DEAD BacLight Bacterial Viability Kit for microscopy according to the manufacturer's protocol (Invitrogen). The number of dead cells was determined for five random fields per competition using fluorescence microscopy. Two independent experiments were performed in duplicate.

### Flow-Chamber Biofilm Experiments

Biofilms were grown at 25° C. in three-channel flow-chambers (channel dimensions of 1×4×40 mm) irrigated with FAB medium supplemented with 0.3 mM glucose. Flow-chamber biofilm systems were assembled and prepared as previously described [51]. The substratum consisted of a 24×50 mm microscope glass cover slip. Overnight cultures of the relevant strains were diluted to a final OD₆₀₀ₙₘ of 0.01 in 0.9% NaCl, and 300 μl of the diluted bacterial cultures, or 1:1 mixtures, were inoculated by injection into the flow chambers. After inoculation, the flow chambers were allowed to stand inverted without flow for 1 h, after which medium flow was started with flow chambers standing upright. A peristaltic pump (Watson-Marlow 250S) was used to keep the medium flow at a constant velocity of 0.2 mm/s in the flow-chamber channels. Microscopic observation and image acquisition of the biofilms were performed with a Leica TCS-SP5 confocal laser scanning microscope (CLSM) (Leica Microsystems, Germany) equipped with lasers, detectors and filter sets for monitoring GFP and CFP fluorescence. Images were obtained using a 63×/1.4 objective. Image top-down views were generated using the IMARIS software package (Bitplane AG). The flow-chamber experiment reported here was repeated twice, and in each experiment each mono-strain or mixed-strain biofilm was grown in at least two channels, and at least 6 CLSM images were recorded per channel at random positions. Each individual image presented here is therefore representative of at least 24 images.

### T6S Phylogenetic Tree Construction

Annotated genomes were downloaded from the Genome Reviews ftp site (ftp://ftp.ebi.ac.uk/pub/databases/genome_reviews/, January 2010, 926 bacterial genomes (1814 chromosomes and plasmids) [52]. Protein sequences from all genomes were aligned with rpsblast [53] against the COG section of the CDD database (January 2010) [54]. Only proteins showing an alignment covering at least 30% of the COG PSSM with an E-value≦10⁻⁶ were retained. To avoid any errors in COG assignments, we discarded all hits that overlap with another hit with a better E-value on more than 50% of its length. We considered the following 13 COGs as ‘T6SS core components’: COG0542, COG3157, COG3455, COG3501, COG3515, COG3516, COG3517, COG3518, COG3519, COG3520, COG3521, COG3522, COG3523 [3,4]. Two genes were considered neighbours if they are separated by less than 5000 bp. Only clusters containing the VipA protein (COG3516) and genes encoding for at least five other T6SS core components were included in the analyses. The Edwardsiella tarda (EMBL access AY424360) system was added manually because the complete genome sequence and annotation of this organism was unavailable in Genome Reviews.

In three of the 334 T6SS clusters, two VipA coding genes were identified. Manual inspection of two of these clusters in Acinetobacter baumannii (ATCC 17978) and Vibrio cholerae (ATCC 39541) revealed that they resulted from apparent gene fissions; in both cases we kept the longest fragment corresponding to the C-terminal part of the full length protein. In the third case, Psychromonas ingrahamii (strain 37), the two VipA coding genes resulted from an apparent duplication event: one of the two copies showed a high mutation frequency and was discarded. In total, we included 334 VipA orthologs in T6SS clusters. The 334 VipA protein sequences were aligned using muscle [55]. Based on this alignment, a neighbour-joining tree with 100 bootstrap replicates was computed using BioNJ [56].

### REFERENCES FOR EXAMPLE 3

1. Riley M A, Wertz J E (2002) Bacteriocins: evolution, ecology, and application Annu Rev Microbiol 56: 117-137.
2. Cornelis G R (2006) The type III secretion injectisome. Nat Rev Microbiol 4: 811-825.
3. Bingle L E, Bailey C M, Pallen M J (2008) Type VI secretion: a beginner's guide. Curr Opin Microbiol 11:3-8.
4. Boyer F, Fichant G, Berthod J, Vandenbrouck Y, Attree I (2009) Dissecting the bacterial type VI secretion system by a genome wide in silico analysis: what can be learned from available microbial genomic resources? BMC Genomics 10: 104.
5. Cascales E (2008) The type VI secretion toolkit. EMBO Rep 9: 735-741.
6. Filloux A, Hachani A, Bleves S (2008) The bacterial type VI secretion machine: yet another player for protein transport across membranes. Microbiology 154: 1570-1583.
7. Pukatzki S, McAuley S B, Miyata S T (2009) The type VI secretion system: translocation of effectors and effector-domains. Curr Opin Microbiol 12: 11-17.
8. Ma A T, McAuley S, Pukatzki S, Mekalanos J J (2009) Translocation of a Vibrio cholerae type VI secretion effector requires bacterial endocytosis by host cells. Cell Host Microbe 5: 234-243.
9. Suarez G, Sierra J C, Erova T E, Sha J, Horneman A J, et al. (2009) A type VI secretion system effector protein VgrG1 from Aeromonas hydrophila that induces host cell toxicity by ADP-ribosylation of actin. J. Bacteriol.
10. Hood R D, Singh P, Hsu F, Guvener T, Carl M A, et al. (2010) A type VI secretion system of Pseudomonas aeruginosa targets a toxin to bacteria. Cell Host Microbe 7: 25-37.
11. Mougous J D, Gifford C A, Ramsdell T L, Mekalanos J J (2007) Threonine phosphorylation post-translationally regulates protein secretion in Pseudomonas aeruginosa. Nat Cell Biol 9: 797-803.
12. Aschtgen M S, Gavioli M, Dessen A, Lloubes R, Cascales E (2010) The SciZ protein anchors the enteroaggregative Escherichia coli Type VI secretion system to the cell wall. Mol. Microbiol.
13. Zheng J, Leung K Y (2007) Dissection of a type VI secretion system in Edwardsiella tarda. Mol Microbiol 66: 1192-1206.
14. Mougous J D, Cuff M E, Raunser S, Shen A, Zhou M, et al. (2006) A virulence locus of Pseudomonas aeruginosa encodes a protein secretion apparatus. Science 312: 1526-1530.
15. Bonemann G, Pietrosiuk A, Diemand A, Zentgraf H, Mogk A (2009) Remodelling of VipA/VipB tubules by ClpV-mediated threading is crucial for type VI protein secretion. Embo J 28: 315-325.
16. Hsu F, Schwarz S, Mougous JD (2009) TagR promotes PpkA-catalysed type VI secretion activation in Pseudomonas aeruginosa. Mol Microbiol 72: 1111-1125.
17. Pukatzki S, Ma A T, Revel A T, Sturtevant D, Mekalanos J J (2007) Type VI secretion system translocates a phage tail spike-like protein into target cells where it cross-links actin. Proc Natl Acad Sci USA 104: 15508-15513.
18. Kanamaru S (2009) Structural similarity of tailed phages and pathogenic bacterial secretion systems. Proc Natl Acad Sci USA 106: 4067-4068.
19. Leiman P G, Basler M, Ramagopal U A, Bonanno J B, Sauder J M, et al. (2009) Type VI secretion apparatus and phage tail-associated protein complexes share a common evolutionary origin. Proc Natl Acad Sci USA 106: 4154-4159.
20. Pell L G, Kanelis V, Donaldson L W, Howell P L, Davidson A R (2009) The phage lambda major tail protein structure reveals a common evolution for long-tailed phages and the type VI bacterial secretion system. Proc Natl Acad Sci USA 106: 4160-4165.
21. Wiersinga W J, van der Poll T, White N J, Day N P, Peacock S J (2006) Melioidosis: insights into the pathogenicity of Burkholderia pseudomallei. Nat Rev Microbiol 4: 272-282.
22. Brett P J, DeShazer D, Woods D E (1998) Burkholderia thailandensis sp. nov., a Burkholderia pseudomallei-like species. Int J Syst Bacteriol 48 Pt 1: 317-320.
23. Kim H S, Schell M A, Yu Y, Ulrich R L, Sarria S H, et al. (2005) Bacterial genome adaptation to niches: divergence of the potential virulence genes in three Burkholderia species of different survival strategies. BMC Genomics 6: 174.
24. Yu Y, Kim H S, Chua H H, Lin C H, Sim S H, et al. (2006) Genomic patterns of pathogen evolution revealed by comparison of Burkholderia pseudomallei, the causative agent of melioidosis, to avirulent Burkholderia thailandensis. BMC Microbiol 6: 46.
25. Nierman W C, DeShazer D, Kim H S, Tettelin H, Nelson K E, et al. (2004) Structural flexibility in the Burkholderia mallei genome. Proc Natl Acad Sci USA 101: 14246-14251.
26. Haraga A, West T E, Brittnacher M J, Skerrett S J, Miller S I (2008) Burkholderia thailandensis as a model system for the study of the virulence-associated type III secretion system of Burkholderia pseudomallei. Infect Immun 76: 5402-5411.
27. Schell M A, Ulrich R L, Ribot W J, Brueggemann E E, Hines H B, et al. (2007) Type VI secretion is a major virulence determinant in Burkholderia mallei. Mol Microbiol 64: 1466-1485.
28. Shalom G, Shaw J G, Thomas M S (2007) In vivo expression technology identifies a type VI secretion system locus in Burkholderia pseudomallei that is induced upon invasion of macrophages. Microbiology 153: 2689-2699.
29. Pilatz S, Breitbach K, Hein N, Fehlhaber B, Schulze J, et al. (2006) Identification of Burkholderia pseudomallei genes required for the intracellular life cycle and in vivo virulence. Infect Immun 74: 3576-3586.
30. West T E, Frevert C W, Liggitt H D, Skerrett S J (2008) Inhalation of Burkholderia thailandensis results in lethal necrotizing pneumonia in mice: a surrogate model for pneumonic melioidosis. Trans R Soc Trop Med Hyg 102 Suppl 1: S119-126.
31. Sun G W, Chen Y, Liu Y, Tan G Y, Ong C, et al. (2010) Identification of a regulatory cascade controlling Type III Secretion System 3 gene expression in Burkholderia pseudomallei. Mol. Microbiol.
32. Janssens S, Beyaert R (2002) A universal role for MyD88 in TLR/IL-1R-mediated signaling. Trends Biochem Sci 27: 474-482.
33. West T E, Hawn T R, Skerrett S J (2009) Toll-like receptor signaling in airborne Burkholderia thailandensis infection. Infect Immun.
34. Choi K H, Schweizer H P (2006) mini-Tn7 insertion in bacteria with single attTn7 sites: example Pseudomonas aeruginosa. Nat Protoc 1: 153-161.
35. He S Y, Nomura K, Whittam T S (2004) Type III protein secretion mechanism in mammalian and plant pathogens. Biochim Biophys Acta 1694: 181-206.
36. Christie P J, Vogel JP (2000) Bacterial type IV secretion: conjugation systems adapted to deliver effector molecules to host cells. Trends Microbiol 8: 354-360.
37. Burtnick M N, DeShazer D, Nair V, Gherardini F C, Brett P J (2010) Burkholderia mallei cluster 1 type VI secretion mutants exhibit growth and actin polymerization defects in RAW 264.7 murine macrophages. Infect Immun 78: 88-99.
38. Chobchuenchom W, Bhumiratana A (2003) Isolation and characterization of pathogens attacking Pomacea canaliculata. World Journal of Microbiology and Biotechnology 19: 903-906.
39. Chobchuenchom W, Mongkolsuk S, Bhumiratana A (1996) Biodegradation of 3-chlorobenzoate by Pseudomonas putida 10.2. World Journal of Microbiology and Biotechnology 12: 607-614.
40. Gjermansen M, Nilsson M, Yang L, Tolker-Nielsen T (2009) Characterization of starvation-induced dispersion in Pseudomonas putida biofilms: genetic elements and molecular mechanisms. Mol. Microbiol.
41. Tolker-Nielsen T, Brinch U C, Ragas P C, Andersen J B, Jacobsen C S, et al. (2000) Development and dynamics of Pseudomonas sp. biofilms. J Bacteriol 182: 6482-6489.
42. Hinsa S M, O'Toole G A (2006) Biofilm formation by Pseudomonas fluorescens WCS365: a role for LapD. Microbiology 152: 1375-1383.
43. Compant S, Duffy B, Nowak J, Clement C, Barka E A (2005) Use of plant growth-promoting bacteria for biocontrol of plant diseases: principles, mechanisms of action, and future prospects. Appl Environ Microbiol 71: 4951-4959.
44. Blango M G, Mulvey M A (2009) Bacterial landlines: contact-dependent signaling in bacterial populations. Curr Opin Microbiol 12: 177-181.
45. Chandler J R, Duerkop B A, Hinz A, West T E, Herman J P, et al. (2009) Mutational analysis of Burkholderia thailandensis quorum sensing and self-aggregation. J Bacteriol 191: 5901-5909.
46. Horton R M, Ho S N, Pullen J K, Hunt H D, Cai Z, et al. (1993) Gene splicing by overlap extension. Methods Enzymol 217: 270-279.
47. Choi K H, Mima T, Casart Y, Rholl D, Kumar A, et al. (2008) Genetic tools for select-agent-compliant manipulation of Burkholderia pseudomallei. Appl Environ Microbiol 74: 1064-1075.
48. Choi K H, Gaynor J B, White K G, Lopez C, Bosio C M, et al. (2005) A Tn7-based broad-range bacterial cloning and expression system. Nat Methods 2: 443-448.
49. Lambertsen L, Sternberg C, Molin S (2004) Mini-Tn7 transposons for site-specific tagging of bacteria with fluorescent proteins. Environ Microbiol 6: 726-732.
50. Adachi O, Kawai T, Takeda K, Matsumoto M, Tsutsui H, et al. (1998) Targeted disruption of the MyD88 gene results in loss of IL-1- and IL-18-mediated function. Immunity 9: 143-150.
51. Sternberg C, Tolker-Nielsen T (2006) Growing and analyzing biofilms in flow cells. Curr Protoc Microbiol Chapter 1: Unit 1B 2.
52. Sterk P, Kersey P J, Apweiler R (2006) Genome Reviews: standardizing content and representation of information about complete genomes. Omics 10: 114-118.
53. Altschul S F, Madden T L, Schaffer A A, Zhang J, Zhang Z, et al. (1997) Gapped BLAST and PSI-BLAST: a new generation of protein database search programs. Nucleic Acids Res 25: 3389-3402.
54. Marchler-Bauer A, Anderson J B, Chitsaz F, Derbyshire M K, DeWeese-Scott C, et al. (2009) CDD: specific functional annotation with the Conserved Domain Database. Nucleic Acids Res 37: D205-210.
55. Edgar R C (2004) MUSCLE: multiple sequence alignment with high accuracy and high throughput. Nucleic Acids Res 32: 1792-1797.
56. Gascuel O (1997) BIONJ: an improved version of the NJ algorithm based on a simple model of sequence data. Mol Biol Evol 14: 685-695.

### Example 4

### Heterologous Expression of the HSI-1-Encoded T6SS of P. Aeruginosa in E. coli

A fosmid (commercial fosmid—pCC2FOS) containing a large fragment of the P. aeruginosa chromosome containing PA0066-PA0094 (PA0095 present, but truncated), which includes all known essential structure genes of the H1-T6SS, was transferred into E. coli SW102 for recombineering (see http://web.ncifcrf.gov/research/brb/recombineeringInformation.aspx). To obtain inducible expression of the system, the native outward facing promoters of HSI-I (region between PA0082 and PA0082) were replaced with T7 promoters. This modified plasmid was placed in E. coli BL21 DE3, wherein T7 polymerase is expression can be induced by IPTG. Based on Western blot experiments demonstrating the presence of an α-Hcp1 (PA0085)-reactive band present only in induced samples bearing the HSI-I-encoding plasmid (data not shown), we concluded that successful inducible expression of secretion system components was obtained.

### Example 5

### Use of Tse1 and Tse3 as Bacteriolytic Enzymes when Delivered to the Periplasm Heterologously or Via the T6SS

### Abstract

Peptidoglycan is the major structural constituent of the bacterial cell wall, forming a meshwork outside the cytoplasmic membrane that maintains cell shape and prevents lysis. In Gram-negative bacteria, peptidoglycan is located in the periplasm, where it is protected from exogenous lytic enzymes by the outer membrane. Here we show that the type VI secretion system (T6SS) of Pseudomonas aeruginosa breaches this barrier to deliver two effector proteins, Tse1 and Tse3, to the periplasm of recipient cells. In this compartment, the effectors hydrolyze peptidoglycan, thereby providing a fitness advantage for P. aeruginosa cells in competition with other bacteria. To protect itself from lysis by Tse1 and Tse3, P. aeruginosa utilizes specific periplasmically-localized immunity proteins. The requirement for these immunity proteins depends on intercellular self-intoxication through an active T6SS, indicating a mechanism for export whereby effectors do not access donor cell periplasm in transit.

### Introduction

Competition among bacteria for niches is widespread, fierce and deliberate. These organisms elaborate factors ranging in complexity from small diffusible molecules, to exported proteins, to multicomponent machines, in order to inhibit the proliferation of rival cells^{1,2}. A common target of such factors is the peptidoglycan cell wall³⁻⁶. The conserved, essential, and accessible nature of this molecule makes it an Achilles' heel of bacteria.

The T6SS is a complex and widely distributed protein export machine capable of cell contact-dependent targeting of effector proteins between Gram-negative bacterial cells⁷⁻¹⁰. However, the mechanisms by which effectors are delivered via the secretory apparatus, and the function(s) of the effectors within recipient cells, have remained elusive. Current models of the T6SS derive from the observation that several of its components share structural homology to bacteriophage proteins¹¹⁻¹³; it has been proposed that target cell recognition and effector delivery occur in a process analogous to bacteriophage entry¹⁴.

The observation that T6S can target bacteria was originally made through studies of the hemolysin co-regulated protein secretion island I (HSI-I)-encoded T6SS(H1-T6SS) of P. aeruginosa, which exports at least three proteins, Tse1-3^{7,13}. These proteins are unrelated to each other and lack significant primary sequence homology to characterized proteins. One substrate, Tse2, is toxic by an unknown mechanism in the cytoplasm of recipient cells lacking Tsi2, a Tse2-specific immunity protein. Here we show that Tse1 and Tse3 are lytic enzymes that degrade peptidoglycan via amidase and muramidase activity, respectively. Unlike related enzymes associated with other secretion systems¹⁵, these proteins are not required for the assembly of a functional secretory apparatus. Instead, Tse1 and Tse3 function as lytic antibacterial effectors that depend upon T6S to breach the barrier imposed by the Gram-negative outer membrane.

Contacting P. aeruginosa cells actively intoxicate each other with Tse1 and Tse3. However, the peptidoglycan of P. aeruginosa is not inherently resistant to the activities of these enzymes. To protect itself, the bacterium synthesizes immunity proteins—type VI secretion immunity 1 and 3 (Tsi1 and Tsi3)—that specifically interact with and inactivate cognate toxins in the periplasm. Orthologs of tsi1 and tsi3 appear restricted to P. aeruginosa, therefore the species is able to exploit the H1-T6SS to target closely related organisms that are likely to compete for overlapping niches, while minimizing the fitness cost associated with self-targeting.

### Tse1 and Tse3 are Lytic Enzymes

To identify potential functions of Tse1 and Tse3, we searched their sequences for catalytic motifs using structure prediction algorithms¹⁶. Interestingly, motifs present in peptidoglycan degrading enzymes were apparent in both proteins (FIG. 15a). Tse1 contains invariant catalytic amino acids present in cell wall amidases (DL-endopeptidases)¹⁷, whereas Tse3 possesses a motif that includes a catalytic glutamic acid found in muramidases^{18,19}.

To test our predictions, we incubated purified Tse1 and Tse3 (Supplementary FIG. 2) with isolated E. coli peptidoglycan sacculi. Soluble products released by the enzymes were separated by high performance liquid chromatography (HPLC) and analyzed by mass spectrometry (MS). To generate separable fragments, Tse1-treated samples were digested with cellosyl, a muramidase, prior to HPLC. The observed absence of the major crosslinked fragment, and the formation of two Tse1-specific products, is consistent with enzymatic cleavage of an amide bond in the peptidoglycan peptide crosslink (FIG. 15b). Moreover, our MS data suggest that the enzyme possesses specificity for the γ-D-glutamyl-L-meso-diaminopimelic acid bond in the donor peptide stem (FIG. 15c). A variant of Tse1 containing an alanine substitution in its predicted catalytic cysteine ((C30A), Tse1*) did not degrade peptidoglycan (FIG. 15b).

Soluble peptidoglycan fragments released by Tse3 confirmed our prediction that the enzyme cleaves the glycan backbone between N-acetylmuramic acid (MurNAc) and N-acetylglucosamine (GlcNAc) residues (FIG. 15d). Enzymes that cleave this bond can do so hydrolytically (lysozymes) or non-hydrolytically (lytic transglycosylases); the latter results in the formation of 1,6-anhydroMurNAc. Our analyses showed that Tse3 possesses lysozyme-like activity and furthermore suggest that its activity is limited to a fraction of the MurNAc-GlcNAc bonds. The enzyme solubilized a significant proportion of the sacculi to release non-crosslinked peptidoglycan fragments and high molecular weight, soluble peptidoglycan fragments (FIG. 15c). A Tse3 protein with glutamine substituted at the site of the predicted catalytic glutamic acid ((E250Q), Tse3*) displayed significantly diminished activity.

If Tse1 and Tse3 degrade peptidoglycan, we reasoned the enzymes might have the capacity to lyse bacterial cells. Ectopic expression of Tse1 and Tse3 in the cytoplasm of Escherichia coli resulted in no significant lysis (data not shown). However, periplasmically-localized forms of both proteins (peri-Tse1, peri-Tse3) abruptly lysed cells following induction (FIG. 15e). In accordance with our in vitro studies, peri-Tse1* and peri-Tse3* did not induce lysis at expression levels equivalent to those of the native enzymes (data not shown). We also examined cells producing the periplasmically localized enzymes using fluorescence microscopy. Consistent with our biochemical data, cells producing peri-Tse1 were amorphous or spherical, while those producing peri-Tse3 were swollen and filamentous (FIG. 15f). In total, these data demonstrate that Tse1 and Tse3 are enzymes that degrade peptidoglycan in vivo, and that, unlike related enzymes involved in cell wall metabolism, they possess no inherent means of accessing their substrate in the periplasmic space.

T6S Function does not Require Tse1&3

Since the Tse enzymes alone are unable to reach their target cellular compartment, we hypothesized that their function must be linked to export by the T6SS. In this regard, they could: 1) remodel donor peptidoglycan to allow for the assembly of the mature T6S apparatus, 2) remodel recipient cell peptidoglycan to facilitate the passage of the T6S apparatus through the recipient cell wall, or 3) act as antibacterial effectors that compromise recipient cell wall integrity. To determine if Tse1 and Tse3 are essential for T6S apparatus assembly, we examined whether the enzymes are required for export of the third effector, Tse2. The secretion of Tse2 was not diminished in a strain lacking tse1 and tse3, suggesting that assembly of the T6S apparatus is unhindered by their absence (FIG. 16a). If Tse1 and Tse3 act as enzymes that remodel recipient cell peptidoglycan to facilitate effector translocation, Tse2 action on recipient cells should be severely impaired or nullified in the Δtse1 Δtse3 background. Instead, we found that this strain retained the ability to functionally target Tse2 to recipient cells (FIG. 16b). These findings led us to further examine the hypothesis that Tse1 and Tse3 are effector proteins rather than accessory enzymes of the T6S apparatus.

### Immunity Proteins Inhibit Tse1&3

Previous data indicate that P. aeruginosa can target itself via the T6SS⁷. If Tse1 and Tse3 act as antibacterial effectors, it follows that P. aeruginosa must be immune to their toxic effects. The tse1 and tse3 genes are each found in predicted bicistronic operons with a hypothetical gene, henceforth referred to as tsi1 and tsi3, respectively. Immunity proteins often inactivate their cognate toxin by direct interaction²⁰; therefore, as a first step toward defining a functional link between cognate Tsi and Tse proteins, we asked whether they physically associate. A solution containing a mixture of purified Tse1 and Tse3 was mixed with E. coli lysates containing either Tsi1 or Tsi3. Co-immunoprecipitation studies indicated that Tsi1 and Tsi3 interact specifically with Tse1 and Tse3, respectively, and interactions between non-cognate pairs were not detected (FIG. 17a). To investigate the immunity properties of the Tsi proteins, we measured their ability to inhibit toxicity of peri-Tse1 and peri-Tse3 in E. coli. Both Tsi1 and Tsi3 significantly decreased the toxicity of cognate, but not non-cognate Tse proteins (FIG. 17b). These results show that the activity of periplasmic Tse1 and Tse3 is specifically inhibited by cognate Tsi proteins.

### T6S Delivers Tse1&3 to the Periplasm

Most genes encoding immunity functions are essential in the presence of their cognate toxins. However, mutations that inactivate tsi1 and tsi3 are readily generated in P. aeruginosa strains that constitutively express and export Tse1 and Tse3. Based on this observation, we hypothesized that under standard laboratory conditions, the Tse proteins do not efficiently access their substrate in the periplasm. This suggests that T6S occurs by a mechanism wherein effectors are denied access to donor cell periplasm and are instead released directly to the periplasm of the recipient cell. According to this mechanism, the tsi genes would only be essential when a strain is grown under conditions that permit intercellular transfer of effectors between neighboring cells by the T6SS. As predicted, deletions in tsi1 and tsi3 severely impaired the growth of P. aeruginosa on a solid substrate, a condition conducive to T6S-based effector delivery (FIG. 17c)^{21,22}. In contrast, this growth inhibition did not occur in liquid media, which is not conducive to effector delivery by the T6SS (FIG. 17d). The growth inhibition phenotype required a functional T6SS and intact cognate effector genes, and consistent with the proposed functions of Tse1 and Tse3 in compromising cell wall integrity, growth of immunity deficient strains was fully rescued by increasing the osmolarity of the medium (FIG. 17c).

Bioinformatic analyses suggested that the Tsi proteins reside in the periplasm—Tsi1 as a soluble periplasmic protein and Tsi3 as an outer membrane lipoprotein. These predictions were confirmed by subcellular fractionation experiments, which indicated enrichment of the proteins in the periplasmic compartment (FIG. 18a). This result, taken together with the observation that the Tsi proteins interact directly with their cognate Tse proteins (FIG. 17a), provided us with a means of addressing whether the T6SS delivers Tse proteins intercellularly to the periplasm. We reasoned that if the Tse proteins are indeed delivered to the periplasm of another bacterial cell, not only should we be able to observe intoxication between distinct donor and recipient strains of P. aeruginosa, but the production of an otherwise competent immunity protein that is mislocalized to the cytoplasm should not be able to prevent such intoxication.

In growth competition assays between distinct donor and recipient strains of P. aeruginosa, we found that recipient cells that lack Tse3 immunity and are incapable of self-intoxication (Δtse3 Δtsi3), display a growth disadvantage against donor bacteria. This phenotype depends on H1-T6SS function and Tse3 in the donor strain. In the recipient strain, ectopic expression of wild-type tsi3, but not an allele encoding a signal sequence-deficient protein (Tsi3—SS), rescues the fitness defect (FIG. 18b). Importantly, the Tsi3—SS protein used in this experiment does not reach the periplasm, and retains activity in vitro as judged by interaction with Tse3 (FIG. 18a). The Tsi3—SS protein also fails to rescue the intercellular self-intoxication growth phenotype of Δtsi3 (data not shown). Analogous experiments with Tsi1 were not feasible, as the protein was unstable in the cytoplasm.

The most parsimonious explanation for T6S-mediated intercellular toxicity by Tse1 and Tse3 is that the apparatus provides a conduit for the effectors through the outer membrane of recipient cells. This led us to predict that exogenous Tse1 and Tse3 would not lyse intact P. aeruginosa. Furthermore, we posited that if the outer membrane was the relevant barrier to Tse1 and Tse3 toxicity, compromising its integrity should render P. aeruginosa susceptible to exogenous administration of the enzymes.

To test these predictions, we measured lysis of permeabilized and intact P. aeruginosa following addition of exogenous Tse1. We did not test Tse3, as the filamentous phenotype induced by this enzyme would not affect non-growing, permeabilized cells. Intact P. aeruginosa cells were not affected by the addition of exogenous Tse1; conversely, permeabilized P. aeruginosa was highly susceptible to lysis by the enzyme (FIG. 18c). Lysis induced by Tse1 is linked to its enzymatic function, as Tse 1* failed to significantly lyse cells. In total, our data show that the T6SS breaches the outer membrane to deliver lytic effector proteins directly to recipient cell periplasm.

To determine whether the T6SS can target the Tse proteins to cells of another Gram-negative organism, we conducted growth competition assays between P. aeruginosa and P. putida. These bacteria can be co-isolated from the environment²³ and are likely to compete for niches²⁴. While inactivation of either tse1 or tse3 only modestly affected the outcome of P. aeruginosa-P. putida competition assays, the fitness of P. aeruginosa lacking both genes or a functional T6SS was dramatically impaired (FIG. 18a). This partial redundancy is congruent with the enzymes exerting their effects through a single target—peptidoglycan—in the recipient cell. The fitness advantage provided by Tse1 and Tse3 was lost in liquid medium, consistent with cell contact-dependent delivery of the proteins to competitor cells (FIG. 18d). These data indicate that the T6SS targets its effectors to other species of bacteria and that these proteins can be key determinants in the outcome of interspecies bacterial interactions. In contrast with intraspecies intoxication, interspecies intoxication via the T6SS does not require the inactivation of a negative regulator of the system (eg. ΔretS), suggesting that T6S function is stimulated in response to rival bacteria.

### Discussion

Our data lead us to propose a model for T6S-catalyzed translocation of effectors to the periplasm of recipient bacteria (FIG. 19). This model provides a mechanistic framework for understanding the form and function of this complex secretion system. Our findings strengthen the existing hypothesis that the T6SS is evolutionarily and functionally related to bacteriophage^{8,14,25}. Neither the T4 bacteriophage tail spike nor other components of the puncturing device are thought to cross the inner membrane; instead, bacteriophage DNA is released to the periplasm and subsequently enters the cytoplasmic compartment using another pathway²⁶. By analogy, the Tse proteins would utilize T6S components as a puncturing device to gain access to the periplasm, whereupon Tse2 may then utilize an independent route to access the cytoplasm (FIG. 19).

Niche competition in natural environments has clearly selected for potent antibacterial processes; however, the human body is also home to a complex and competitive microbiota^{27,28}. Commensal bacteria form a protective barrier, and the ability of pathogens to colonize the host is not only dependent upon suppression or subversion of host immunity, but also can depend on their ability to displace these more innocuous organisms²⁹⁻³¹. In polymicrobial infections, Gram-negative bacteria, including P. aeruginosa, often viewith other Gram-negative bacteria for access to nutrient-rich host tissue³². Factors such as the T6SS, that influence the relative fitness of these organisms, are thus likely to impact disease outcome.

### Methods Summary

P. aeruginosa strains used in this study were derived from the sequenced strain PAO1³³. All deletions were in-frame and unmarked, and were generated by allelic exchange. E. coli growth curves were conducted using BL21 pLysS cells harboring expression plasmids for tse and tsi genes. Intercellular self-intoxication and interbacterial competition assays were performed by spotting mixed overnight cultures on a nitrocellulose membrane placed on a 3% agar growth medium. Samples were incubated at 37° C. (P. aeruginosa-P. aeruginosa) or 30° C. (P. aeruginosa-P. putida) for 12 or 24 hours. Tse1-catalyzed P. aeruginosa lysis was measured by placing cells in a minimal buffer ±1.5 mM EDTA containing either Tse1, Tse1* or lysozyme. The change in optical density at 600 nm following 5 min of incubation was used to calculate lysis. For determination of Tse1 and Tse3 activity, isolated E. coli peptidoglycan sacculi were incubated with the purified enzymes (100 μg/mL). The resulting peptidoglycan and soluble fragments released by the enzymes were separated by HPLC and their identities were determined using MS as described previously³⁴.

### Methods

Bacterial Strains, Plasmids, and Growth Conditions.

P. aeruginosa strains used in this study were derived from the sequenced strain PAO1³³. P. aeruginosa strains were grown on either Luria-Bertani media (LB), or the equivalent lacking additional NaCl (LB low salt (LB-LS): 10 g bactopeptone and 5 g yeast extract per liter) at 37° C. supplemented with 30 μg ml⁻¹ gentamycin, 25 μg ml⁻¹ irgasan, 5% w/v sucrose, 40 μg/ml X-gal, and stated concentrations of IPTG as required. E. coli strains included in this study included DH5α for plasmid maintenance, SM10 for conjugal transfer of plasmids into P. aeruginosa, BL21 pLysS for expression of Tse1 and Tse3 for toxicity and lysis, and Shuffle® T7 pLysS Express (New England Biolabs), for purification of Tse1 and Tse3. All E. coli strains were grown on either LB or LB-LS at 37° C. supplemented with 15 μg ml⁻¹ gentamycin, 150 μg ml⁻¹ carbenicillin, 50 μg ml⁻¹ kanamycin, 30 μg ml⁻¹ chloramphenicol, 200 μg ml⁻¹ trimethoprim, 0.1% rhamnose, and stated concentrations of IPTG as required. P. putida used in this study was the sequenced strain, KT2440²⁴. P. putida was grown on LB or LB-LS at 30° C. In all experiments where expression from a plasmid was required, strains were grown on media supplemented with required antibiotics to select for plasmid maintenance.

Plasmids used for inducible expression were pPSV35CV for P. aeruginosa and pET29b+ (Novagen), pET22b+ (Novagen), pSCrhaB2³⁸ and pPSV35CV for E. coli³⁹. Chromosomal deletions were made as described previously⁴⁰.

DNA Manipulations.

The creation, maintenance, and transformation of plasmid constructs followed standard molecular cloning procedures. All primers used in this study were obtained from Integrated DNA Technologies. DNA amplification was carried out using either Phusion® (New England Biolabs) or Mangomix™ (Bioline). DNA sequencing was performed by Genewiz® Incorporated. Restriction enzymes were obtained from New England Biolabs. SOE PCR was performed as previously described⁴¹.

Plasmid Construction.

pPSV35CV, pEXG2, and pSCrhaB2 have been described previously³⁸⁻⁴⁰. E. coli pET29+ expression vectors for Tse1 and Tse3 were constructed by standard cloning techniques following amplification from PAO1 chromosomal DNA using the primer pairs 1289/1290 and 1291/1292, respectively. E. coli pET22b+ expression vectors for Tse1 and Tse3 were constructed in a similar manner using primer pairs 1477/1478 and 1475/1476. Point mutations were introduced using Quikchange (Stratagene) with primer pairs 1479/1480 and 1481/1482 for the production of tse/(C30A) and tse3(E250Q), respectively.

pPSV35CV expression vectors for Tsi1 and Tsi3 were generated by amplifying the genes from genomic DNA using primer pairs 1469/1470 and 1472/1473, respectively. The Tsi3—SS pPSV35CV expression vector was generated from a product amplified using the primer pair 1522/1473. The pSCrhaB2 vectors for expressing Tsi proteins in E. coli were produced by amplifying the genes using primer pairs 1470/1497 for tsi1 and 1473/1498 for tsi3. A VSV-epitope tag was then cloned downstream of these two genes for the purpose of tagged-expression.

All deletions were in-frame and were generated by exchange with deletion alleles constructed by SOE PCR. For tse1, tse3, tsi1, and tsi3 deletion constructs, upstream DNA flanking sequences were amplified by 628/629, 735/736, 721/722, and 1485/1486, respectively. Downstream flanking DNA sequences were amplified by 630/631, 737/738, 723/724, and 1487/1488, respectively. Deletions of both effector and immunity protein were accomplished by amplifying upstream regions of tse1-tsi1 and tse3-tsi3 with 721/722 and 735/736 respectively and downstream regions with 628/629 and 835/836 respectively.

Growth Curves.

For E. coli growth curves BL21 pLysS cells harboring expression plasmids were grown overnight in liquid LB shaking at 37° C. and subinoculated to a starting optical density at 600 nm (OD₆₀₀) of between 0.01 and 0.02 in LB-LS. Cultures were grown to OD₆₀₀ 0.1-0.2 and induced with 0.1 mM IPTG. The vector pET29b+ was used for expression of native Tse1 and Tse3, and the pET22b+ vector was used for expression of periplasmic Tse1 and Tse3, and catalytic amino acid substitutions thereof. Both vectors added a C-terminal hexahistidine tag to expressed proteins, allowing for western blot analysis of expression. Samples for western blot analysis were taken 30 minutes after induction for Tse1, peri-Tse1, and peri-Tse1* and 45 minutes after induction for Tse3, peri-Tse3, and peri-Tse3*.

For P. aeruginosa growth curves, cells were grown overnight at 37° C. in liquid LB with shaking and sub-inoculated 1:1000 into LB-LS. Growth was measured by enumerating c.f.u. from plate counts of samples taken at the indicated time points.

E. coli Toxicity Measurements.

Overnight LB cultures of E. coli harboring pET22b+ expression vectors and E. coli harboring both pET22b+ and pSCrhaB2 expression vectors were serially diluted in LB to 10⁶ as 10-fold dilutions. These dilutions were spotted onto LB-LS agar with the following concentrations of inducer molecules: 0.075 mM IPTG for pET22b⁺::tse1, pET22b+::tse3 and the associated vector control, 0.02 mM IPTG and 0.1% rhamnose for pET22b+::tse1 pSCrhaB2::tsi1 and all associated controls, and 0.05 mM IPTG and 0.1% rhamnose for pET22b⁺::tse3 pSCRhaB2::tsi3 and all associated controls. Pictures were taken between 20 and 26 hours after plating.

Subcellular Fractionation.

P. aeruginosa ΔretS cells harboring expression vectors for Tsi1-V, Tsi3-V, or Tsi3-SS-V and an additional vector expressing TEM-1 (pPSV18) were grown overnight. This overnight culture was sub-inoculated into LB supplemented with 0.1 mM IPTG and grown to late logarithmic phase. Periplasmic and cytoplasmic fractions were prepared as described^{37,42}.

E. coli BL21 cells harboring expression vectors for Tse1*, Tse3*, peri-Tse1*, and peri-Tse3* were grown overnight and sub-inoculated into LB. For Tse1* and Tse3* fractionation cells also carried an empty pET22b vector to provide expression of TEM-1. Cells were grown to an OD₆₀₀ of 0.1 and induced with either 0.1 mM IPTG (Tse1* and peri-Tse1*) or 0.5 mM IPTG (Tse3* and peri-Tse3*). Cells were then harvested and fractionated as described⁴³.

Preparation of Proteins and Western Blotting.

Cell-associated and supernatant samples were prepared as described previously³⁹. Western blotting was performed as described previously for α-VSV-G and α-RNA polymerase¹³ with the modification that α-VSV-G antibody probing was performed in 5% BSA in Tris-buffered saline containing 0.05% v/v Tween 20. The α-Tse2 polyclonal rabbit antibody was raised against the peptide YDGDVGRYLHPDKEC (SEQ ID NO: 57) (GenScript). Western blots using both this antibody and the α-β-lactamase antibody(QED Biosciences Inc.) were performed identically to those using α-VSV-G. The α-His₅ Western blots were performed using the Penta-His HRP Conjugate Kit according to manufacturer's instructions (Qiagen).

Immunoprecipitation.

BL21 pLysS cells expressing VSV—G-tagged Tsi1, Tsi3, or Tsi3-SS were pelleted and resuspended in lysis buffer (20 mM Tris-Cl pH 7.5, 50 mM KCl, 8.0% v/v glycerol, 0.1% v/v NP 40, 1.0% v/v triton, supplemented with Dnase I (Roche), lysozyme (Roche), and Sigmafast™ protease inhibitor (Sigma) according to manufacturer instructions). Cells were disrupted by sonication to release VSV-G-tagged Tsi proteins into solution. To this suspension, Tse1 and Tse3 were added to concentrations of 30 μg ml⁻¹ and 25 μg ml⁻¹, respectively. This mixture was clarified by centrifugation, and a sample of the supernatant was taken as a pre-immunoprecipitation sample. The remainder of the supernatant was incubated with 100 μL α-VSV-G agarose beads (Sigma) for 2 hr at 4° C. Beads were washed three times with IP-wash buffer (100 mM NaCl, 25 mM KCl, 0.1% v/v triton, 0.1% v/v NP-40, 20 mM Tris-Cl pH 7.5, and 2% v/v glycerol). Proteins were removed from beads with SDS loading buffer (125 mM Tris, pH 6.8, 2% (w/v) 2-Mercaptoethanol, 20% (v/v) Glycerol, 0.001% (w/v) Bromophenol Blue and 4% (w/v) SDS) and analyzed by Western blot.

Interbacterial Competition Assays.

The inter-P. aeruginosa competitions were performed as described previously with minor modifications⁷. For experiments described in both FIG. 2b and FIG. 4b, competition assays were performed on nitrocellulose on LB or LB-LS 3% agar, respectively. Plate counts were taken of the initial inoculum to ensure a starting c.f.u. ratio of 1:1, and again after either 24 hours (FIG. 2b) or 12 hours (FIG. 4b) to obtain a final c.f.u. ratio. Donor and recipient colonies were disambiguated through fluorescence imaging (FIG. 2e) or through the activity of a β-galactosidase reporter as visualized on plates containing 40 μg/ml X-gal (FIG. 4b)⁵. Data were analyzed using a two-tailed Student's T-Test.

For interspecies competition assays, overnight cultures of P. aeruginosa and P. putida were grown overnight in LB broth at 37° C. and 30° C., respectively. Cultures were then washed in LB and resuspended to an OD₆₀₀ of 4.0 for P. aeruginosa and 4.5 for P. putida. P. putida and P. aeruginosa were mixed in a one-to-one ratio by volume, this mixture was spotted on a nitrocellulose membrane placed on LB-LS 3% agar, and the c.f.u. ratio of the organisms was measured by plate counts. The assays were incubated for 24 hours at 30° C., after which the cells were resuspended in LB broth and the final c.f.u. ratio determined through plate counts. Data were analyzed using a one-tailed Student's T-Test.

Purification of Tse1 and Tse3.

For purification, Tse1, Tse3, Tse1*, and Tse3* were expressed in pET29b+vectors in Shuffle® Express T7 lysY cells (New England Biolabs). The proteins were purified to homogeneity using previously reported methods⁴⁴, except that in all steps no reducing agents or lysozyme were used.

Bioinformatic Analyses.

Predicted structural homology was queried using PHYRE¹⁶. Alignments were performed using T-Espresso⁴⁵. Sequences of cell wall amidases and muramidases for alignments were obtained from seed sequences from PFAM⁴⁶. Critical motifs were defined by previous work in the study of NlpC/P60 and lytic transglycosylase/GEWL enzymes^{17,18}.

Enzymatic Assays.

Tse1 and Tse1*: Purified peptidoglycan sacculi (300 μg) from E. coli MC1061⁴⁷ were incubated with Tse1 or Tse1* (100 μg/ml) in 300 μl of 20 mM Tris/HCl, pH 8.0 for 4 h at 37° C. A sample with enzyme buffer instead of Tse1 served as a control. The pH was adjusted to 4.8 and the sample was incubated with 40 μg/ml of the muramidase cellosyl (kindly provided by Hochst AG, Frankfurt, Germany) for 16 h at 37° C. to convert the residual peptidoglycan or solubilized fragments into muropeptides. The sample was boiled for 10 min and insoluble material was removed by brief centrifugation. The reduced muropeptides were reduced with sodium borohydride and analysed by HPLC as described⁴⁷. Fractions 1 and 2 were collected, concentrated in a SpeedVac, acidified by 1% trifluoroacetic acid and analysed by offline electrospray mass spectrometry on a Finnigan LTQ-FT mass spectrometer (ThermoElectron, Bremen, Germany) as described³⁴.

Tse3 and Tse3*. Purified peptidoglycan sacculi (300 μg) from E. coli MC1061 were incubated with Tse3 or Tse3* (100 μg/ml) in 300 μl of 20 mM sodium phosphate, pH 4.8 for 20 h at 37° C. A sample with enzyme buffer instead of Tse3 served as a control. The samples were boiled for 10 min and centrifuged for 15 min (16,000×g). The supernatant was reduced with sodium borohydride and analysed by HPLC as described above (supernatant samples). The pellet was resuspended in 20 mM sodium phosphate, pH 4.8 and incubated with 40 μg/ml cellosyl for 14 h at at 37° C. The samples were boiled for 10 min, cleared by brief centrifugation and analysed by HPLC as described above (pellet samples). Fractions 3, 4 and 5 were collected and analysed by mass spectrometry as described above.

Self-Intoxication Assays.

PAO1 ΔretS attTn7::gfp cells bearing the indicated gene deletions were grown overnight in LB broth at 37° C. Cells were then diluted to 10³ c.f.u./mL and 20 μL of this solution was placed on a nitrocellulose membrane placed on LB-LS 3% agar or LB 3% agar (contains 1.0% w/v NaCl). Fluorescence images were acquired following 23 hours of incubation at 37° C. For quantification and complementation, non-fluorescent strains were used and 1 mM IPTG was included for induction of all strains—except for the tsi3-complemented strain, for which no IPTG was required to achieve comparable levels of expression to the tsi3—SS-complemented strain. At 23 hours cells were resuspended in LB. Plate counts of the initial inoculum and the final suspension were used to determine growth. Data were analyzed using a one-tailed Student's T-test.

Fluorescence Microscopy.

BL21 pLysS cells harboring periplasmic-expression vectors for Tse1, Tse3, and catalytic substitution mutants were grown in conditions identical to those in the E. coli growth curve experiments. Cells were harvested 30 minutes post-induction for Tse1 experiments and one-hour post-induction for Tse3 experiments. These cells were resuspended in PBS and incubated with 0.3 μM TMA-DPH (1-(4-trimethylammoniumphenyl)-6-phenyl-1,3,5-hexatriene p-toluenesulfonate) for 10 minutes. The stained cells were placed on 1% agarose pads containing PBS for microscopic analysis. Microscopy was performed as described previously³⁹.

EDTA-Permeabilization Lysis Assay.

Assays were performed as previously described with minor modifications⁴⁸. Cells were sub-inoculated into LB broth from overnight liquid cultures and grown to late logarithmic phase. Cells were washed in 20 mM Tris-Cl pH 7.5 and Tse1, Tse1*, or lysozyme were added to a final concentration of 0.01 mg/mL. An initial OD₆₀₀ measurement was taken before EDTA pH 8.0 was added to a final concentration of 1.5 mM. Cells were incubated with shaking at 37° C. for 5 minutes and a final OD₆₀₀ reading was taken. P. aeruginosa undergoes rapid autolysis under these assay conditions, thus lysis was expressed as a percentage of lysis above a buffer-only control.

### REFERENCES FOR EXAMPLE 3

1 Hayes, C. S., Aoki, S. K. & Low, D. A. Bacterial contact-dependent delivery systems. Annual review of genetics 44, 71-90, doi:10.1146/annurev.genet.42.110807.091449 (2010).
2 Hibbing, M. E., Fuqua, C., Parsek, M. R. & Peterson, S. B. Bacterial competition: surviving and thriving in the microbial jungle. Nat Rev Microbiol 8, 15-25, doi:nrmicro2259 [pii] 10.1038/nrmicro2259 (2010).
3 Grundling, A. & Schneewind, O. Cross-linked peptidoglycan mediates lysostaphin binding to the cell wall envelope of Staphylococcus aureus. J Bacteriol 188, 2463-2472, doi:188/7/2463 [pii] 10.11283B.188.7.2463-2472.2006 (2006).
4 Vollmer, W., Pilsl, H., Hantke, K., Holtje, J. V. & Braun, V. Pesticin displays muramidase activity. J Bacteriol 179, 1580-1583 (1997).
5 Brotz, H., Bierbaum, G., Markus, A., Molitor, E. & Sahl, H. G. Mode of action of the lantibiotic mersacidin: inhibition of peptidoglycan biosynthesis via a novel mechanism? Antimicrob Agents Chemother 39, 714-719 (1995).
6 Riley, M. A. & Wertz, J. E. Bacteriocins: evolution, ecology, and application. Annual review of microbiology 56, 117-137 (2002).
7 Hood, R. D. et al. A type VI secretion system of Pseudomonas aeruginosa targets a toxin to bacteria. Cell host & microbe 7, 25-37 (2010).
8 Schwarz, S., Hood, R. D. & Mougous, J. D. What is type VI secretion doing in all those bugs? Trends Microbiol (2010).
9 Cascales, E. The type VI secretion toolkit. EMBO reports 9, 735-741 (2008).
10 Boyer, F., Fichant, G., Berthod, J., Vandenbrouck, Y. & Attree, I. Dissecting the bacterial type VI secretion system by a genome wide in silico analysis: what can be learned from available microbial genomic resources? BMC genomics 10, 104 (2009).
11 Ballister, E. R., Lai, A. H., Zuckermann, R. N., Cheng, Y. & Mougous, J. D. In Vitro Self-Assembly of Tailorable Nanotubes from a Simple Protein Building Block. Proc. Natl. Acad. Sci. U.S.A. 105, 3733-3738 (2008).
12 Leiman, P. G. et al. Type VI secretion apparatus and phage tail-associated protein complexes share a common evolutionary origin. Proc Natl Acad Sci USA 106, 4154-4159 (2009).
13 Mougous, J. D. et al. A virulence locus of Pseudomonas aeruginosa encodes a protein secretion apparatus. Science 312, 1526-1530 (2006).
14 Kanamaru, S. Structural similarity of tailed phages and pathogenic bacterial secretion systems. Proc Natl Acad Sci USA 106, 4067-4068 (2009).
15 Christie, P. J., Atmakuri, K., Krishnamoorthy, V., Jakubowski, S. & Cascales, E. Biogenesis, architecture, and function of bacterial type iv secretion systems. Annual review of microbiology 59, 451-485 (2005).
16 Kelley, L. A. & Sternberg, M. J. Protein structure prediction on the Web: a case study using the Phyre server. Nature protocols 4, 363-371, doi:nprot.2009.2 [pii] 10.1038/nprot.2009.2 (2009).
17 Anantharaman, V. & Aravind, L. Evolutionary history, structural features and biochemical diversity of the N1 μC/P60 superfamily of enzymes. Genome biology 4, R11 (2003).
18 Scheurwater, E., Reid, C. W. & Clarke, A. J. Lytic transglycosylases: bacterial space-making autolysins. Int J Biochem Cell Biol 40, 586-591, doi:S1357-2725(07)00097-0 [pii] 10.1016/j.bioce1.2007.03.018 (2008).
19 Vollmer, W., Joris, B., Charlier, P. & Foster, S. Bacterial peptidoglycan (murein) hydrolases. FEMS Microbiol Rev 32, 259-286, doi:FMR099 [pii] 10.1111/j.1574-6976.2007.00099.x (2008).
20 Gerdes, K., Christensen, S. K. & Lobner-Olesen, A. Prokaryotic toxin-antitoxin stress response loci. Nat Rev Microbiol 3, 371-382 (2005).
21 Hall-Stoodley, L., Costerton, J. W. & Stoodley, P. Bacterial biofilms: from the natural environment to infectious diseases. Nat Rev Microbiol 2, 95-108, doi:10.1038/nrmicro821 (2004).
22 Schwarz, S. et al. Burkholderia type VI secretion systems have distinct roles in eukaryotic and bacterial cell interactions. PLoS Pathog 6 (2010).
23 Mortensen, J. E., Fisher, M. C. & LiPuma, J. J. Recovery of Pseudomonas cepacia and other Pseudomonas species from the environment. Infect Control Hosp Epidemiol 16, 30-32 (1995).
24 Nelson, K. E. et al. Complete genome sequence and comparative analysis of the metabolically versatile Pseudomonas putida KT2440. Environmental microbiology 4, 799-808, doi:366 [pii] (2002).
25 Pukatzki, S., Ma, A. T., Revel, A. T., Sturtevant, D. & Mekalanos, J. J. Type VI secretion system translocates a phage tail spike-like protein into target cells where it cross-links actin. Proc Natl Acad Sci USA 104, 15508-15513 (2007).
26 Rakhuba, D. V., Kolomiets, E. I., Dey, E. S. & Novik, G. I. Bacteriophage receptors, mechanisms of phage adsorption and penetration into host cell. Pol J Microbiol 59, 145-155 (2010).
27 Nelson, K. E. et al. A catalog of reference genomes from the human microbiome. Science 328, 994-999, doi:328/5981/994 [pii] 10.1126/science.1183605 (2010).
28 Qin, J. et al. A human gut microbial gene catalogue established by metagenomic sequencing. Nature 464, 59-65, doi:nature08821 [pii] 10.1038/nature08821 (2010).
29 Brook, I. Bacterial interference. Critical reviews in microbiology 25, 155-172 (1999).
30 Iwase, T. et al. Staphylococcus epidermidis Esp inhibits Staphylococcus aureus biofilm formation and nasal colonization. Nature 465, 346-349 (2010).
31 Reid, G., Howard, J. & Gan, B. S. Can bacterial interference prevent infection? Trends Microbiol 9, 424-428 (2001).
32 Gjodsbol, K. et al. Multiple bacterial species reside in chronic wounds: a longitudinal study. International wound journal 3, 225-231 (2006).
33 Stover, C. K. et al. Complete genome sequence of Pseudomonas aeruginosa PA01, an opportunistic pathogen. Nature 406, 959-964 (2000).
34 Bui, N. K. et al. The peptidoglycan sacculus of Myxococcus xanthus has unusual structural features and is degraded during glycerol-induced myxospore development. J Bacteriol 191, 494-505, doi:JB.00608-08 [pii] 10.11283B.00608-08 (2009).
35 Lei, S. P., Lin, H. C., Wang, S. S., Callaway, J. & Wilcox, G. Characterization of the Erwinia carotovora pelB gene and its product pectate lyase. J Bacteriol 169, 4379-4383 (1987).
36 Goodman, A. L. et al. A signaling network reciprocally regulates genes associated with acute infection and chronic persistence in Pseudomonas aeruginosa. Dev Cell 7, 745-754 (2004).
37 Imperi, F. et al. Analysis of the periplasmic proteome of Pseudomonas aeruginosa, a metabolically versatile opportunistic pathogen. Proteomics 9, 1901-1915, doi:10.1002/pmic.200800618 (2009).
38 Cardona, S. T. & Valvano, M. A. An expression vector containing a rhamnose-inducible promoter provides tightly regulated gene expression in Burkholderia cenocepacia. Plasmid 54, 219-228 (2005).
39 Hsu, F., Schwarz, S. & Mougous, J. D. TagR promotes PpkA-catalysed type VI secretion activation in Pseudomonas aeruginosa. Mol Microbiol 72, 1111-1125 (2009).
40 Rietsch, A., Vallet-Gely, I., Dove, S. L. & Mekalanos, J. J. ExsE, a secreted regulator of type III secretion genes in Pseudomonas aeruginosa. Proc Natl Acad Sci USA 102, 8006-8011 (2005).
41 Horton, R. M. et al. Gene splicing by overlap extension. Methods in enzymology 217, 270-279 (1993).
42 Wood, P. M. Periplasmic location of the terminal reductase in nitrite respiration. FEBS letters 92, 214-218, doi:0014-5793(78)80757-1 [pii] (1978).
43 Liu, J. & Walsh, C. T. Peptidyl-prolyl cis-trans-isomerase from Escherichia coli: a periplasmic homolog of cyclophilin that is not inhibited by cyclosporin A. Proc Natl Acad Sci USA 87, 4028-4032 (1990).
44 Mougous, J. D. et al. Identification, function and structure of the mycobacterial sulfotransferase that initiates sulfolipid-1 biosynthesis. Nature structural & molecular biology 11, 721-729 (2004).
45 Armougom, F. et al. Expresso: automatic incorporation of structural information in multiple sequence alignments using 3D-Coffee. Nucleic acids research 34, W604-608, doi:34/suppl 2/W604 [pii] 10.1093/nar/gk1092 (2006).
46 Finn, R. D. et al. The Pfam protein families database. Nucleic acids research 38, D211-222, doi:gkp985 [pii] 10.1093/nar/gkp985 (2010).
47 Glauner, B. Separation and quantification of muropeptides with high-performance liquid chromatography. Anal Biochem 172, 451-464 (1988).
48 Watt, S. R. & Clarke, A. J. Role of autolysins in the EDTA-induced lysis of Pseudomonas aeruginosa. FEMS Microbiol Lett 124, 113-119 (1994).

### Example 6

### Creation of Vectors with the Tse1 and/or Tse3 Gene

The plasmid containing Tse1 and/or Tse3 can be constructed by cloning the complete Tse1 and/or Tse3 gene into any appropriate vector, as is well known in the art. The techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2^{nd} Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

Appropriate vectors may be obtained from, for example, Vector Laboratories Inc.; Promega; Novagen; New England Biolabs; Clontech; Roche; Pharmacia; EpiCenter; OriGenes Technologies Inc.; Stratagene; Perkin Elmer; Pharmingen; and Invitrogen Corp., Carlsbad, Calif. Such vectors may then for example be used for cloning or subcloning nucleic acid molecules of interest. General classes of vectors of particular interest include prokaryotic and/or eukaryotic cloning vectors, Expression Vectors, fusion vectors, two-hybrid or reverse two-hybrid vectors, shuttle vectors for use in different hosts, mutagenesis vectors, transcription vectors, and the like.

Once the appropriate plasmid vector is chosen, PCR can be used to amplify the Tse1 and/or Tse3 gene by designing appropriate primers for the DNA sequence. The PCR primers can be designed with restriction sites or recombination sites to facilitate cloning into the desired vector backbone. All recombination sites, restriction sites, other death genes, promoters, and other plasmid DNA elements can be amplified by PCR using the appropriate primer pairs as is well known in the art. The embodiments described herein depict the various arrangements of these plasmid DNA elements, and creation of such plasmid vectors is well within the ability of one of ordinary skill in the art.

### Example 7

### Creation of Vectors with the Tsi1 and/or Tsi3 Gene

The plasmid containing Tsi1 and/or Tsi3 can be constructed by cloning the complete Tsi1 and/or Tsi3 gene into any appropriate vector, as is well known in the art. The techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2^{nd} Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

Appropriate vectors may be obtained from, for example, Vector Laboratories Inc.; Promega; Novagen; New England Biolabs; Clontech; Roche; Pharmacia; EpiCenter; OriGenes Technologies Inc.; Stratagene; Perkin Elmer; Pharmingen; and Invitrogen Corp., Carlsbad, Calif. Such vectors may then for example be used for cloning or subcloning nucleic acid molecules of interest. General classes of vectors of particular interest include prokaryotic and/or eukaryotic cloning vectors, Expression Vectors, fusion vectors, two-hybrid or reverse two-hybrid vectors, shuttle vectors for use in different hosts, mutagenesis vectors, transcription vectors, and the like.

Once the appropriate plasmid vector is chosen, PCR can be used to amplify the Tsi1 and/or Tsi3 gene by designing appropriate primers for the DNA sequence. The PCR primers can be designed with restriction sites or recombination sites to facilitate cloning into the desired vector backbone. All recombination sites, restriction sites, other death genes, promoters, and other plasmid DNA elements can be amplified by PCR using the appropriate primer pairs as is well known in the art. The embodiments described herein depict the various arrangements of these plasmid DNA elements, and creation of such plasmid vectors is well within the ability of one of ordinary skill in the art.

### Example 8

### Creation of Linear Vectors Resistant to Recircularization

In one example, the Tse1 and/or Tse3 or Tsi1 and/or Tsi3 vectors are linearized by HindIII, AccI, or other restriction digestion, which results in an overhang compatible with topoisomerase cloning, as is known in the art. Alternatively, the vectors can be prepared to have blunt or other customized overhangs at the ends of the linear vectors. As described in the literature, the ends of the vector can be covalently bound to topoisomerase Ito facilitate cloning, such that a desired DNA fragment can be incubated along with the modified linearized Tse1 and/or Tse3 or Tsi1 and/or Tsi3 vector, resulting in the DNA fragment entering the Tse1 and/or Tse3 or Tsi1 and/or Tsi3 vector at the site of the restriction digestion.

In another example, the linearized Tse1 and/or Tse3 or Tsi1 and/or Tsi3 vectors are treated with a dephosphorylating enzyme, such as alkaline phosphatase or an equivalent. This treatment reduces the likelihood that the Tse1 and/or Tse3 or Tsi1 and/or Tsi3 vector will recircularize without incorporating the DNA fragment of interest, and thus increases positive cloning efficiency. One of ordinary skill in the art can use any other modifications to the vectors which will result in increased efficiency of production of vectors with the DNA fragment of interest.

### Example 9

### Creation of Cell Lines Expressing Tse1 and/or Tse3

Any cell type can be used to express the vectors created herein.

Once the desired recombinant vector is created, the cells are transformed or transfected using standard techniques well known to one of ordinary skill in the art. In one example, the Tse1 and/or Tse3 gene is under the transcriptional control of an inducible promoter, such as the lac promoter, such that the Tse1 and/or Tse3 gene is not constitutively expressed in the cell line. Successful chromosomal integration can be selected for by using a second antibiotic resistance gene, such as chloramphenicol, which may or may not be found on the same plasmid containing the Tse1 and/or Tse3 gene. Any other selection markers can be used by one of ordinary skill in the art depending on the design of the research experiment.

### Example 10

### Positive Selection of Tsi1 and/or Tsi3-Containing Plasmids

To select for Tsi1 and/or Tsi3-containing plasmids, the Tsi1 and/or Tsi3 gene is included on a vector which will, when expressed, confer immunity to a cell which is expressing Tse1 and/or Tse3. The cells expressing Tse1 and/or Tse3 are created as described herein. In a cell line which is expressing Tse1 and/or Tse3 in the absence of Tsi1 and/or Tsi3, the cells will not survive. Any of the vectors containing the Tsi1 and/or Tsi3 gene described in the embodiments herein can be used for selection of positive clones containing the DNA fragment of interest.

If a Tse1 and/or Tse3-expressing cell receives the vector which expresses the Tsi1 and/or Tsi3 gene, that cell will survive, while such cells that do not express the Tsi1 and/or Tsi3 gene will not survive. In this selection example, the surviving cells will contain the plasmid with the DNA fragment of interest, along with Tsi1 and/or Tsi3. If the plasmid containing the DNA fragment of interest is absent, the cells will die and will not be selected.

In another example, the vector containing a Tsi1 and/or Tsi3 gene is used for selection of positive clones containing the DNA fragment of interest. Cells expressing the Tsi1 and/or Tsi3 gene also contain the DNA fragment of interest on the vector. In this method, the Tsi1 and/or Tsi3 gene can be used as a marker for a desired recombination or ligation event.

In another example, a vector containing a Tsi1 and/or Tsi3 gene flanked by one or more recombination sites gene is used for selection of positive clones containing the DNA fragment of interest. The DNA fragment of interest is inserted into a site on the vector, such that the fragment does not disrupt the Tsi1 and/or Tsi3 gene but is contained within the recombination sites. In another example, a topoisomerase or TA site is included within the flanking sites, but outside the Tsi1 and/or Tsi3 gene, to facilitate DNA fragment insertion. The vector containing the DNA fragment of interest is then combined with a second vector containing matching recombination sites, such that a positive recombination event will move the DNA fragment of interest and the Tsi1 and/or Tsi3 gene into the new vector, which can then be selected for survival in cells expressing Tse1 and/or Tse3, as described herein.

In another example, the vector containing the Tsi1 and/or Tsi3 gene flanked by one or more restriction sites is used for selection of positive clones containing the DNA fragment of interest. The DNA fragment of interest is inserted into a site on the vector, such that the fragment does not disrupt the Tsi1 and/or Tsi3 gene but is contained within the restriction sites. The vector containing the DNA fragment of interest and a second cloning vector are then digested with one or more restriction enzymes, followed by a ligation reaction. A positive ligation event will move the DNA fragment of interest and the Tsi1 and/or Tsi3 gene into the second cloning vector, which can then be selected for survival in cells expressing Tse1 and/or Tse3.

In one example, the vector comprising a Tsi1 and/or Tsi3 gene in an inactive form, such as a truncated form, is used for selection of positive clones containing the DNA fragment of interest. This vector can be used, for example, in methods for rescuing the activity of the Tsi1 and/or Tsi3 gene such that vectors which contain a functional Tsi1 and/or Tsi3 gene also contain the DNA fragment of interest (as described herein). The functional Tsi1 and/or Tsi3 can be rescued by recombination, integration, or other events or reactions as described herein. Vectors can be readily designed for the particular experiment by one of ordinary skill in the art.

In another example, a vector containing the Tsi1 and/or Tsi3 locus, but split into two parts on the same plasmid, is used for selection of positive clones containing the DNA fragment of interest. A fully functional Tsi1 and/or Tsi3 would assemble through homologous recombination or ligation event, such that only the cells containing a recombinant plasmid containing the DNA fragment of interest, with a functional Tsi1 and/or Tsi3 can survive transformation.

### Example 11

### Negative Selection of Tse1 and/or Tse3 Plasmids

The Tse1 and/or Tse3 recombinant vectors can be used in negative selection in order to enhance the efficiency of production of plasmids containing the desired DNA fragment of interest. In one example, the vector comprising one or more unique restriction enzyme recognition sites, wherein cloning of a nucleic acid insert into the one or more unique restriction enzyme recognition sites disrupts expression of Tse1 and/or Tse3, can be used to exclude vectors that do not contain the DNA fragment of interest. The vectors of this embodiment can be used as cloning vehicles, since cloning of an insert into the one or more restriction sites in the vector interrupts Tse1 and/or Tse3 expression and provide an easily selectable marker—cells with vectors containing no insert have their growth inhibited by Tse1 and/or Tse3 expression (so long as they do not endogenously express an antidote to Tse1 and/or Tse3), and those with inserts do not. In one preferred embodiment, one or more unique restriction sites are engineered into the coding region for Tse1 and/or Tse3 using techniques well known to those of skill in the art, such that cloning an insert into the restriction site disrupts the coding region for Tse1 and/or Tse3. In this embodiment, the restriction sites can be engineered into the coding region to result in silent nucleotide changes, or may result in one or more changes in the amino acid sequence of Tse1 and/or Tse3, so long as the encoded Tse1 and/or Tse3 protein retains cytotoxic activity. Alternatively, the one or more unique restriction sites may be located in regulatory regions such that cloning of an insert would disrupt expression of Tse1 and/or Tse3 from the vector. Design and synthesis of nucleic acid sequences and preparation of vectors comprising such sequences is well within the level of skill in the art.

The Tse1 and/or Tse3 recombinant vectors can also be used in negative selection, such as for example using the Gateway® Cloning System. Any of the vectors described in the embodiments herein can be used to exclude vectors that do not contain the DNA fragment of interest, such that a functional Tse1 and/or Tse3 gene indicates a vector which is lacking the DNA fragment of interest.

In this example, the vector containing a Tse1 and/or Tse3 gene flanked by one or more restriction enzyme sites or recombination sites can be used to exclude vectors that do not contain the DNA fragment of interest. Recombination sites include, but are not limited to, attB, attP, attL, and attR. This vector is designed such that the DNA fragment of interest (such as, for example, a PCR product) will replace the Tse1 and/or Tse3 located between the two flanking sites. If the DNA fragment of interest is present in the vector, the cells containing the vector survive, as the Tse1 and/or Tse3 gene will no longer be present on the desired recombinant vector. If the gene of interest is not present, the Tse1 and/or Tse3 gene will prevent survival of the cell carrying the undesired vector. Thus, only cells containing positive clones with the DNA fragment of interest will be viable, and easily selected for.

In another example, the vector containing a dual selection cassette, wherein the vector comprises a first gene encoding Tse1 and/or Tse3, and a second gene encoding a second selectable marker, such as an antibiotic resistance gene or a second “death” gene encoding a second toxic protein, can be used to exclude vectors that do not contain the DNA fragment of interest. The antibiotic resistance gene can be selected from either bacterial or eukaryotic genes, and can confer resistance to ampicillin, kanamycin, tetracycline, cloramphenicol, and others known in the art. The second death gene can be any suitable death gene, including but not limited to, rpsL, tetAR, pheS, thyA, lacY, gata-1, ccdB, and sacB. The second death gene can also be selected from either prokaryotic or eukaryotic toxic genes. This dual selection cassette is flanked by at least one restriction site or recombination site, such that the DNA fragment of interest will replace the dual selection cassette located between the two sites in the desired recombination or ligation event. If the DNA fragment of interest is present, the cells containing the vector survive, as the Tse1 and/or Tse3 gene will no longer be present on the desired recombinant vector. If the gene of interest is not present, the vector will still contain the Tse1 and/or Tse3 gene and will prevent survival of the cell carrying the undesired vector. This dual selection cassette can thus be used for any double negative selection strategy as desired by one of ordinary skill in the art. In one embodiment, the Tse1 and/or Tse3 gene double negative selection strategy is used when use of multiple antibiotics is not compatible with the particular selection design.

In another example, the vector containing a dual selection cassette comprising the Tse1 and/or Tse3 gene as well as a cloramphenicol resistance gene under control of at least one promoter, can be used to exclude vectors that do not contain the DNA fragment of interest. The vector is cut using restriction enzymes both upstream and downstream of the dual selection cassette. Optionally, the linearized vector can be gel purified to remove the excised dual selection cassette DNA from the reaction. DNA containing the DNA fragment of interest and appropriate restriction enzyme sites, such as a PCR product, is then combined with the linearized vector in a ligation reaction. Positive clones will be chloramphenicol sensitive and viable (Tse1 and/or Tse3 negative), due to the replacement of the dual selection cassette with the DNA fragment of interest.

In another example, the vector containing at least one recombination site within the Tse1 and/or Tse3 gene or corresponding regulatory element (e.g. promoter or enhancer), such that a desired recombination event will disrupt the expression of the Tse1 and/or Tse3 gene from the vector, can be used to exclude vectors that do not contain the DNA fragment of interest. The location of the recombination site should be chosen such that if the desired recombination event occurs, the resulting Tse1 and/or Tse3 gene will be inactive and the cell containing the desired vector will survive. If the desired recombination event does not occur, the Tse1 and/or Tse3 gene will remain intact and the cell containing the undesired vector will not survive.

In another example, the vector contains at least one restriction enzyme site within the Tse1 and/or Tse3 gene or corresponding regulatory element (e.g. promoter or enhancer), which is used to exclude vectors that do not contain the DNA fragment of interest, such that an undesired ligation event will produce an intact and functional Tse1 and/or Tse3 gene, which will result in the death of the cell containing the undesired vector.

In another example, the Tse1 and/or Tse3 gene is fragmented on multiple vectors, with shared restriction enzyme sequences or recombination site sequences connecting the gene fragments, wherein the vectors are used to exclude vectors that do not contain the DNA fragment of interest. The vectors are designed and arranged such that an undesired recombination event or ligation event will result in the creation of an intact Tse1 and/or Tse3 gene on the undesired plasmid, thus resulting in the death of the cells containing the undesired vector with the functional Tse1 and/or Tse3 gene. The vectors containing the intact Tse1 and/or Tse3 gene also are lacking the DNA fragment of interest, and are thus excluded from selection.

1. A substantially purified type VI secretion exported (Tse) protein, selected from the group consisting of Tse1, Tse2, and Tse3.

2. The substantially purified Tse protein of claim 1, wherein the Tse protein comprises a conjugate comprising the Tse protein and one or more of the following:
(a) a transduction domain;
(b) a targeting domain to carry the conjugate across a bacterial outer membrane to a periplasmic space;
(c) a phage capsid;
(d) a leader sequence.

3-5. (canceled)

6. A pharmaceutical composition comprising
(a) the substantially purified Tse protein or Tsi protein of claim 1; and
(b) a pharmaceutically acceptable carrier.

7. A host cell comprising,
(a) a plurality of genes encoding proteins capable of forming a type 6 secretion system (T6SS); and
(b) a recombinant gene encoding a therapeutic polypeptide that can be secreted by the recombinant T6SS in the recombinant cell, wherein the recombinant gene is operatively linked to a regulatory sequence.

8. A recombinant gene encoding a fusion polypeptide of
(a) a therapeutic polypeptide; and
(b) one or both of a VgrG polypeptide and a Hcp polypeptide.

9. A recombinant fusion protein comprising
(a) a therapeutic polypeptide selected from the group consisting of bactericidal proteins group HA phospholipase A2, bactericidal/permeability-increasing protein, human peptidoglycan recognition proteins 3 and 4 (PGLYRP3 and PGLYRP4), Tse1, Tse2, and Tse3; and
(b) one or both of a VgrG polypeptide and a Hcp polypeptide.

10. A pharmaceutical composition, comprising the recombinant fusion protein of claim 9 and a pharmaceutically acceptable carrier.

11. A pharmaceutical composition, comprising
(a) the host cell of claim 7; and
(b) a pharmaceutically acceptable carrier.

12. (canceled)

13. A method for inhibiting bacterial growth, comprising contacting bacteria to be inhibited with an amount of the polypeptide of claim 2 effective to inhibit bacterial growth.

14. A method for inhibiting bacterial growth, comprising contacting bacteria to be inhibited with an amount of the pharmaceutical composition of claim 11 effective to inhibit bacterial growth.

15. (canceled)

16. A method for improved biomolecule extraction from bacterial cells, comprising contacting the bacterial cells with an amount effective of Tse1 to lyse the bacterial cells during the extraction process.

17. (canceled)

18. A recombinant vector, comprising a first gene coding for type VI secretion exported protein 1 (Tse1) or, type VI secretion exported protein 1 (Tse3) wherein the first gene is operatively linked to a heterologous regulatory sequence.

19. The recombinant vector of claim 18, wherein the vector comprises one or more unique restriction enzyme recognition sites, and wherein cloning of a nucleic acid insert into the one or more unique restriction enzyme recognition sites disrupts expression of the first gene.

20. The recombinant vector of claim 18, wherein the recombinant vector comprises at least a first and a second recombination site flanking the first gene operatively linked to a regulatory sequence, wherein said first and second recombination sites do not recombine with each other.

21-23. (canceled)

24. A method for selectable cloning, comprising culturing the recombinant host cell of any one of claim 18 under conditions suitable for expression of Tse1 or Tse3 from the recombinant vector if no insert is present, and selecting those cells that grow as comprising recombinant vectors with the insert cloned into the expression vector.

25-26. (canceled)

27. The method of claim 24, wherein the recombinant host cell comprises a gene encoding Tse1, wherein the method comprises culturing the recombinant host cell under conditions suitable for expression of Tse1, and wherein the culture conditions comprise enriching for particular clones by killing off other quickly via Tse1 lytic activity.

28-31. (canceled)

32. A method for production of a cloning vector that lacks an insert, comprising culturing the recombinant host cell claim 18 under conditions suitable for vector replication and expression of Tse1 or Tse3, wherein the recombinant host cells further express a Tse1 or Tse3 antidote, and isolating vector from the host cells.

33. The method of claim 32, wherein the Tse1 or Tse3 antidote comprises Tsi1 or Tsi3.

34. A recombinant vector, comprising a nucleic acid encoding Tsi1 or Tsi3, wherein the nucleic acid is operatively linked to a regulatory sequence.

35. A host cell comprising in its genome, a first recombinant gene coding for Tse1 or Tse3 operatively linked to a regulatory sequence.

36-38. (canceled)

39. A substantially purified protein, selected from the group consisting of Tsi1, Tsi2, and Tsi3.

40. A method for improved biomolecule extraction from bacterial cells, comprising contacting the bacterial cells with an amount effective of Tse1 to lyse the bacterial cells during the extraction process.


SEQUENCE LISTING (57 sequences)

SEQ ID NO: 1 - DNA, 477 bp, Pseudomonas aeruginosa
atgtcctacgactacgagaaaaccagcctcaccctctaccgggcggtattcaaggccaactacgacggcgacgtcggtcgctacctgcatcccgacaaggaactcgccgaggctgcggaagtcgccccgctgctgcatccgaccttcgacagccccaacacccctggcgtccccgcccgcgcgccggacatcgtcgccggccgcgacggcctctacgccccggacaccggcggcacctcggtgttcgaccgcgccggcgtgctgcgccgcgccgacggcgacttcgtgatacccgacggcaccgacatcccgccggaccttaaggtgaagcaggacagctacaacaagcgcctgcaagccacccactacaccatcatgccggccaagccgatgtaccgggaggtcctcatgggccaactggacaacttcgtgcgcaacgccatccgccgccaatgggaaaaagcccgcgggctctag

SEQ ID NO: 2 - protein, 158 aa, Pseudomonas aeruginosa
MSYDYEKTSLTLYRAVFKANYDGDVGRYLHPDKELAEAAEVAPLLHPTFDSPNTPGVPARAPDIVAGRDGLYAPDTGGTSVFDRAGVLRRADGDFVIPDGTDIPPDLKVKQDSYNKRLQATHYTIMPAKPMYREVLMGQLDNFVRNAIRRQWEKARGL

SEQ ID NO: 3 - DNA, 477 bp, Pseudomonas aeruginosa
  misc_feature (21)..(21): N can be A or G
atgtcctacgactacgagaanaccagcctcaccctctaccgggcgntattcaaggccaactacganggcgangtcggtcgctacctgcnncccgacnaggaactcgccgaggcngcggaagtcgccccgctgctgcatccgaccttcgacagccccancaccccnggcgtccccgcccgcgcgccggacatcgtcgccggccgcgacggcctctacgccccggacaccggcggcacctcggtgttcgaccgcgccggcgtgctgcgccgcgccgacggcgacttcgtgatncccgacggcaccgacatcccgccggacctnaaggtgaancaggacagctacaacaagcgcctgcaagccacccactacaccatcatgccggccaagccgatgtaccgggaggtcctcatgggccanctggacaacttcgtgcgcaacgccatccgncgccaatgggaaaaagcccgcgggctctag

SEQ ID NO: 4 - protein, 246 aa, Pseudomonas aeruginosa
  MISC_FEATURE (16)..(16): X can be V or I
MSYDYEKTSLTLYRAXFKANYXGDVGRYLXPDKELAEAAEVAPLLHPTFDSPNTPGVPARAPDIVAGRDGLYAPDTGGTSVFDRAGVLRRADGDFVIPDGTDIPPDLKVKQDSYNKRLQATHYTIMPAKPMYREVLMGQLDNFVRNAIRRQWEKARGLLYAPDTGGTSVFDRAGVLRRADGDFVIPDGTDIPPDLKVKQDSYNKRLQATHYTIMPAKPMYREVLMGQLDNFVRNAIRRQWEKARGL

SEQ ID NO: 5 - protein, 149 aa, Pseudomonas aeruginosa
KTSLTLYRAVFKANYDGDVGRYLHPDKELAEAAEVAPLLHPTFDSPNTPGVPARAPDIVAGRDGLYAPDTGGTSVFDRAGVLRRADGDFVIPDGTDIPPDLKVKQDSYNKRLQATHYTIMPAKPMYREVLMGQLDNFVRNAIRRQWEKA

SEQ ID NO: 6 - protein, 239 aa, Pseudomonas aeruginosa
  MISC_FEATURE (10)..(10): X can be V or I
KTSLTLYRAXFKANYXGDVGRYLXHRPDKELAEAAEVAPLLHPTFDSPNTPGVPARAPDIVAGRDGLYAPDTGGTSVFDRAGVLRRADGDFVIPDGTDIPPDLKVKQDSYNKRLQATHYTIMPAKPMYREVLMGQLDNFVRNAIRRQWEKARGLLYAPDTGGTSVFDRAGVLRRADGDFVIPDGTDIPPDLKVKQDSYNKRLQATHYTIMPAKPMYREVLMGQLDNFVRNAIRRQWEKA

SEQ ID NO: 7 - protein, 158 aa, Pseudomonas aeruginosa
  MISC_FEATURE (9)..(9): X can be S or A
MSYDYEKTXXTLYRAXFKANYXGDVGRYLXPDKELAEAAEVAPLLHPTFDSPNTPGVPAXAPDIVAGRDGLYAPDTGGTSVFDRAGVLRRADGDFVIPDGTDIPPDLKVKQDSYNKRLXATHYTIMPAXXMYREVLMGXXDNFVRNAIXXQWEKARGL

SEQ ID NO: 8 - protein, 149 aa, Pseudomonas aeruginosa
  MISC_FEATURE (3)..(3): X can be A or S
KTXXTLYRAXFKANYXGDVGRYLXPDKELAEAAEVAPLLHPTFDSPNTPGVPAXAPDIVAGRDGLYAPDTGGTSVFDRAGVLRRADGDFVIPDGTDIPPDLKVKQDSYNKRLXATHYTIMPAXXMYREVLMGXXDNFVRNAIXXQWEKA

SEQ ID NO: 9 - DNA, 465 bp, Pseudomonas aeruginosa
atggacagtctcgatcaatgcatcgtcaacgcctgcaagaacagctgggacaagagctacctggccggcaccccgaacaaggacaactgttccggcttcgtccagtcggtggccgccgagctgggcgtaccgatgccccgcggcaacgccaacgccatggtcgacggcctggagcagagctggaccaagctcgcctccggcgccgaggccgcgcagaaggcggcccagggcttcctggtgatcgccggcctgaagggccgcacctacgggcacgtcgcggtggtcatcagcggtccgctgtatcggcagaagtacccgatgtgctggtgcggcagcatcgccggcgcggtcggccagagccagggcctgaagtcggtcggccaggtgtggaatcgcaccgaccgcgaccgcctcaactactacgtctactccctggccagttgcagcctgcccagggccagttga

SEQ ID NO: 10 - protein, 154 aa, Pseudomonas aeruginosa
MDSLDQCIVNACKNSWDKSYLAGTPNKDNCSGFVQSVAAELGVPMPRGNANAMVDGLEQSWTKLASGAEAAQKAAQGFLVIAGLKGRTYGHVAVVISGPLYRQKYPMCWCGSIAGAVGQSQGLKSVGQVWNRTDRDRLNYYVYSLASCSLPRAS

SEQ ID NO: 11 - DNA, 1227 bp, Pseudomonas aeruginosa
atgaccgccaccagcgacctgatcgagtcactgatttcctatagctgggacgactggcaggtgacccgccaggaagcccgccgggtgatcgccgcgatccgcaacgacaacgtgcccgatgcgaccatcgccgcactcgacaagagcggctcgctgatcaagctgttccagcgggtgggcccgccggaactggcgcgctcgctgatcgccagcatcgccgggcgcaccaccatgcagcgctaccaggcacgcaatgccttgatccgcagcctgatcaataatcccctgggcacccagaccgacaactggatctacttccccaccatcaccttcttcgacatctgcgccgacctcgccgacgccgctggccgcctgggcttcgccgcggccggcgccaccggggtggccagccaggcgatccagggtccgttcagcggggtcggcgccaccggcgtcaatccgaccgacctgccgtccattgccttcggcgaccagctcaagctgctcaacaaggacccggcgaccgtcaccaagtacagcaacccgctcggcgacctgggcgcctacctgagccagctttcgccgcaagacaagctgaaccaggcacagacgctggtcggccagccgatcagcacgctgttccccgacgcctatccgggcaacccgccgtcgcgggccaaggtcatgtccgcggccgcgcgcaagtacgacctgacgccgcaactgatcggcgcgatcatcctcgccgagcagcgtgaccagacccgcgacgaagacgccaaggactatcaggcggcagtcagcatcaagagcgccaacacctccatcggcctcggccaggtggtggtctccaccgcgatcaagtacgagctgttcaccgacctgctcggccagccggtgcgccgcggtctgtcgcgcaaggcggtcgccaccctgctggcttccgacgaattcaacatcttcgccaccgcccgttacatccgctacgtcgccaacctcgcgtcgcaacaggacctgcgcaagttgccgaagacccgcggcgcatttccctccatcgatctccgcgcctacgccggcaatccgcgcaactggccgcgggacaatgtccgcgcgctggcctcggaatacacctcgcggccctgggacgacaacctgtcgccgggctggccgatgttcgtcgacgatgcctacgccaccttcctcgaccctggaatgaggttcccatga

SEQ ID NO: 12 - protein, 408 aa, Pseudomonas aeruginosa
MTATSDLIESLISYSWDDWQVTRQEARRVIAAIRNDNVPDATIAALDKSGSLIKLFQRVGPPELARSLIASIAGRTTMQRYQARNALIRSLINNPLGTQTDNWIYFPTITFFDICADLADAAGRLGFAAAGATGVASQAIQGPFSGVGATGVNPTDLPSIAFGDQLKLLNKDPATVTKYSNPLGDLGAYLSQLSPQDKLNQAQTLVGQPISTLFPDAYPGNPPSRAKVMSAAARKYDLTPQLIGAIILAEQRDQTRDEDAKDYQAAVSIKSANTSIGLGQVVVSTAIKYELFTDLLGQPVRRGLSRKAVATLLASDEFNIFATARYIRYVANLASQQDLRKLPKTRGAFPSIDLRAYAGNPRNWPRDNVRALASEYTSRPWDDNLSPGWPMFVDDAYATFLDPGMRFP

SEQ ID NO: 13 - protein, 13 aa, Artificial Sequence
  Synthetic
GRKKRRQRRRPPQ

SEQ ID NO: 14 - protein, 16 aa, Artificial Sequence
  Synthetic
RQIKIWFQNRRMKWKK

SEQ ID NO: 15 - protein, 7 aa, Artificial Sequence
  Synthetic
RRMKWKK

SEQ ID NO: 16 - protein, 18 aa, Artificial Sequence
  Synthetic
RGGRLSYSRRRFSTSTGR

SEQ ID NO: 17 - protein, 10 aa, Artificial Sequence
  Synthetic
RRLSYSRRRF

SEQ ID NO: 18 - protein, 17 aa, Artificial Sequence
  Synthetic
RGGRLAYLRRRWAVLGR

SEQ ID NO: 19 - protein, 8 aa, Artificial Sequence
  Synthetic
RRRRRRRR

SEQ ID NO: 20 - protein, 11 aa, Artificial Sequence
  Synthetic
YGRKKRRQRRR

SEQ ID NO: 21 - protein, 9 aa, Artificial Sequence
  Synthetic
ILLPLLLLP

SEQ ID NO: 22 - protein, 16 aa, Artificial Sequence
  Synthetic
RQLKIWFQNRRMKWKK

SEQ ID NO: 23 - protein, 9 aa, Artificial Sequence
  Synthetic
RKKRRQRRR

SEQ ID NO: 24 - protein, 11 aa, Artificial Sequence
  Synthetic
YARAAARQARA

SEQ ID NO: 25 - protein, 12 aa, Artificial Sequence
  Synthetic
RRQRRTSKLMKR

SEQ ID NO: 26 - protein, 12 aa, Artificial Sequence
  Synthetic
AAVLLPVLLAAR

SEQ ID NO: 27 - protein, 9 aa, Artificial Sequence
  Synthetic
RRRRRRRRR

SEQ ID NO: 28 - protein, 9 aa, Artificial Sequence
  Synthetic
SGWFRRWKK

SEQ ID NO: 29 - protein, 16 aa, Artificial Sequence
  Synthetic
RQIKIWFQNRRMKWKK

SEQ ID NO: 30 - protein, 9 aa, Artificial Sequence
  Synthetic
RRRRRRRRR

SEQ ID NO: 31 - protein, 13 aa, Artificial Sequence
  Synthetic
GRKKRRQRRRPPQ

SEQ ID NO: 32 - protein, 34 aa, Artificial Sequence
  Synthetic
DAATATRGRSAASRPTERPRAPARSASRPRRPVE

SEQ ID NO: 33 - protein, 27 aa, Artificial Sequence
  Synthetic
GWTLNSAGYLLGLINLKALAALAKKIL

SEQ ID NO: 34 - protein, 12 aa, Artificial Sequence
  Synthetic
PLSSIFSRIGDP

SEQ ID NO: 35 - protein, 16 aa, Artificial Sequence
  Synthetic
AAVALLPAVLLALLAP

SEQ ID NO: 36 - protein, 12 aa, Artificial Sequence
  Synthetic
AAVLLPVLLAAP

SEQ ID NO: 37 - protein, 15 aa, Artificial Sequence
  Synthetic
VTVLALGALAGVGVG

SEQ ID NO: 38 - protein, 21 aa, Artificial Sequence
  Synthetic
GALFLGWLGAAGSTMGAWSQP

SEQ ID NO: 39 - protein, 27 aa, Artificial Sequence
  Synthetic
GWTLNSAGYLLGLINLKALAALAKKIL

SEQ ID NO: 40 - protein, 18 aa, Artificial Sequence
  Synthetic
KLALKLALKALKAALKLA

SEQ ID NO: 41 - protein, 21 aa, Artificial Sequence
  Synthetic
KETWWETWWTEWSQPKKKRKV

SEQ ID NO: 42 - protein, 16 aa, Artificial Sequence
  Synthetic
KAFAKLAARLYRKAGC

SEQ ID NO: 43 - protein, 16 aa, Artificial Sequence
  Synthetic
KAFAKLAARLYRAAGC

SEQ ID NO: 44 - protein, 16 aa, Artificial Sequence
  Synthetic
AAFAKLAARLYRKAGC

SEQ ID NO: 45 - protein, 16 aa, Artificial Sequence
  Synthetic
KAFAALAARLYRKAGC

SEQ ID NO: 46 - protein, 16 aa, Artificial Sequence
  Synthetic
KAFAKLAAQLYRKAGC

SEQ ID NO: 47 - protein, 15 aa, Artificial Sequence
  Synthetic
GGGGYGRKKRRQRRR

SEQ ID NO: 48 - protein, 11 aa, Artificial Sequence
  Synthetic
YGRKKRRQRRR

SEQ ID NO: 49 - DNA, 489 bp, Pseudomonas aeruginosa
atggctgttgatatgttcatcaagatcggcgacgtcaagggtgagtccaaggacaagactcacgccgaggaaatcgacgtgctggcatggagctggggcatgtcccagtccgggtcgatgcacatgggcggtggcggcggcgccggcaaggtcaacgtgcaggacctgtcgttcaccaagtacatcgacaagtccacgcccaacctgatgatggcctgctccagcggcaagcactatccgcaggcgaagctgaccatccgcaaggccggcggcgagaaccaggtcgagtacctgatcatcaccctgaaggaagtcctggtgtcctcggtgagcaccggcggcagcggtggcgaggatcgcctgaccgagaacgtgaccctgaacttcgcccaggtccaggtcgactaccagccgcagaaggcggatggcgcgaaggacggcggtccggtcaagtacggctggaacatccgccagaacgtgcaggcctga

SEQ ID NO: 50 - protein, 162 aa, Pseudomonas aeruginosa
MAVDMFIKIGDVKGESKDKTHAEEIDVLAWSWGMSQSGSMHMGGGGGAGKVNVQDLSFTKYIDKSTPNLMMACSSGKHYPQAKLTIRKAGGENQVEYLIITLKEVLVSSVSTGGSGGEDRLTENVTLNFAQVQVDYQPQKADGAKDGGPVKYGWNIRQNVQA

SEQ ID NO: 51 - DNA, 1932 bp, Pseudomonas aeruginosa
atgcaactgacccgcctggtccaggtggattgcccgctggggccggacgtgctgctgttgcagcgcatggagggacgcgaggaactgggacggctgttcgcctacgagctgcacctggtatcggaaaatcccaacctgccgctggagcagttgctcggcaagccgatgagcctgtcgctggagctgcccggcggcagccggcgcttctttcacggcatcgtcgcgcgctgtagccaggtggccgggcacggccagttcgccggctaccaggccaccctgcggccctggccgtggctgctgacgcgcacctcggactgccgcatcttccagaaccagagcgtgccggagatcatcaagcaggtgttccgcaacctcggcttttccgatttcgaggatgccctcacgcgcccctaccgcgagtgggaatactgcgtgcagtaccgcgagaccagcttcgacttcatcagccggctgatggaacaggaaggcatctactactggttccgccatgagcagaagcgccacatcctggtgctctccgacgcctacggcgcgcatcgcagcccgggtggctacgccagcgtgccgtactacccgccgaccctcggccatcgcgagcgcgaccacttcttcgactggcagatggcacgcgaggtccagcccggttcgctgaccctcaacgactacgacttccagcgccccggcgcgcgcctggaggtgcgttcgaacatcgcccggccgcacgcggcggccgactacccgctgtacgactatcccggcgaatacgtgcagagccaggacggcgagcagtacgcgcgcaaccgcatcgaggcgatccaggcgcagcacgagcgcgtgcgcctgcgcggcgtggtgcgcgggatcggcgccgggcacctgttccgcctgagcggctatccgcgcgatgaccagaaccgcgagtacctggtggtcggcgccgaataccgggtggtccaggaactctacgaaaccggcagcggcggcgccggctcgcagttcgagagcgagctggactgcatcgacgccagccagtcgttccgtctcctgccgcagactccggtaccggtggtgcggggtccgcagaccgcggtggtggtcggacccaagggcgaggagatctggaccgaccagtacggccgggtcaaggtgcacttccactgggatcgccacgaccagtcgaacgagaacagctcctgctggattcgcgtgtcccaggcctgggccgggaagaactggggttcgatgcagatcccgcggatcggccaggaagtgatcgtcagcttcctcgaaggcgacccggaccggccgatcatcaccgggcgggtctacaacgccgagcagacggtgccctacgagctgccggcgaacgccacccagagcgggatgaagagccgttcgagcaagggcggcacgccggccaacttcaacgagatccgcatggaggacaagaagggcgccgagcagttatacatccacgccgagcgcaaccaggacaacctggtcgagaacgatgcctcgctgtcggtcggccacgaccgcaacaagagcatcggccacgacgagctggcgcgcatcggcaacaaccgcacccgcgcggtgaagctcaacgacaccctgttggtgggcggggcgaagagcgacagcgtcaccggcacctacctgatcgaggccggcgcgcagatccgcctggtctgcggcaagagcgtggtggagttcaacgccgacggcaccatcaatatctccggcagcgccttcaacctctacgccagcggcaacggcaacatcgacaccggcggccgcctcgacctcaattccggcggcgccagcgaggtcgacgccaagggcaagggcgtgcagggcaccatcgacggccaggtacaggcgatgtttccgccgccggcgaagggctga

SEQ ID NO: 52 - protein, 643 aa, Pseudomonas aeruginosa
MQLTRLVQVDCPLGPDVLLLQRMEGREELGRLFAYELHLVSENPNLPLEQLLGKPMSLSLELPGGSRRFFHGIVARCSQVAGHGQFAGYQATLRPWPWLLTRTSDCRIFQNQSVPEIIKQVFRNLGFSDFEDALTRPYREWEYCVQYRETSFDFISRLMEQEGIYYWFRHEQKRHILVLSDAYGAHRSPGGYASVPYYPPTLGHRERDHFFDWQMAREVQPGSLTLNDYDFQRPGARLEVRSNIARPHAAADYPLYDYPGEYVQSQDGEQYARNRIEAIQAQHERVRLRGVVRGIGAGHLFRLSGYPRDDQNREYLVVGAEYRVVQELYETGSGGAGSQFESELDCIDASQSFRLLPQTPVPVVRGPQTAVVVGPKGEEIWTDQYGRVKVHFHWDRHDQSNENSSCWIRVSQAWAGKNWGSMQIPRIGQEVIVSFLEGDPDRPIITGRVYNAEQTVPYELPANATQSGMKSRSSKGGTPANFNEIRMEDKKGAEQLYIHAERNQDNLVENDASLSVGHDRNKSIGHDELARIGNNRTRAVKLNDTLLVGGAKSDSVTGTYLIEAGAQIRLVCGKSVVEFNADGTINISGSAFNLYASGNGNIDTGGRLDLNSGGASEVDAKGKGVQGTIDGQVQAMFPPPAKG

SEQ ID NO: 53 - DNA, 24 bp, Artificial Sequence
  Synthetic
ttcagcatgcttgcggctcgagtt

SEQ ID NO: 54 - protein, 172 aa, Artificial Sequence
  Synthetic
MKLLAGSFAALFLSLSAQAADCTFTQLEIVPQFGSPNMFGGEDEHVRVMFSNEDPNDDNPDAFPEPPVYLADRDSGNDCRIEDGGIWSRGGVFLSQDGRRVLMHEFSGSSAELVSYDSATCKVVHREDISGQRWAVDKDGLRLGQKCSGESVDSCAKIVKRSLAPFCQTAKK

SEQ ID NO: 55 - protein, 77 aa, Artificial Sequence
  Synthetic
MNLKPQTLMVAIQCVAARTRELDAQLQNDDPQNAAELEQLLVGYDLAADDLKNAYEQALGQYSGLPPYDRLIEEPAS

SEQ ID NO: 56 - protein, 145 aa, Artificial Sequence
  Synthetic
MKTVALILASLALLACTAESGVDFDKTLTHPNGLVVERPVGFDARRSAEGFRFDEGGKLRNPRQLEVQRQDAPPPPDLASRRLGDGEARYKVEEDDGGSAGSEYRLWAAKPAGARWIVVSASEQSEDGEPTFALAWALLERARLQ

SEQ ID NO: 57 - protein, 15 aa, Artificial Sequence
  Synthetic
YDGDVGRYLHPDKEC